(19) United States
(12) Patent Application Publication
(10) Pub. No.: US 2020/0300861 A1
(43) Pub. Date: Sep. 24, 2020

(54) Isolation of Single Cells and Uses Thereof

(71) Applicant: Augmenta Bioworks, Inc., Menlo Park, CA (US)

(72) Inventors: Marco Antonio Mena, Santa Clara, CA (US); Christopher J. Emig, Menlo Park, CA (US); Kim-Xuan Nguyen, Sunnyvale, CA (US)

(73) Assignee: Augmenta Bioworks, Inc., Menlo Park, CA (US)

(21) Appl. No.: 16/826,189

(22) Filed: Mar. 21, 2020

Related U.S. Application Data
(60) Provisional application No. 62/822,500, filed on Mar. 22, 2019.

(51) Int. Cl. G01N 33/68; G01N 33/543; C12Q 1/6869; G01N 33/58

(52) U.S. Cl. CPC G01N 33/6818; G01N 33/582; C12Q 1/6869; G01N 33/543

(57) ABSTRACT

The present invention relates generally to the field of immune binding proteins and method for obtaining immune binding proteins from genomic or other sources. The present invention also relates to methods and apparati for obtaining single cells that express immune binding proteins. The single cells expressing the immune binding proteins can be obtained from a patient that has had an effective immune response to a disease state (e.g., cancer or an infectious agent). The methods and apparati of the disclosure can be used to obtain immune cells that produce immune binding proteins responsible for the effective immune response. The methods and apparati of the disclosure can also be used to obtain cells that express a polypeptide (e.g., a receptor, a secreted protein, a cytokine, or a recombinant protein) or other factor of interest.

This application claims priority to U.S. provisional application Ser. No. 62/822,500 filed Mar. 22, 2019.

### REFERENCE TO SEQUENCE LISTING, TABLE OR COMPUTER PROGRAM

The official copy of the Sequence Listing is submitted concurrently with the specification as an ASCII formatted text file via EFS-Web, with a file name of “ABW014_ST25.txt”, a creation date of Mar. 19, 2020, and a size of 11 kilobytes. The Sequence Listing filed via EFS-Web is part of the specification and is incorporated in its entirety by reference herein.

### BACKGROUND OF THE DISCLOSURE

There is considerable interest in being able to discover antibodies to specific antigens. Such antibodies are useful as research tools and for diagnostic and therapeutic applications. However, the identification of such useful antibodies is difficult and once identified, these antibodies often require considerable redesign before they are suitable for therapeutic applications in humans.

Many methods for identifying antibodies involve display of antibody libraries derived by amplification of nucleic acids from B cells or other tissues. These approaches have limitations that limit the useful antibodies obtained from the library. For example, most antibody libraries do not pair the heavy and light chains obtained from memory B-cells or plasma cells that have mounted an effective immune response against an immunological challenge. In addition, most human antibody libraries known contain only the antibody sequence diversity that can be experimentally captured or cloned from a biological source (e.g., B cells). Accordingly, such libraries may over-represent some sequences, while completely lacking or under-representing other sequences especially paired light and heavy chains that form useful antibodies, particularly those from a successful immune response.

It is an object of this invention to provide libraries of immune binding proteins that are enriched for useful immune binding proteins. It is also an object of the invention to provide methods for making such libraries that are enriched for useful multimers of immune binding proteins. It is a further object of the invention to provide methods for amplifying nucleic acids from B-cells and plasma cells so that the pairing of light and heavy chains is maintained. It is an object of the invention to obtain libraries of antibodies relevant to disease therapies by obtaining paired light and heavy chain antibodies from individuals whom have mounted antibody responses against a variety of immunologic challenges related to, for example, infectious diseases (an infectious agent), cancer, auto-immune disease, neurodegenerative disease, and allergies.

### SUMMARY OF THE INVENTION

The disclosure relates to nucleic acids encoding immune binding proteins that preserve the in vivo multimeric associations of the immune polypeptide chains making up the immune binding protein (e.g., antibodies, T-lymphocyte receptors, or innate immunity receptors). Immune binding protein libraries enriched for nucleic acids encoding multimers that functionally represent the multimeric complexes found in the cells from which the immune binding protein library can be obtained. The nucleic acids encoding the polypeptide chains for immune binding proteins can be derived from individuals whom have mounted an immune response relevant to, for example, an infectious disease, a cancer, an autoimmune disease, an allergy, or a neurodegenerative disease. The infectious disease can be caused by an influenza virus. The infectious disease can be caused by an infectious agent such as a virus, for example, HIV, Ebola, Zika, HSV, RSV or CMV, or a pathogenic bacteria. The cancer can be a melanoma. The cancer can be one that responds to immunotherapy.

The disclosure also relates to nucleic acids encoding polypeptide chains for immune binding proteins (e.g., light and heavy chain antibody polypeptides) that preserve the in vivo functional pairing of the polypeptide chains (e.g., light and heavy chains of an antibody). The immune binding protein libraries can be enriched for functional multimers of nucleic acids encoding the polypeptide chains that make up the immune binding protein (e.g., light and heavy chains of an antibody) and which were associated together in the repertoire from which the immune binding protein library was obtained. The nucleic acids encoding associated polypeptide chains for the immune binding protein (e.g., paired light and heavy chains) can be derived from individuals whom have mounted an immune response relevant to, for example, an infectious disease, a cancer, an autoimmune disease, an allergy, or a neurodegenerative disease.

The disclosure also relates to a plurality of nucleic acids comprising a plurality of polynucleotides encoding a first chain of a multimeric immune binding protein, a plurality of polynucleotides encoding a second chain of a multimeric immune binding protein, wherein each polynucleotide encoding the first chain of the multimeric immune binding protein is paired with the polynucleotide encoding the second chain of the immune binding protein to form a plurality of pairs of polynucleotides encoding the first chain and the second chain, wherein the plurality of pairs of polynucleotides represent a plurality of pairs of first chains and second chains as they are found in a plurality of host cells from which the multimeric immune binding proteins are derived. The multimeric immune binding protein can be an antibody, a T-cell receptor or an innate immunity receptor. In some embodiments, the antibody is a scFv, a Fab, a F(ab′)₂, a Fab′, a Fv, or a diabody. In some embodiments, the antibody is an IgG, an IgM, an IgA, an IgD, or an IgE. The antibody can be from a B-cell, a plasma cell, a B memory cell, a pre-B-cell or a progenitor B-cell. The T-cell receptor can be a single chain T-cell receptor. The T-cell receptor can be from a CD8+ T-cell, a CD4+ T-cell, a regulatory T-cell, a memory T-cell, a helper T-cell, or a cytotoxic T-cell. The multimeric immune binding protein can be from a natural killer cell, a macrophage, a monocyte, or a dendritic cell.

Individual cells containing nucleic acids encoding the immune binding proteins can be placed into microwells and/or an emulsion. Primers for the forward (F) and reverse (R) directions of the nucleic acids encoding the polypeptides for the immune binding protein (e.g., antibody heavy (H) and light (L) chains) can be introduced (e.g., HF, HR, LF, and LR), as well as a polymerase enzyme and dNTPs to carry out template-directed amplification. The F1 (e.g., HF) and R2 (e.g., LR) primers (or alternatively the LF and HR primers) can contain an overlap extension region (OE) such that during cycled amplification these primers mutually extend each other. A joint polypeptide (such as a scFv) can be encoded by the amplified nucleic acids, the OE region can also encode an amino acid linker sequence (FIG. 1A). The amplified nucleic acids can be used in a sequencing reaction and one or more of the primers can include a barcode region (e.g., BC1, BC2, BC3 and/or BC4) (FIG. 1B). The amplification reaction can be carried out, resulting in a nucleic acid which codes for the two polypeptide chains of the immune binding protein (e.g., both a heavy and a light chain of an antibody). The nucleic acid obtained from each well and/or emulsion can be homogeneous and can encode the antibody made by the single cell placed in the microwell and/or emulsion. Nucleic acids obtained from the wells and/or emulsions can be pooled to form a library of immune binding proteins (e.g., heavy/light chain pairs) that reflect the association of polypeptides (e.g., pairing of the antibody chains) from the source cells or genetic material.

The resulting pool of nucleic acids encoding associated polypeptides of the immune binding protein (e.g., paired heavy and light chains for and antibody) can be cloned into an expression vector or can be processed for sequencing. The expression vector can be engineered for phage display, yeast display, or other display technology. The expression vector can be for secretion expression and recombinant production of the immune binding protein. The expression vector can be for making a library of chimeric antigen receptors, where each CAR has one of the associated immune binding protein clones obtained from the amplification reaction. Primers corresponding to heavy chains or light chains may be targeted to single isotypes of antibodies (e.g., IgG), or pools of primers corresponding to all available isotypes or some fraction thereof may be used.

Primers for the polypeptide chains of the immune binding protein (e.g., light chain and heavy chains of an antibody) can be linked together so that each primer can be capable of priming a reaction. A 5′ azide-alkyne reaction (“Click”) coupling can bring together the primers. A dual primer can be incubated with single cells in a well or emulsion, and nucleic acids can be obtained where a nucleic acid encoding one polypeptide chain of the immune binding protein is linked to a nucleic acid encoding the associated polypeptide chain of the same immune binding protein (e.g., a heavy chain is linked to a nucleic acid encoding the paired light chain). A microsurface (e.g., bead or microwell) can be prepared and can contain primer sequences that are capable of binding nucleic acids encoding multiple, associated polypeptides of the immune binding protein (e.g., heavy and light chain nucleic acids). Following mRNA capture, cDNA synthesis or PCR from a single cell in a spatial confinement with the primers in the well or on the bead, nucleic acids encoding the associated polypeptide chains (e.g., paired heavy and light chains) become co-located with the primers of the solid phase.

Nucleic acid probes for nucleic acids encoding associated polypeptides of the immune binding protein (e.g., heavy and light chain polypeptides) can be placed on a solid surface. These probes for nucleic acids encoding associated polypeptides of the immune binding protein (e.g., heavy and light chain polypeptides) can be interrogated with nucleic acids, e.g., mRNA, from a single cell. The probes on the solid phase will capture nucleic acids encoding the associated polypeptides of the immune binding protein (e.g., heavy and light chain polypeptides) from the cell. Captured mRNA can be reverse transcribed to make paired cDNAs encoding associated polypeptides of the immune binding protein (e.g., heavy and light chain polypeptides) from a single cell.

The nucleic acids encoding the subunits of the immune binding protein can be bar coded to enable identification of unique molecules. A solid phase with a cell-specific barcode can be made with spatially confined PCR reactions of a plurality of single template molecules containing a linker/adapter primer sequence, a random barcode sequence, and a secondary primer sequence. A limited dilution of template molecules can be used, and the template molecule can be linked to a solid phase at very low loading rates to ensure only a single molecule is available as a template at each site. At least one of these primers in this PCR reaction can be attached to the solid phase. Additional molecules may be added to load additional sites, knowing that previously bound sites are incapable of reacting because they were exhausted during previous rounds of PCR. Oligonucleotides can be attached at an extremely low loading rate to a surface and beads can be flowed over the surface to ensure that each bead binds a single oligonucleotide. Beads can be reflowed over the surface without being subjected to the constraints of poissonian loading. Each bound bead can be guaranteed to have one and only one template sequence. Each spatially confined site (either a position or well on a patterned surface, or bead in emulsion) can contain the same barcoded DNA in close proximity, whereas other sites can each contain separate barcoded DNA in close proximity originating from other single molecule templates. Single stranded DNA can be generated through the use of a 5′ nuclease or denaturation of the uncoupled second strand. The secondary primer sequence can be available to perform a subsequent barcode extension reaction or can be used directly to capture nucleic acids from single cells. The bead can be ligated to a sequence containing a linker section and a fully random sequence to serve as a unique molecular identifier, and a tertiary primer sequence. The tertiary primer sequence can be available to perform a subsequent barcode extension reaction or can be used directly to capture nucleic acids from single cells.

Antigens can be identified for the immune binding proteins described herein. Nucleic acids can encode the subunits (or pairs) of an immune binding protein and the antigen bound by the immune binding protein. A three-way coupling can be made between nucleic acids encoding associated polypeptides of the immune binding protein (e.g., heavy and light chain polypeptides), and an antigen that is barcoded with an antigen-specific sequence. Antibodies can be displayed on the surface of a cell, probed with a population of barcoded antigens, and then the resulting conjugates can be encapsulated into a microwell or an emulsion, and sequence amplification methods can be utilized to recover the sequence of the associated polypeptides of the immune binding protein (e.g., heavy and light chain polypeptides) and the barcoded antigen sequence. A plurality of antigens can be bar coded. The bar-coded antigens can subsequently be screened against immune binding proteins to find the immune binding proteins that bind to specific antigens. This screening can be done with immune binding proteins from the libraries described herein, immune cells obtained from a subject who is naïve to the antigen, or immune cells obtained from a subject who has mounted a relevant immune response (e.g., an immune response relevant to an infectious disease, a cancer, an autoimmune disease, an allergy, or a neurodegenerative disease). The immune cells paired with bar coded antigens can then be used in the amplification methods to obtain nucleic acids encoding immune binding proteins and the immune binding proteins.

Nucleic acids encoding the immune binding proteins can be sequenced. The sequencing can be done by high-throughput sequencing. The sequence information obtained can be used for putative lineage information based on sequence alignment.

A method can be provided for generating a population of cell containing gel-beads, wherein the cells can be encapsulated in a water/oil emulsion to create a plurality of droplets. Once formed, the droplets are subsequently exposed to a gelation reagent or a combination of gelation reagents to yield a population of gel-beads. Gelation can be achieved by methods suitable for the gelation agents such as, for example, rapid cooling (e.g., for agarose), treatment with light (for light polymerizable monomers), treatment with temperature or treatment by means of an ion or free radical. Subsequently, the gel-beads can be collected, captured, or attached to a suitable surface (e.g., a chip), and the collected, captured, or attached gel-beads can be treated by a variety of techniques to assay or treat the contents of the gel-bead.

The gelation reagent can be an alginate, agarose, acrylamide or a polyalkylene glycol, such as PEG. The gelation reagent can also be combined with a cross-linking agent and can also include, for example, a temperature sensitive polymer, light sensitive polymer, a specific ion-sensitive polymer or a dual-or-multi-sensitive polymer.

Droplets formed through encapsulation of a cell in a water/oil emulsion, can be stabilized through employing a stabilization membrane prior to exposure of the droplets to the gelation reagent.

The gelation reagent can be agarose and can be present in an amount of about 0.5% to about 5.0% in the formation of a population of gel-beads. The gelation reagent can be an alginate and can be present in an amount of about 0.5% to about 5.00/% in the formation of a population of gel-beads.

The gelation reagent can be acrylamide and can be present in an amount of 3% to about 20% monomer and further comprises up to about 50/% of a crosslinker in the formation of a population of gel-beads.

The gelation reagent can contain a PEG-dendrimer functionalized with a reactive moiety, such as Dibenzocyclooctyne (“DBCO”), N-hydroxysuccinirnidyl (“NHS”), acrylate, azide, amine or thiol and a multifunctionalized PEG with a reactivity toward the functionalized dendrimer, such as azide, amine, thiol, DBCO, NHS, or acrylate, respectively.

The gelation solution may contain inclusions of unfunctionalized polymer to create void spaces in the polymer matrix.

The polymer used to make these inclusions can be chemically, enzymatically or photolytically cleavable, such as a dithiol containing polymer with DTT (chemically), an agarose polymer cleavable with agarose (enzymatically), a polypeptide cleavable with a protease (enzymatically), an alginate cleavable with EDTA (chemically), a desthiobiotin functionalized dendrimer crosslinked to streptavidin cleavable with introduction of biotin (chemically), or a polymer containing o-nitrobenzyl groups in the backbone (photocleavable).

Methods are also provided for generating a population of cell containing core-shell beads, wherein the cells can be encapsulated in a water/oil emulsion to provide a plurality of droplets. These droplets can be characterized by having an inner portion and an outer portion. When these droplets are exposed to a gelation reagent or a combination of gelation reagents and selected polymers, a unique population of core-shell beads can be formed wherein the inner portion is comprised of a liquid core and the outer portion is comprised of a gelation material. Subsequently, the formed core-shell beads can be attached to a suitable microsurface, such as a chip, and treated by a variety of techniques. These techniques include those described above including, for example, rapid cooling, treatment with light, treatment with temperature or treatment by the introduction of an ion. The population of core-shell beads may contain a scaffolding and can also include a capture agent.

A high-throughput system and/or “HTS” device for single-cell isolation is also described herein. The device can include, for example, an inverted microscope and camera component, a substrate component, a cell picker component a robotic arm component wherein the device is capable of isolating individual cells from a heterogeneous population of cells. The HTS device is capable of identifying and selecting single cells and can also dispose single cells into an array, wells, on a substrate, etc. These single cells can be screened for secreted products, which include, for example, antibodies, cytokines and/or other metabolites.

Methods for selecting individual cells from a population of cells can utilize the HTS device that includes, for example, an inverted microscope and camera component, a substrate component, a cell picker component and a robotic arm component and subsequently introducing a sample containing one or more cells into the substrate component, and selecting an individual cell from the sample.

Methods described herein also can use arrays of single cells made by the HTS device. Such arrays can be made on a substrate, a microwell plate, or other container. Methods using arrays of cells can identify cells making an antibody which binds to an antigen(s) of interest. Methods using arrays of cells can identify cells making a receptor (e.g., a T-cell receptor) or ligand that bind to an antigen(s) of interest. Methods using arrays of cells can also identify cells that are making a protein of interest. The protein of interest can be a recombinant protein, and enzyme, an immune binding protein, a cytotoxic protein, etc. Methods using the array of cells can identify cells that are producing large amounts of a protein of interest for making an expression cell line.

### BRIEF DESCRIPTION OF THE FIGURES

FIG. 1 shows components of a single cell and/or single particle selecting device.

FIG. 2 shows an example of a HTS device for picking single cells and/or single particles.

FIG. 3 shows a cell/particle picker with a glass capillary and stage components.

FIG. 4 shows plate mount, a stage mount and a cell/particle picker mount.

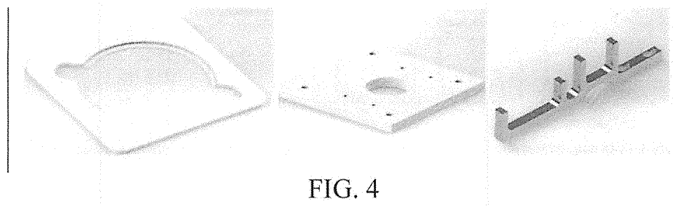

FIG. 5 shows a data acquisition and analysis of mock cells in a nanowell array.

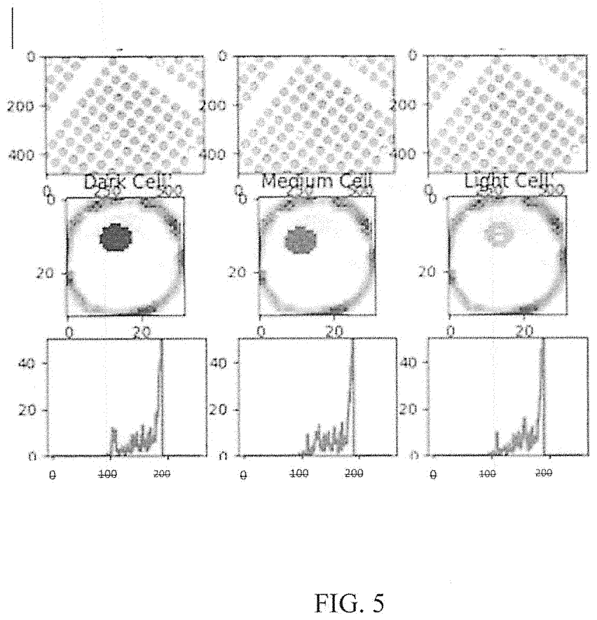

FIG. 6 shows a work flow chart for obtaining clones expressing a desired antigen binding protein using a single cell selecting device.

FIG. 7 shows components of an alternative single cell and/or single particle selecting device.

FIG. 8 shows the components for another single cell and/or single particle selecting device.

### DETAILED DESCRIPTION OF THE INVENTION

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

It must be noted that as used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements or use of a “negative” limitation. Numerical limitations given with respect to concentrations or levels of a substance are intended to be approximate, unless the context clearly dictates otherwise. Thus, where a concentration is indicated to be (for example) 10 micrograms (“μg”), it is intended that the concentration be understood to be at least approximately or about 10 μg.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

### Definitions

As used herein, an “antibody” refers to a protein functionally defined as a binding protein and structurally defined as comprising an amino acid sequence that is recognized as being derived from the framework region of an immunoglobulin encoding gene of an animal producing antibodies. An antibody can consist of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist as intact immunoglobulins or as a number of well-characterized fragments. Thus, for example, pepsin digests an antibody below the disulfide linkages in the hinge region to produce F(ab)′₂, a dimer of Fab which itself is a light chain joined to VH-CH1 by a disulfide bond. The F(ab)′₂ may be reduced under mild conditions to break the disulfide linkage in the hinge region thereby converting the (Fab′)₂ dimer into an Fab′ monomer. The Fab′ monomer is essentially an Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that fragments can be synthesized de novo either chemically or by utilizing recombinant DNA methodology. Thus, the term antibody, as used herein also includes antibody fragments either produced by the modification of whole antibodies or synthesized using recombinant DNA methodologies. Preferred antibodies include V_{H}-V_{L} dimers, including single chain antibodies (antibodies that exist as a single polypeptide chain), such as single chain Fv antibodies (sFv or scFv) in which a variable heavy and a variable light region are joined together (directly or through a peptide linker) to form a continuous polypeptide. The single chain Fv antibody is a covalently linked V_{H}-V_{L} heterodimer which may be expressed from a nucleic acid including V_{H}- and V_{L}-encoding sequences either joined directly or joined by a peptide-encoding linker (e.g., Huston, et al. Proc. Nat. Acad. Sci. USA, 85:5879-5883, 1988). While the V_{H} and V_{L} are connected to each as a single polypeptide chain, the V_{H} and V_{L} domains associate non-covalently. Alternatively, the antibody can be another fragment. Other fragments can also be generated, including using recombinant techniques. For example, Fab molecules can be displayed on phage if one of the chains (heavy or light) is fused to g3 capsid protein and the complementary chain exported to the periplasm as a soluble molecule. The two chains can be encoded on the same or on different replicons; the two antibody chains in each Fab molecule assemble post-translationally and the dimer is incorporated into the phage particle via linkage of one of the chains to g3p (see, e.g., U.S. Pat. No. 5,733,743). The scFv antibodies and a number of other structures converting the naturally aggregated, but chemically separated light and heavy polypeptide chains from an antibody V region into a molecule that folds into a three-dimensional structure substantially similar to the structure of an antigen-binding site are known to those of skill in the art (see e.g., U.S. Pat. Nos. 5,091,513, 5,132,405, and 4,956,778). In some embodiments, the scFv is a diabody as described in Holliger et al., Proc. Nat'l Acad. Sci. vol. 90, pp. 6444-6448 (1993), which is incorporated by reference in its entirety for all purposes. In some embodiments, antibodies include all those that have been displayed on phage or generated by recombinant technology using vectors where the chains are secreted as soluble proteins, e.g., scFv, Fv, Fab, pr (Fab′)₂ or generated by recombinant technology using vectors where the chains are secreted as soluble proteins. Antibodies can also include diantibodies and miniantibodies.

Antibodies of the invention also include heavy chain dimers, such as antibodies from camelids. Since the V_{H} region of a heavy chain dimer IgG in a camelid does not have to make hydrophobic interactions with a light chain, the region in the heavy chain that normally contacts a light chain is changed to hydrophilic amino acid residues in a camelid. V_{H} domains of heavy-chain dimer IgGs are called V_{HH} domains.

In camelids, the diversity of antibody repertoire is determined by the complementary determining regions (CDR) 1, 2, and 3 in the V_{H} or V_{HH} regions. The CDR3 in the camel V_{HH} region is characterized by its relatively long length averaging 16 amino acids (Muyldermans et al., 1994, Protein Engineering 7(9): 1129). This is in contrast to CDR3 regions of antibodies of many other species. For example, the CDR3 of mouse V_{H} has an average of 9 amino acids.

Libraries of camelid-derived antibody variable regions, which maintain the in vivo diversity of the variable regions of a camelid, can be made by, for example, the methods disclosed in U.S. Patent Application Ser. No. 20050037421, published Feb. 17, 2005.

As used herein, “HA,” “NB,” and “NA” respectively mean hemagglutinin, NB protein and neuraminidase. HA, NB and NA are antigenic glycoproteins located on the surface of influenza viruses. These glycoproteins are responsible for the binding the virus to the cell that is to be infected and processes that result in infection with the virus.

As used herein, the term “naturally occurring” means that the components are encoded by a single gene that was not altered by recombinant means and that pre-exists in an organism, e.g., in an antibody library that was created from naive cells or cells that were exposed to an antigen.

As used herein, the term “antigen” refers to substances that are capable, under appropriate conditions, of inducing a specific immune response and of reacting with the products of that response, such as, with specific antibodies or specifically sensitized T-lymphocytes, or both. Antigens may be soluble substances, such as toxins and foreign proteins, or particulates, such as bacteria and tissue cells; however, only the portion of the protein or polysaccharide molecule known as the antigenic determinant (epitopes) combines with the antibody or a specific receptor on a lymphocyte. More broadly, the term “antigen” may be used to refer to any substance to which an antibody binds, or for which antibodies are desired, regardless of whether the substance is immunogenic. For such antigens, antibodies may be identified by recombinant methods, independently of any immune response.

As used herein, the term “epitope” refers to the site on an antigen or hapten to which specific B cells and/or T cells respond. The term is also used interchangeably with “antigenic determinant” or “antigenic determinant site”. Epitopes include that portion of an antigen or other macromolecule capable of forming a binding interaction that interacts with the variable region binding pocket of an antibody.

As used herein, the term “binding specificity” of an antibody refers to the identity of the antigen to which the antibody binds, preferably to the identity of the epitope to which the antibody binds.

As used herein, the term “chimeric polynucleotide” means that the polynucleotide comprises regions which are wild-type and regions which are mutated. It may also mean that the polynucleotide comprises wild-type regions from one polynucleotide and wild-type regions from another related polynucleotide.

As used herein, the term “complementarity-determining region” or “CDR” refer to the art-recognized term as exemplified by the Kabat and Chothia. CDRs are also generally known as hypervariable regions or hypervariable loops (Chothia and Lesk (1987) J Mol. Biol. 196: 901; Chothia et al. (1989) Nature 342: 877; E. A. Kabat et al., Sequences of Proteins of Immunological Interest (National Institutes of Health, Bethesda, Md.) (1987); and Tramontano et al. (1990) J Mol. Biol. 215: 175). “Framework region” or “FR” refers to the region of the V domain that flank the CDRs. The positions of the CDRs and framework regions can be determined using various well known definitions in the art, e.g., Kabat, Chothia, international ImMunoGeneTics database (IMGT), and AbM (see, e.g., Johnson et al., supra; Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human V_{H} segments J. Mol. Biol. 227, 799-817; Al-Lazikani et al., J. Mol. Biol 1997, 273(4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1):207-9 (2001); MacCallum et al, Antibody-antigen interactions: Contact analysis and binding site topography, J. Mol. Biol., 262 (5), 732-745 (1996); and Martin et al, Proc. Natl Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 1996).

As used herein, the term “hapten” is a small molecule that, when attached to a larger carrier such as a protein, can elicit an immune response in an organism, e.g., such as the production of antibodies that bind specifically to it (in either the free or combined state). A “hapten” is able to bind to a preformed antibody, but may fail to stimulate antibody generation on its own. In the context of this invention, the term “hapten” includes modified amino acids, either naturally occurring or non-naturally occurring. Thus, for example, the term “hapten” includes naturally occurring modified amino acids such as phosphotyrosine, phosphothreonine, phosphoserine, or sulphated residues such as sulphated tyrosine (sulphotyrosine), sulphated serine (sulphoserine), or sulphated threonine (sulphothreonine); and also include non-naturally occurring modified amino acids such as p-nitro-phenylalanine.

As used herein, the term “heterologous” when used with reference to portions of a polynucleotide indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid. Similarly, a “heterologous” polypeptide or protein refers to two or more subsequences that are not found in the same relationship to each other in nature.

As used herein, the term “host cell” refers to a prokaryotic or eukaryotic cell into which the vectors of the invention may be introduced, expressed and/or propagated. A microbial host cell is a cell of a prokaryotic or eukaryotic micro-organism, including bacteria, yeasts, microscopic fungi and microscopic phases in the life-cycle of fungi and slime molds. Typical prokaryotic host cells include various strains of E. coli. Typical eukaryotic host cells are yeast or filamentous fungi, or mammalian cells, such as Chinese hamster ovary cells, murine NIH 3T3 fibroblasts, human embryonic kidney 193 cells, or rodent myeloma or hybridoma cells.

As used herein, the term “immunological response” to a composition or vaccine is the development in the host of a cellular and/or antibody-mediated immune response to a composition or vaccine of interest. Usually, an “immunological response” includes but is not limited to one or more of the following effects: the production of antibodies, B cells, helper T cells, and/or cytotoxic T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction or lack of symptoms normally displayed by an infected host, a quicker recovery time and/or a lowered viral titer in the infected host.

As used herein, the term “isolated” refers to a nucleic acid or polypeptide separated not only from other nucleic acids or polypeptides that are present in the natural source of the nucleic acid or polypeptide, but also from polypeptides, and preferably refers to a nucleic acid or polypeptide found in the presence of (if anything) only a solvent, buffer, ion, or other component normally present in a solution of the same. The terms “isolated” and “purified” do not encompass nucleic acids or polypeptides present in their natural source.

As used herein, Fluorescence activated cell sorting (“FACS”) of live cells separates a population of cells into sub-populations based on fluorescent labeling. Sorting involves more complex mechanisms in the flow cytometer than a non-sorting analysis. Cells stained using fluorophore-conj ugated antibodies are separated from one another depending on the fluorophore with which they have been stained and/or the intensity of staining. For example, a cell expressing one cell marker may be detected using an FITC-conjugated antibody that recognizes the marker, and another cell type expressing a different marker could be detected using a PE-conjugated antibody specific for that marker.

As used herein, the term “mammal” refers to warm-blooded vertebrate animals all of which possess hair and suckle their young.

As used herein, “percentage of sequence identity” and “percentage homology” are used interchangeably herein to refer to comparisons among polynucleotides or polypeptides, and are determined by comparing two optimally aligned sequences over a comparison window, where the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, Adv Appl Math. 2:482, 1981; by the homology alignment algorithm of Needleman and Wunsch, J Mol Biol. 48:443, 1970; by the search for similarity method of Pearson and Lipman, Proc Natl Acad Sci. USA 85:2444, 1988; by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Greene Publishing Associates, Inc. and John Wiley & Sons, Inc., (1995 Supplement). Examples of algorithms that are suitable for determining percent sequence identity and sequence similarity are the BLAST and BLAST 2.0 algorithms, which are described in Altschul et al., J. Mol. Biol. 215:403-410, 1990; and Altschul et al., Nucleic Acids Res. 25(17):3389-3402, 1977; respectively. Software for performing BLAST analyses is publicly available through the National Center for Biotechnology Information website. BLAST for nucleotide sequences can use the BLASTN program with default parameters, e.g., a wordlength (W) of 11, an expectation (E) of 10, M=5, N=−4, and a comparison of both strands. BLAST for amino acid sequences can use the BLASTP program with default parameters, e.g., a wordlength (W) of 3, an expectation (E) of 10, and the BLOSUM62 scoring matrix (see Henikoff and Henikoff, Proc Natl Acad Sci. USA 89:10915, 1989). Exemplary determination of sequence alignment and % sequence identity can also employ the BESTFIT or GAP programs in the GCG Wisconsin Software package (Accelrys, Madison Wis.), using default parameters provided.

As used herein, the terms “protein”, “peptide”, “polypeptide” and “polypeptide fragment” are used interchangeably herein to refer to polymers of amino acid residues of any length. The polymer can be linear or branched, it may comprise modified amino acids or amino acid analogs, and it may be interrupted by chemical moieties other than amino acids. The terms also encompass an amino acid polymer that has been modified naturally or by intervention; for example disulfide bond formation, glycosylation, lipidation, acetylation, phosphorylation, or any other manipulation or modification, such as conjugation with a labeling or bioactive component.

As used herein, the term “purified” means that the indicated nucleic acid or polypeptide is present in the substantial absence of other biological macromolecules, e.g., polynucleotides, proteins, and the like. In one embodiment, the polynucleotide or polypeptide is purified such that it constitutes at least 95% by weight, more preferably at least 99.8% by weight, of the indicated biological macromolecules present (but water, buffers, and other small molecules, especially molecules having a molecular weight of less than 1000 daltons, can be present)

As used herein, the term “recombinant nucleic acid” refers to a nucleic acid in a form not normally found in nature. That is, a recombinant nucleic acid is flanked by a nucleotide sequence not naturally flanking the nucleic acid or has a sequence not normally found in nature. Recombinant nucleic acids can be originally formed in vitro by the manipulation of nucleic acid by restriction endonucleases, or alternatively using such techniques as polymerase chain reaction. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention.

As used herein, the term “recombinant polypeptide” refers to a polypeptide expressed from a recombinant nucleic acid, or a polypeptide that is chemically synthesized in vitro.

As used herein, the term “recombinant variant” refers to any polypeptide differing from naturally occurring polypeptides by amino acid insertions, deletions, and substitutions, created using recombinant DNA techniques. Guidance in determining which amino acid residues may be replaced, added, or deleted without abolishing activities of interest, such as enzymatic or binding activities, may be found by comparing the sequence of the particular polypeptide with that of homologous peptides and minimizing the number of amino acid sequence changes made in regions of high homology.

Preferably, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

As used herein, the terms “repertoire” or “ ”library” refers to a library of genes encoding antibodies or antibody fragments such as Fab, scFv, Fd, LC, V_{H}, or V_{L}, or a subfragment of a variable region, e.g., an exchange cassette, that is obtained from a natural ensemble, or “repertoire”, of antibody genes present, e.g., in human donors, and obtained primarily from the cells of peripheral blood and spleen. In some embodiments, the human donors are “non-immune”, i.e., not presenting with symptoms of infection. In the current invention, a library or repertoire often comprises members that are exchange cassette of a given portion of a V region.

As used herein, the term “synthetic antibody library” refers to a library of genes encoding one or more antibodies or antibody fragments such as Fab, scFv, Fd, LC, V_{H}, or V_{L}, or a subfragment of a variable region, e.g., an exchange cassette, in which one or more of the complementarity-determining regions (CDR) has been partially or fully altered, e.g., by oligonucleotide-directed mutagenesis. “Randomized” means that part or all of the sequence encoding the CDR has been replaced by sequence randomly encoding all twenty amino acids or some subset of the amino acids.

As used herein, a T-cell” is defined to be a hematopoietic cell that normally develops in the thymus. T-cells include, but are not limited to, natural killer T cells, regulatory T cells, helper T cells, cytotoxic T cells, memory T cells, gamma delta T cells and mucosal invariant T cells. T-cells also include but are not limited to CD8+ T-cells, CD4+ T-cells, Th1 T-cells, and Th2 T-cells.

The singular terms “a”, “an”, and “the” include plural referents unless context clearly indicates otherwise. Similarly, the word “or” is intended to include “and” unless the context clearly indicates otherwise. Numerical limitations given with respect to concentrations or levels of a substance, such as an antigen, are intended to be approximate. Thus, where a concentration is indicated to be at least (for example) 200 μg, it is intended that the concentration be understood to be at least approximately “about” or “about” 200 μg.

### Immune Binding Proteins

In some embodiments, the immune binding protein is an antibody, a T-cell receptor, or an innate immunity receptor. In some embodiments, the immune binding protein is from a cell of the immune system including, for example, a B-cell, a plasma cell, a T-cell, a natural killer cell, a dendritic cell, or a macrophage.

In some embodiments, antibodies are immune binding proteins that are structurally defined as comprising an amino acid sequence recognized as being derived from the framework region of an immunoglobulin. In some embodiments, an antibody consists of one or more polypeptides substantially encoded by immunoglobulin genes or fragments of immunoglobulin genes. In some embodiments, the immunoglobulin genes include, for example, the kappa, lambda, alpha, gamma, delta, epsilon and mu constant region genes, as well as myriad immunoglobulin variable region genes. In some embodiments, antibody light chains are classified as either kappa or lambda. In some embodiments, antibody heavy chains are classified as gamma, mu, alpha, delta, or epsilon, which in turn define the immunoglobulin classes, IgG, IgM, IgA, IgD and IgE, respectively.

In some embodiments, antibodies exist as intact immunoglobulins or as a number of well-known fragments. In some embodiments, pepsin digests an antibody below the disulfide linkages in the hinge region to produce F(ab)′₂, a dimer of Fab which itself is a light chain joined to VH-CH1 by a disulfide bond. In some embodiments, the F(ab)′₂ may be reduced under mild conditions to break the disulfide linkage in the hinge region thereby converting the (Fab′)₂ dimer into Fab′ monomers. In some embodiments, the Fab′ monomer is an Fab with part of the hinge region (see, Fundamental Immunology, W. E. Paul, ed., Raven Press, N.Y. (1993), which is incorporated by reference in its entirety for all purposes). In some embodiments, antibody fragments are synthesized de novo either chemically or by utilizing recombinant DNA methodology. In some embodiments, antibodies include V_{H}—V_{L} dimers, including single chain antibodies (antibodies that exist as a single polypeptide chain), such as diabodies, or single chain Fv antibodies (sFv or scFv) in which a variable heavy and a variable light region are joined together (directly or through a peptide linker) to form a continuous polypeptide. (e.g., Huston, et al. Proc. Nat. Acad. Sci. USA, 85:5879-5883, 1988, which is incorporated by reference in its entirety for all purposes). In some embodiments, antibodies can be another fragment, including, for example, Fab molecules displayed on phage if one of the chains (heavy or light) is fused to g3 capsid protein and the complementary chain exported to the periplasm as a soluble molecule. (e.g., U.S. Pat. No. 5,733,743, which is incorporated by reference in its entirety for all purposes). In some embodiments, the antibody is an scFv antibody or a number of other structures converting the naturally aggregated, but chemically separated light and heavy polypeptide chains from an antibody V region into a molecule that folds into a three dimensional structure substantially similar to the structure of an antigen-binding site are known to those of skill in the art (e.g., U.S. Pat. Nos. 5,091,513, 5,132,405, and 4,956,778, which are all incorporated by reference in their entirety for all purposes). In some embodiments, the scFv is a diabody as described in Holliger et al., Proc. Nat'l Acad. Sci. vol. 90, pp. 6444-6448 (1993), which is incorporated by reference in its entirety for all purposes. In some embodiments, antibodies include all those that have been displayed on phage or generated by recombinant technology using vectors where the chains are secreted as soluble proteins, e.g., scFv, Fv, Fab, pr (Fab′)₂. Antibodies can also include miniantibodies. In some embodiments, the antibody is from a B-cell, a plasma cell, a B memory cell, a pre-B-cell or a progenitor B-cell.

In some embodiments, the immune binding protein is a T-cell receptor. In some embodiments, the T-cell receptor is from a CD8+ T-cell, a CD4+ T-cell, a regulatory T-cell, a memory T-cell, a helper T-cell, or a cytotoxic T-cell. In some embodiments, T-cell receptors are obtained from either (or both) the genomic DNA of the T-cells (or subpopulation of T-cells) and/or the mRNA of the T-cells (or subpopulation of T-cells). In some embodiments, repertoires of T-cell receptors are obtained using techniques and primers well known in the art and described in, for example, SMARTer Human TCR a/b Profiling Kits sold commercially by Clontech, Boria et al., BMC Immunol. 9:50-58 (2008); Moonka et al., J. Immunol. Methods 169:41-51 (1994); Kim et al., PLoS ONE 7:e37338 (2012); Seitz et al., Proc. Natl Acad. Sci. 103:12057-62 (2006), all of which are incorporated by reference in their entirety for all purposes. In some embodiments, the T-cell receptors are used as separate chains to form an immune binding protein. In some embodiments, the T-cell receptors are converted to single chain antigen binding domains. In some embodiments, single chain T-cell receptors are made from nucleic acids encoding human alpha and beta chains using techniques well-known in the art including, for example, those described in U.S. Patent Application Publication No. US2012/0252742, Schodin et al., Mol. Immunol. 33:819-829 (1996); Aggen et al., “Engineering Human Single-Chain T Cell Receptors,” Ph.D. Thesis with the University of Illinois at Urbana-Champaign (2010) a copy of which is found at ideals.illinois.edu/bitstream/handle/2142/18585/Aggen_David.pdf?sequence=1, all of which are incorporated by reference in their entirety for all purposes.

In some embodiments, the immune binding protein is an innate immunity receptor. In some embodiments, natural killer cells, dendritic cells, macrophages, T-cells, and/or B-cells are used to make a NKG receptor binding proteins and/or Toll-like receptor binding proteins. In some embodiments, the natural killer cells, dendritic cells, macrophages, T-cells, and/or B-cells are obtained from a subject who has become immune to a disease or has had an immune response to a disease or condition. In some embodiments, the immune binding proteins is obtained from the CD94/NKG2 receptor family (e.g., NKG2A, NKG2B, NKG2C, NKG2D, NKG2E, NKG2F, NKG2H), the 2B4 receptor, the NKp30, NKp44, NKp46, and NKp80 receptors, the Toll-like receptors (e.g., TLR1, TLR2, TLR3, TLR4, TLR5, TLR6, TLR7, TLR8, TLR9, TLR10, RP105), and/or innate immunity receptors are obtained from the subjects immune cells (natural killer cells, dendritic cells, macrophages, T-cells, and B-cells). In some embodiments, the immune binding proteins of the invention are made as described in U.S. Pat. Nos. 5,359,046, 5,686,281 and 6,103,521 (which are hereby incorporated by reference in their entirety for all purposes). In some embodiments, the immune binding protein is part of a receptor which is monomeric, homodimeric, heterodimeric, or associated with a larger number of proteins in a non-covalent complex. In some embodiments, a multimeric receptor has only one polypeptide chain with a major role in binding to the ligand. In these embodiments, the immune binding protein can be derived from the polypeptide chain that binds the ligand. In some embodiments, the immune binding protein is a complex of extracellular portions from several proteins that forms covalent bonds through disulfide linkages. In some embodiments, the immune binding protein is comprised of truncated portions of a receptor, where such truncated portion is functional for binding ligand.

### Methods for Amplifying Nucleic Acids Encoding Multimeric Immune Proteins

The invention relates to methods for making nucleic acids encoding immune binding proteins that preserve the in vivo multimeric associations of the immune polypeptide chains making up the immune binding protein (e.g., antibodies, T-lymphocyte receptors or innate immunity receptors). In some embodiments, immune binding protein libraries of the invention are enriched for nucleic acids encoding multimers that are functional polypeptides representing the multimeric complexes found in the repertoire from which the immune binding protein library was obtained. In some embodiments, the nucleic acids encoding the polypeptide chains for immune binding proteins are derived from individuals whom have mounted an immune response relevant to, for example, an infectious disease, a cancer, an autoimmune disease, an allergy, or a neurodegenerative disease. In some embodiments, the infectious disease is caused by an influenza virus. In some embodiments, the infectious disease is caused by an infectious agent virus such as, for example, HIV, Ebola, Zika, HSV, RSV, or CMV.

In some embodiments, the immune binding proteins are antibodies or are immune binding proteins derived from antibodies. In some embodiments, the immune binding proteins are T-cell receptors from, for example, cytotoxic T-cells, helper T-cells, and memory T-cells. In some embodiments, the immune binding proteins are innate immune receptors such as, for example the CD94/NKG2 receptor family (e.g., NKG2A, NKG2B, NKG2C, NKG2D, NKG2E, NKG2F, NKG2H), the 2B4 receptor, the NKp30, NKp44, NKp46, and NKp80 receptors, the Toll-like receptors (e.g., TLR1, TLR2, TLR3, TLR4, TLR5, TLR6, TLR7, TLR8, TLR9, TLR10, RP105).

In some embodiments, immune binding proteins are made from individual cells that are placed into microwells and/or an emulsion. In some embodiments, forward (F) and reverse (R) primers are used for each individual chain of the immune binding protein (e.g., heavy (H) and light (L) chain primers designated HF, HR, LF, and LR), as well as a polymerase enzyme and dNTPs to carry out template-directed amplification. In some embodiments, the primers for an individual chain of the immune binding protein (e.g., the HF and HL primers for an antibody heavy chain and/or alternatively the LF and HR primers for the antibody light chain) contain an overlap extension region (OE) such that during cycled amplification the primers for one chain extend (amplify) nucleic acids encoding the other chains of the immune binding protein. In some embodiments, a joint polypeptide (such as a scFv or a single chain T-cell receptor) can be encoded by the amplified nucleic acids, and the OE region can optionally encode an amino acid linker sequence.

In some embodiments, the amplification reaction is carried out, resulting in a nucleic acid which codes for each of the polypeptides from the immune binding protein (e.g., both a heavy and a light chain of an antibody). In some embodiments, the nucleic acid obtained from each well and/or emulsion is homogeneous and encodes the immune binding protein (e.g., antibody) made by the single cell placed in the microwell and/or emulsion. In some embodiments, nucleic acids obtained from the wells and/or emulsions are pooled to form a library of heavy/light chain pairs that reflect the pairing of the antibody chains from the source cells or genetic material.

In some embodiments, the resulting pool of nucleic acids encoding paired heavy and light chains for the antibodies are cloned into an expression vector or can be processed for sequencing. In some embodiments, the expression vector is engineered for phage display, yeast display, or other display technology. In some embodiments, the expression vector is for secretion expression and recombinant production of the antibodies. In some embodiments, the expression vector is for making a library of chimeric antigen receptors, where each CAR has one of the paired antibody clones obtained from the amplification reaction. In some embodiments, primers corresponding to heavy chains or light chains may be targeted to single isotypes of antibodies (e.g., IgG), or pools of primers corresponding to all available isotypes or some fraction thereof may be used.

In some embodiments, primers for the light chain and heavy chain are linked together so that each primer is capable of priming a reaction. In some embodiments, a 5′ azide-alkyne reaction (“Click”) coupling can bring together the heavy and light chain primers. In this embodiment, the dual primer is incubated with single cells in a well or emulsion, and nucleic acids are obtained where a nucleic acid encoding a heavy chain is linked to a nucleic acid encoding the paired light chain. In some embodiments, a microsurface (e.g., bead or microwell) is prepared and contains primer sequences that are capable of binding either heavy or light chain nucleic acids. Following mRNA capture, cDNA synthesis or PCR from a single cell in a spatial confinement with the primers in the well or on the bead, nucleic acids encoding the paired heavy and light chains become co-located with the heavy and light chain primers of the solid phase.

In some embodiments, nucleic acid probes for nucleic acids encoding heavy and light chain polypeptides are placed on a solid surface. In this embodiment, the probes for nucleic acids encoding heavy and light chain antibody polypeptides are interrogated with nucleic acids, e.g., mRNA, from a single cell. The probes on the solid phase will capture paired light and heavy chains encoding nucleic acids from the cell. In some embodiments, captured mRNA is reverse transcribed to make paired cDNAs encoding the light chain and heavy chain polypeptides from a single cell.

In some embodiments, the nucleic acids encoding the subunits of the immune binding protein are bar coded to enable identification of unique molecules. In some embodiments, a solid phase with a cell-specific barcode is made with spatially confined PCR reactions of a plurality of single template molecules containing a linker/adapter primer sequence, a random barcode sequence, and a secondary primer sequence. In some embodiments, a limited dilution of template molecules is used, and the template molecule is linked to a solid phase at very low loading rates to ensure only a single molecule is available as a template at each site. In this embodiment, at least one of the primers in this PCR reaction should be attached to the solid phase. In some embodiments, additional molecules may be added to load additional sites, knowing that previously bound sites are incapable of reacting because they were exhausted during previous rounds of PCR.

In some embodiments, oligonucleotides can be attached at an extremely low loading rate to a surface and beads are flowed over the surface to ensure that each bead binds a single oligonucleotide. In some embodiments, beads are reflowed over the surface without being subjected to the constraints of poissonian loading. In some embodiments, a moderate surface of 100 cm², hundreds of millions of beads can be bound to individual molecules. In some embodiments, each bound bead would be guaranteed to have one and only one template sequence. In some embodiments, each spatially confined site (either a position or well on a patterned surface, or bead in emulsion) will contain the same barcoded DNA in close proximity, whereas other sites will each contain separate barcoded DNA in close proximity originating from other single molecule templates. In some embodiments, single stranded DNA can be generated through the use of a 5′ nuclease or denaturation of the uncoupled second strand. In this embodiment, the secondary primer sequence is available to perform a subsequent barcode extension reaction or can be used directly to capture nucleic acids from single cells. In some embodiments, the bead can be ligated to a sequence containing a linker section and a fully random sequence to serve as a unique molecular identifier, and a tertiary primer sequence. In this embodiment, the tertiary primer sequence is available to perform a subsequent barcode extension reaction or can be used directly to capture nucleic acids from single cells.

In some embodiments, a surface (e.g., glass surface) is selectively silanized and functional alkane or PEG (eg FSL, amino, azide, DBCO, florous group) is attached in an array of spots that are smaller than the size of the bead or diameter of the cells to be captured. In some embodiments, the remaining surface is silanized with passivating silane (e.g., alkane or PEG). Functional sites may be additionally modified with proteins or moieties to capture desired cells or specific types of cells. For example, CD19 can be attached to the surface for the capture of B cells from a cell mixture. Target cells are incubated with the surface at concentrations where a small number of cells are captured at each site. The cells are then non-poisonnianly loaded into the array. In some embodiments, a self-assembling hydrogel is generated on top of each cell, for example, using PEG ×4 dendrimer DBCO and PEG 10 kda azide and a heterobifunctional linkage such as DBCO NHS for initial attachment to the cells or array position. Additional molecules may be incorporated in the hydrogel for capture of desired targets. In some embodiments, Protein G is attached for antibody capture, or poly dT oligonucleotides are attached for mRNA capture. Cells in this matrix may then be incubated with molecules for capture of matrix bound agents and therefore labelled, such as primers, DNA molecules, protein antigens, or antibodies. In some embodiments, a lysis solution is added to the cells on the surface, the cells are lysed, and their contents captured within the hydrogel matrix. In some embodiments, various reagents are flowed over the surface, such as wash buffers to remove reagents from a prior step, whilst maintaining bound RNA. In some embodiments, new reagents for a next step are added in this manner, such as, for example a reverse transcriptase solution containing enzyme and suitable buffer for the synthesis of a cDNA library for each cell. In some embodiments, it may be preferable to replace the non-hydrogel aqueous phase with a hydrocarbon or florous oil phase to prevent diffusion of intracellular or extracellular bound materials out of the matrix.

In some embodiments, the surface is patterned with hydrophilic spots on a hydrophobic or florous background. In this embodiment, droplets will self-assemble on the surface and be ready for subsequent reactions. These droplets may be used to generate hydrogels as well using click chemistry as described above. In some embodiments, the spots are on the order of the size of a cell and single cells can be captured in a nonpoissonian manner. In some embodiments, the spots are much larger than a single cell and capture of single cells occurs in a poissonian fashion. In some embodiments, patterning is random rather than arrayed though this may result in lower loading densities.

In some embodiments, each spot contains a plurality of poly-dt primers with the same 5′ random DNA barcode so that each cell's mRNA can be specifically labelled. In some embodiments, a patterned surface is used to first capture a single bead that is smaller than the cell, but larger than the capture site. For example, a capture site of 1 um combined with a bead size of 2 um. In some embodiments, the beads are functionalized so that they can attach to both a cell and the capture site. For example, the beads can be coated with NHS and DBCO, while the capture sites have an azide. After attachment of beads to the capture site, cells are flowed so that each bead captures a single cell.

Once the cells are arrayed, it may be advantageous to transfer them to a microwell array containing other reagents for additional workup, such as lysis and capture of mRNA to primer coated beads. This enables non-poissonian loading of cells and/or beads to a microwell array.

These techniques can be used to capture single cells for RNA capture on barcoded beads, or to exactly position a single bead at each capture site for additional workup. For example, barcoded cDNA on a bead may be put on the capture array so that a single bead is at each spot. In some embodiments, a PCR reaction may be performed that amplifies the barcoded section of each molecule and amplifies a particular region of a subset of molecules of interest (for instance heavy and light chains), then links the barcode to the particular region of interest via ligation or assembly PCR. In this manner a sequencing read will contain the region of interest and the barcode and not be subject to the barcode being on the 5′ or 3′ ends of a molecule longer than the sequencing read length.

### Methods for Isolating Immune Binding Proteins

Described herein are methods for isolating an immune binding protein, such as antibodies (including light and heavy chains), T-lymphocyte receptors and innate immunity receptors, in combination with the antigen(s) to which these immune proteins binds. The methods set forth herein, describe and allow for the multiplexing a plurality of immune binding proteins with a plurality of antigens such that it is uniquely possible to identify a complete set of specific binding pairs.

### Preparation of Bait Particle(s)

As used herein, “bait particle(s)” of the invention include, for example, magnetic beads, beads having at least one fluorophore or other suitable beads as described herein. Magnetic beads of the invention may include, for example: Dynabeads and Pierce magnetic beads. Suitable fluorophores of the invention may include, for example: UV fluorophores, Red fluorophores, Green fluorophores, Blue fluorophores and Orange fluorophores. In the described methods, antigens of interest are subsequently attached to the uniquely prepared bait particles. Bait particles of the invention may further include an oligonucleotide, such as a sequence primer binding site, a nucleic acid bar code and a primer for target bar code.

Bait particles of the invention, may contain a plurality of different antigens (see for example, Example 32), which illustrates a method for preparing a plurality of bait particles with a plurality of HA antigens from different influenza virus stains/isolates. Briefly, HA acquired from a plurality of different influenza isolates, are mixed with a plurality of targets. The targets in this example, are antibodies secured from a plurality of subjects whom have been immunized with an influenza vaccine from at least some of the influenza isolates. However, in the present invention, suitable antigens may be any antigen from isolated proteins or other macromolecules, cells, cell debris, virus particles or viral components, such as capsids. As described herein, the plurality of targets may be bar coded. For example, antibodies from each unique subject are given a bar code to identify the specific subject, which is the source of the antibodies.

### Isolating Clones of Specific Targets Utilizing Bait Particles and Sequencing

Bait particles of the invention can be employed to isolate specific targets, such as specific cells, which include, for example: B-cells, T-cells, NK cells, innate immunity cells and tumor cells, antibodies, or display library clones, such as antibody or antigen-specific T cell receptor (“TCR”) libraries that bind to the antigen. Optionally, and similarly to the bait particles described herein, the specific targets of the invention may also be bar coded.

Identified binding pairs of bait particles having at least one HA and a target from an individual subject, are isolated together from any unbound target (i.e., antibodies), by separating the bait particles through, for example, a centrifugation spin-down or by magnetic isolation technique. Sequencing preparation can use the bait particles with primers to produce copies of the nucleic acids from the target, for example, by employing a bar code and/or nucleic acid encoding the binding protein in the cell or phage. The primer on the bait particles binds to a target nucleic acid and yields a copy suitable for sequencing.

Subsequently, the collected binding pairs are isolated into specific individual particles and ultimately sequenced to identify the specific antigen (HA isolate) and target (subject from which the antibody was isolated). Alternatively, the sequence information can provide the bait antigen and the nucleic acid sequence of the target binding protein. The sequencing approach can use any platform, including, for example: Roche 454 FLX Titanium and 454 FLX; Illumina HiSeq 1000, HiSeq 1500, HiSeq 2000, HiSeq 2500, HiSeq 3000, HiSeq 4000, HiSeqX ten, NovaSeq5000 and NovaSeq6000; Life Technologies SOLiD 4, SOLiD 5500, SOLiD 5500xl, SOLiD 5500W and SOLiD 5500xlW.

### Gel and Core-Shell Beads for Cell Encapsulation

Although some emulsions are suitable for the isolation of single cells, the ability to manipulate such cellular emulsions through the addition of reagents, buffers, enzymes and other desirable materials, remain difficult and cumbersome. Thus, provided herein, are methods and compositions for simplifying single-cell handling and manipulation, wherein the addition of a reagent, buffer and/or enzyme is required or desired. Advantageously, the methods and compositions described herein permit the manipulation of single cells without a loss of the clonal nature of the cells.

The inventors have discovered a unique method for achieving an enriched or uniform population of encapsulated single cells in a droplet, wherein a gelation reagent and other useful reagents can be introduced to the droplet. Devices and methods for the encapsulation of cells utilizing microfluidic platforms are also disclosed. Useful microfluidic devices of the invention generally include a plurality of functional regions to shear, focus and encapsulate a desired individual cell or group of cells and/or “scaffold,” into a droplet. The microfluidic devices of the invention, are designed such that gelling materials are introduced to a cell containing droplet and is subsequently rapidly polymerized (activated) to form gel beads.

In some embodiments of the invention, droplets are rapidly gelled on a micro-surface, such as a chip, through the manipulation of temperature, chemical stimulation or through light stimulation. Such manipulations are described in further detail below.

In some embodiments, droplets are rapidly gelled on-chip through the manipulation of temperature, chemical stimulation or through light stimulation. Such manipulations are described in further detail below.

In other embodiments, droplets are “semi-stabilized” on a chip to permit for a longer period of time for on-chip gelation through interfacial polymerization. Semi-stabilized techniques are also further detailed below.

In some embodiments, the microfluidic devices of the invention are those having laminar flow (cross-flow channels), As used herein, the term “laminar flow” corresponds to a Reynolds number below 2000, and, in some instances, below 20. Suitable microfluidic devices of the invention are described herein. In some embodiments, a core aqueous fluid containing cells, a gelling agent and other optional reagents described herein, are cross-flowed in a microfluidic device with an oil. The cross-flow of oil forms droplets in a water/oil emulsion. Once the droplets are formed, gelation is induced through manipulation of temperature, chemical stimulation, or through light stimulation. These methods and compositions are described in detail herein.

In other embodiments, the microfluidic devices of the invention are those having multiple cross-flow channels. At a first cross-flow channel a core aqueous fluid containing cells and other optional reagents described herein are cross-flowed in a microfluidic device with an oil. The cross-flow of oil forms droplets in a water/oil emulsion. At a second cross-flow channel, the water/oil droplet from the first cross-flow channel is cross-flowed with a second aqueous fluid containing a gelling agent and other optional reagents described herein. The cross-flow of water/oil droplets and aqueous forms droplets of a water/oil/water emulsion. At a third cross-flow channel, the water/oil/water droplet from the second cross-flow channel is cross-flowed with an oil. The cross-flow of oil forms droplets of a water/oil/water/oil emulsion. Once the droplets are formed, gelation is induced through manipulation of temperature, chemical stimulation, or through light stimulation.

### Microfluidic Devices

Microfluidic systems have been described in a variety of contexts, typically in the context of miniaturized laboratory (e.g., clinical) analysis. Other uses have been described as well. For example, International Patent Application Publication No. WO 01/89788 describes multi-level microfluidic systems that can be used to provide patterns of materials, such as biological materials and cells, on microsurfaces, for example, a chip. Other publications describe microfluidic systems including valves, switches, and other components. The microfluidic devices and methods of use described herein are based on the creation and electrical manipulation of aqueous phase droplets, which can introduce, for example, cells, enzymes and reagents, such as gelation reagents and reagents for molecular retention, and then be encapsulated by an inert oil stream. This combination enables electrically addressable droplet generation, highly efficient droplet coalescence, precision droplet breaking and recharging, and controllable single droplet sorting. Additional passive modules include multi-stream droplet formulations, mixing modules, and precision break-up modules. The integration of these modules is an essential enabling technology for a droplet based, high-throughput microfluidic reactor system.

The microfluidic devices of the present invention can use a flow-focusing geometry to form the droplets. For example, a water stream can be infused from one channel through a narrow constriction; counter propagating oil streams (preferably fluorinated oil) hydrodynamically focus the water stream and stabilize its breakup into micron size droplets as it passes through the constriction. In order to form droplets, the viscous forces applied by the oil to the water stream must overcome the water surface tension. The generation rate, spacing and size of the water droplets is controlled by the relative flow rates of the oil and the water streams and nozzle geometry.

While this emulsification technology is extremely robust, droplet size and rate are tightly coupled to the fluid flow rates and channel dimensions. Moreover, the timing and phase of the droplet production cannot be controlled. To overcome these limitations, the microfluidic devices of the present invention can incorporate integrated electric fields, thereby creating an electrically addressable emulsification system. In one embodiment, this can be achieved by applying high voltage to the aqueous stream and charge the oil water interface. The water stream behaves as a conductor while the oil is an insulator; electrochemical reactions charge the fluid interface like a capacitor. At snap-off, charge on the interface remains on the droplet. The droplet size decreases with increasing field strength. At low applied voltages the electric field has a negligible effect, and droplet formation is driven exclusively by the competition between surface tension and viscous flow, as described above.

The microfluidic, droplet-based reaction-confinement system of the present invention can further include a mixer which combines two or more reagents to initiate a chemical reaction. Multi-component droplets can easily be generated by bringing together streams of materials at the point where droplets are made. However, all but the simplest reactions require multiple steps where new reagents are added during each step. In droplet-based microfluidic devices, this can be best accomplished by combining (i.e. coalescing) different droplets, each containing individual reactants. However, this is particularly difficult to achieve in a microfluidic device because surface tension, surfactant stabilization, and drainage forces all hinder droplet coalescence; moreover, the droplets must cross the stream lines that define their respective flows and must be perfectly synchronized to arrive at a precise location for coalescence. The microfluidic devices of the present invention overcome these difficulties by making use of electrostatic charge, placing charges of opposite sign on each droplet, and applying an electric field to force them to coalesce. By way of non-limiting example, a device according to the present invention can include two separate nozzles that generate droplets with different compositions and opposite charges. The droplets are brought together at the confluence of the two streams. The electrodes used to charge the droplets upon formation also provide the electric field to force the droplets across the stream lines, leading to coalescence. In the absence of an electric field, droplets in the two streams do not in general arrive at the point of confluence at exactly the same time. When they do arrive synchronously the oil layer separating the droplets cannot drain quickly enough to facilitate coalescence and as a result the droplets do not coalesce. In contrast, upon application of an electric field, droplet formation becomes exactly synchronized, ensuring that droplets each reach the point of confluence simultaneously (i.e., paired droplets).

Of particular interest in the present invention, are microfluidic devices capable of encapsulating single cells in droplets formed by water/oil emulsions (“W/O”). Such devices include, for example, but are not limited to devices that employ Electrokinetic Mechanisms (Electrical forces for microscale cell manipulation. Voldman J, Annu Rev Biomed Eng., 80:425-54 (2006)); Harnessing dielectric forces for separations of cells, fine particles and macromolecules, Gonzalez et al., J Chromatogr A., 1079(1-2):59-68 (June 2005)); Dielectrophoresis, which, in contrast to electrophoresis, where cells move in a uniform electric field due to their surface charge, dielectrophoresis (“DEP”) refers to the movement of cells in a non-uniform electric field due to their polarizability. For movement in response to a dielectrophoretic force, cells do not need to possess a surface charge because, unlike a DC field, an alternating current (AC) is capable of polarizing the cell (i.e., inducing a dipole moment across the cell) (Electrical forces for microscale cell manipulation, Voldman J. Annu Rev Biomed Eng., 80:425-54 (2006)); and Acoustophoresis, which refers to the movement of an object in response to an acoustic pressure wave. Recently, acoustic microfluidic (i.e., acoustofluidic) technologies have provided many new areas of development within analytical flow cytometry, including the sorting of cells (Austin Suthanthiraraj P P et al., Methods., 57:259-271 (2012)). Acoustic forces are amenable to cell handling as they can provide rapid and precise spatial control in microchips without affecting cellular viability (Lenshof et al., Chemical Society reviews., 39:1203-1217 (2010); Lenshof et al., Lab Chip., 12:1210-1223 (2012); Burguillos et al., PloS one., 2013; 8:e64233 (2013); Laurell et al., Chemical Society reviews., 36:492-506 (2007)). In this context, acoustic waves can be divided into three categories: bulk standing waves (Johansson et al., Analytical chemistry, 81:5188-5196 (2009)); standing surface acoustic waves (SSAWs) (Ding X et al., Lab Chip, 13:3626-3649 (2013); and traveling waves (Cho S H et al., Lab Chip, 10:1567-1573 (2010).

In some embodiments of the invention, core-shell gel beads can be prepared through either the microfluidic methods described herein, or by specific reagent methods. Examples of microfluidic methods useful in the present invention include, but are not limited to: co-axial flow in non-nested channels; geometric confinement in non-nested channels; double and higher order emulsions. An example of a reagent method includes, but is not limited to, an aqueous two-phase system (“ATPS”). ATPSs are typically characterized by having two immiscible aqueous phases and have traditionally been used for the separation and purification of biological material such as proteins or cells. Microfluidic implementations of such schemes are usually based on a number of co-flowing streams of immiscible phases in a microchannel, thereby replacing the standard batch by flow-through processes. Some aspects of the stability of such flow patterns and the recovery of the phases at the channel exit are reviewed. Furthermore, the diffusive mass transfer and sample partitioning between the phases are discussed, and corresponding applications are highlighted. When diffusion is superposed by an applied electric field normal to the liquid/liquid interface, the transport processes are accelerated, and under specific conditions the interface acts as a size-selective filter for molecules. Finally, the activities involving droplet microflows of ATPSs are reviewed. By either forming ATPS droplets in an organic phase or a droplet of one aqueous phase inside the other, a range of applications has been demonstrated, extending from separation/purification schemes to the patterning of surfaces covered with cells.

Electrophoresis.

Electrophoresis refers to the movement of suspended particles toward an oppositely charged electrode in direct current (DC). Since most cells possess a slight negative charge due to a locus of chemical groups on their surface, they migrate toward the positive electrode during electrophoresis, and the electrophoretic force exerted on that cell is proportional to its charge (Voldman J., Annual review of biomedical engineering, 8:425-454 (2006)). Takahashi et al. applied electrophoresis to sort cells in a microchip in which an upstream fluorescence detector identified labeled cells for rapid electrostatic sorting downstream (Takahashi K et al., Journal of nanobiotechnology, 2 (2004)). Yao et al. developed a similar device based on gravity that operated in an upright orientation to process cells without convective flow (Yao B et al., Lab on a Chip, 4:603-607 (2004)). A more recent example by Guo et al. showed electrophoretic sorting with much higher throughputs by sorting water-in-oil droplets under continuous flow (Guo F et al., Applied Physics Letters, 96:193701 (2010)). In this system, prefocused cells were encapsulated into droplets such that droplets containing single cells were sorted from droplets containing no cells or multiple cells.

Dielectrophoresis (“DEP”).

In contrast to directly sorting cells in a buffered suspension, several groups have developed systems to encapsulate single cells into emulsified droplets for sorting using DEP, thus enabling continuous genomic and proteomic analyses downstream (Baret J C et al., Lab Chip, 9:1850-1858 (2009); Agresti J J et al., Proceedings of the National Academy of Sciences of the United States of America, 107:4004-4009 (2010); Mazutis L et al., Nature protocols, 8:870-891 (2013)). Unlike FACS, which can generate potentially biohazardous aerosols, water-in-oil droplets provide a safe and rapid way to analyze individual cells post-sorting. Baret et al. applied DEP in a fluorescence-activated droplet sorter to separate up to 2,000 cells/sec. Agresti et al. used emulsions to generate picoliter-volume reaction vessels for detecting new variants of molecular enzymes and dielectrophoretic sorting. Mazutis et al. showed that cells compartmentalized into emulsions with beads coated with capture antibodies can be used to analyze the secretion of antibodies from cells for downstream sorting using DEP. These advances may also enable clinical detection, analysis, and diagnosis using a single microchip.

Standing Surface Acoustic Waves (“SSAW”).

In contrast to bulk acoustic standing waves, SSAW devices form a standing wave along the floor of the microfluidic channel using interdigital transducers (IDTs), providing the mechanical perturbations necessary to position cells along well-defined flow streams in the fluid above (Shi J et al., Lab Chip, 9:3354-3359 (2009)). SSAW devices show particular promise for fluorescent label-based cell sorting since a single device can provide a large range of frequencies for dexterous spatial control of single cells and, in turn, multiple channels for sorting (Wang Z et al., Lab Chip, 11:1280-1285 (2011); Lin S C et al., Lab Chip, 12:2766-2770 (2012)). These devices have efficiently sorted cells in buffer as well as in water-in-oil droplets across five fluidic channels (Ding X et al., Lab Chip, 12:4228-4231 (2012); Li S et al., Analytical chemistry, 85:5468-5474 (2013)). Ding et al. further showed that SSAW devices can function as acoustic tweezers to manipulate the spatial orientation and patterning of cells and whole organisms such as C. elegans (Ding X et al., Proceedings of the National Academy of Sciences of the United States of America, 109:11105-11109 (2012)).

Employing any of the microfluidic devices and methods described above, or those known in the art, to encapsulate single cells in droplets formed by W/O emulsions, additional manipulated can be achieved as described below.

### High-Throughput System (“HTS”) for Single-Cell and Single Particle Isolation

The HTS device described herein, (“SingleCyte™ device”), is capable of analyzing a large and diverse population of cells and/or particles, followed by the isolation of single cells and/or particles from the population of cells and/or particles. Selection criteria include the isolation of not only the cell or particle itself, but also any secreted products, for example, antibodies, cytokines and/or metabolites secreted by a cell. The SingleCyte™ device allows for the isolation of specific and individual cells and/or particles from a heterogeneous population of cells and/or particles, and, unlike current devices known in the art, uniquely includes the precision required for single cell and/or particle isolation, for example, by using high-resolution handling in the Z axis and in the XY source stage, while still allowing for high-speed manipulation in the XY axis for the destination stage. Additionally, the SingleCyte™ device can work in concert with existing platforms for studying single cell activity and genetic analysis. Specifically, the device may be adapted to work with many microscopy-based or fluorescent assays, and the output of the system is also compatible with standard library preparation techniques.

The HTS device can be used to identify and study single cells of the immune repertoire; e.g., to identify cells expressing immune proteins with specific reaction to antigens of interest. The HTS device also may be utilized to analyze a tumor microenvironment, where different cell types may enhance or inhibit a tumor response. The HTS device may be capable of increasing recombinant protein production where high yield clones can be isolated through precise clonal selection. As detailed herein, the SingleCyte™ device represents a significant advancement over current technologies known in the art.

In addition to the advantages cited above, the SingleCyte™ device is further capable of supplementing existing platforms for interrogating cell activity and performing genetic analysis. For example, the HTS device may be employed to further augment instrumentation for cell isolation, such as flow cytometry, instrumentation for automating single-cell genomic protocols, such as liquid handlers for barcoding, and consumables and reagents. Uniquely, the device permits greater flexibility by uncoupling functions such as cellular isolation and genomic processing typically utilized in the art.

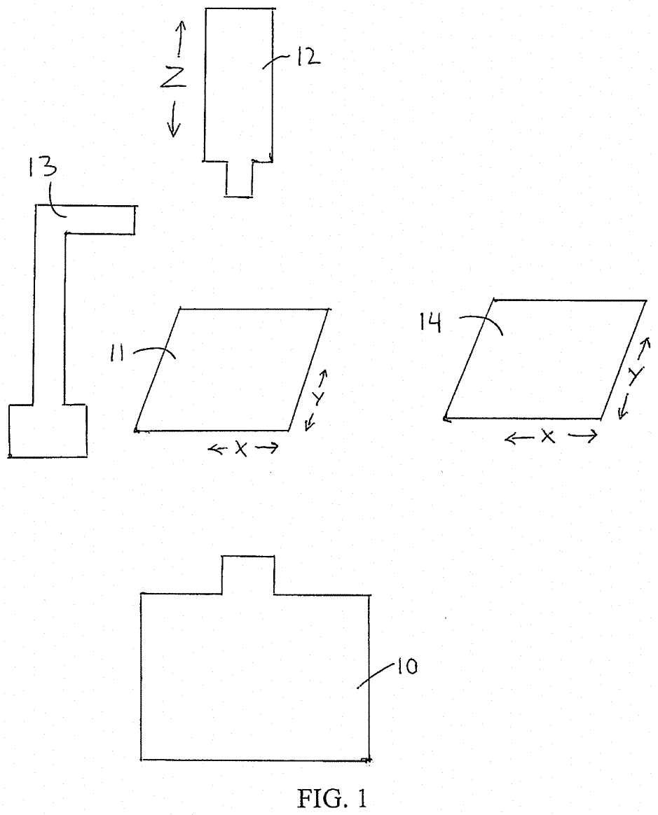

In one aspect, a high-throughput single-cell picking device is provided as shown in FIG. 1. This device is generally characterized by having five (5) primary components. Briefly, and as shown in FIG. 1, 10 represents an inverted microscope and camera component. 11 represents a source substrate component, 12 is a cell picker component 13 is a robotic arm component, and 14 is a destination component. The inverted microscope and cameral component can be a Zeiss Axiovert 200M inverted microscope (Carl Zeiss AG). The inverted microscope and camera component may also be a Nikon, Olympus or Leica capable of similar functions. The inverted microscope and camera component can be employed to map the location of cells (“cellular mapping”) on a substrate component 11, which may be a culture dish, slide, or a microplate or microwell plate, such that a single-cell picker 12 may collect the cells. A robotic arm component 13, can be employed to move the cell picking component 12 to the correct position on a destination component (or alternatively to move the destination component toward the cell picker component 12) (as shown in FIG. 1).

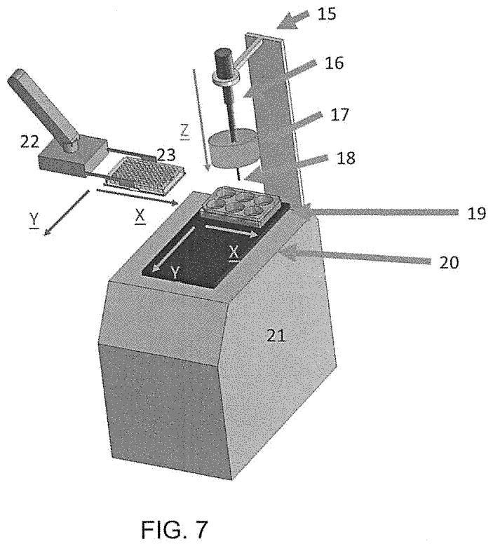

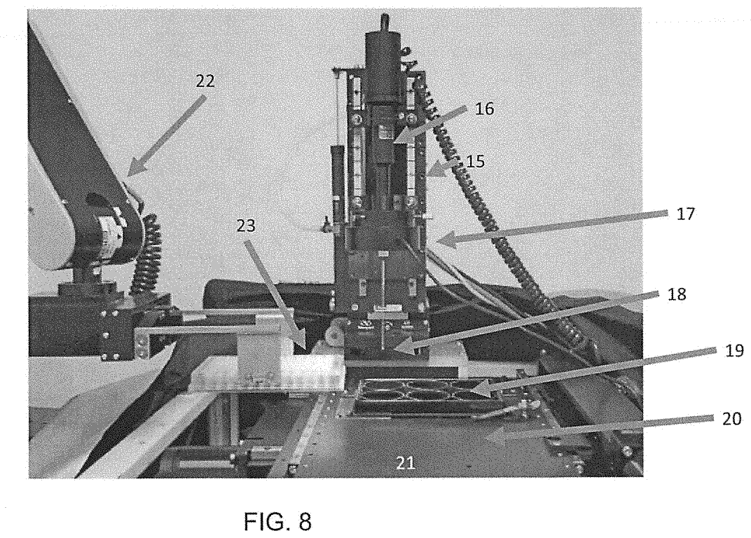

In another aspect, alternative high-throughput single-cell picking devices are provided as shown in FIGS. 7 and 8. These devices are generally characterized by having nine (9) components. Briefly, and as shown in FIGS. 7 and 8, 15 represents a precision Z-axis stage, 16 represents a micropipette, 17 is a ring light, 18 is 50 micron I.D. tip, 19 is a source plate with cells and/or particles (eg., beads), 20 is a robotic gantry for moving the source plate, 21 is the microscope body, 22 is a robot arm or gantry for the receiving substrate, and 23 is the receiving substrate for the individual cells and/or particles. The microscope can be a Zeiss Axiovert 200M inverted microscope (Carl Zeiss AG). The microscope may also be a Nikon, Olympus or Leica capable of similar functions. The microscope can be employed to map the location of cells (“cellular mapping”) on a source plate 19, which may be a culture dish, slide, or a microplate or microwell plate, such that a micropipette 16 may collect the cells.

Cellular mapping may include selection criteria, for example, a cutoff for an optical signal from the cells, which may include, for example, labeling of the cells or production of a reporter in the cells that may include a temporal component. In this embodiment, only cells above a certain optical signal threshold are mapped and picked by cell picker 12 for deposition in a microwell plate positioned on substrate component 14. Robotic arm component 13, in one embodiment, is a ThermoScience® CRS CataLyst Express robot handler (Thermo Fisher Scientific). Other components that provide similar function for robotic arm 13 include, for example, arms made by Agilent Inc., Peak Analysis and Automation (“PAA”), Retisoft, Inc. and Tecan, Inc.

Cell picker component 12 is employed to select and remove cells from substrate component 11 and transfers these selected cells to another substrate component. In this embodiment, the substrate component is a substrate having microwells, for example, a 96-well plate or other suitable consumable as detailed herein. Cell picker 12 utilizes precise incremental movement control and is able to move preferably between about 0.1 μm to about 10 μm, more preferably between about 0.5 μm to about 5 μm, and most preferably between about 1 μm to about 3 μm within a cell in the Z axis as indicated by FIG. 1. Similarly, robotic arm component 13 is able to move substrate component 11 along the X-Y axes between about 1 μm to about 200 μm, more preferably between about 10 μm to about 150 μm, and most preferably between about 50 μm to about 100 μm as indicated by FIG. 1.

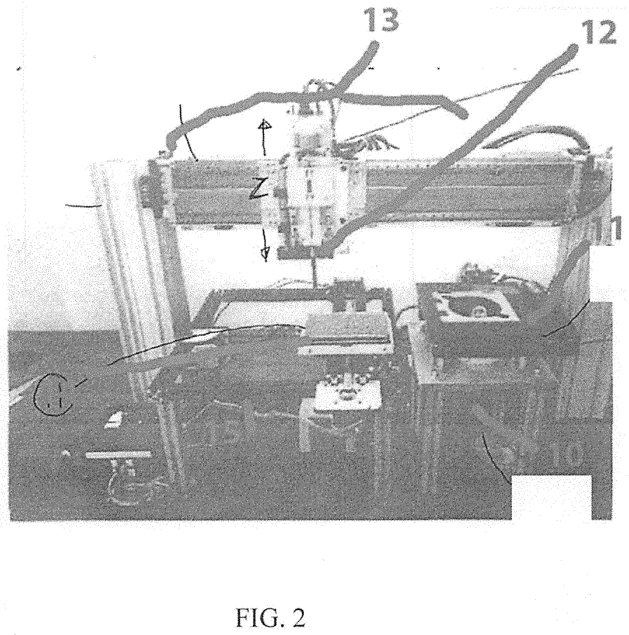

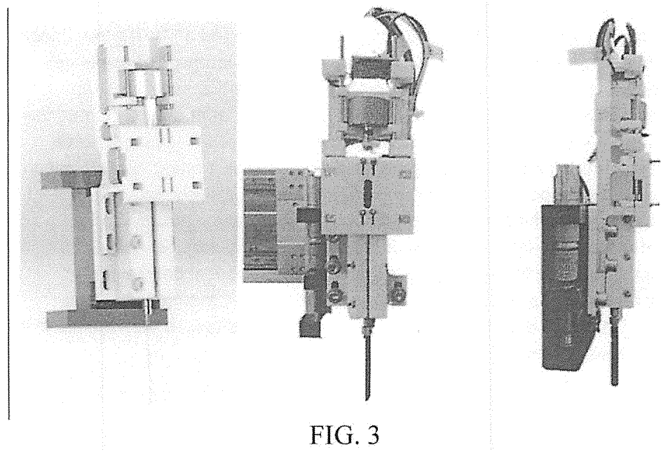

A high-throughput, single-cell picking device can be provided as shown in FIG. 2. The device includes previously described device components, 10, 11, 12, 13, and 14 and further includes X-Y gantry component 15. Substrate component can be fixed to stage component 11. Stage component 11 shown in FIG. 2 is an ASI stage and controller. Additional suitable stage components are available from, for example, Applied Scientific Instruments and include the MS-2000 and LX-4000 controller. In this embodiment, the robotic component providing transfer between the source plate and the destination plate is an IKO linear motor table 13 combined with an XY gantry adapted from a Makerfarm Pegasus 3D printer, consisting of a rolling mount for the destination plate controlled by a pair of stepper motors. In this embodiment, stage component 11 may also include a mount component as shown in FIG. 3. Additionally, Cell-picker component 12 may also include a glass-capillary component as depicted in FIG. 3 for picking individual cells.

### Imaging and Isolating Individual Cells.

Inverted microscope and camera component 10 can be employed to map the location of cells on substrate 11 such that cell-picker 12 identifies and collects the cells. This process is shown in FIGS. 1 and 2. FIG. 2 illustrates stage 11 on which can be placed a plate containing individual cells. Stage 11, as shown in FIGS. 1 and 2, is moveable along the X-Y axes. The mapping of cells may include specific selection criteria, for example, a cutoff value for an optical signal from the cells. This may be accomplished, for example, by labeling of the cells or production of a reporter in the cells that may include a temporal component. In this embodiment, only cells above the optical signal threshold are mapped and picked for deposition into substrate 14. Optionally, substrate on stage 11 may be coated with a hydrogel, for instance a PEG, agarose, acrylamide, or ECM matrix (eg, Matrigel by Corning). Optionally the substrate may be pre-patterned to hold cells in an array. Optionally the substrate may include binding moieties for the capture of biomolecules. Optionally the substrate may be patterned with biomolecules before cells are introduced. Stages 11 and 15 may also be designed to have a “handshake” with their respective plates along the X-Y axes. This handshake may be, for example, a bar code or other set of symbols or words which must be read (recognized) before the apparatus can operate with the substrate. For example, software controlling aspects of the operation can include a step where the handshake (e.g., bar code, symbols, words) are recognized before next steps are performed. If the handshake is not read/found, the apparatus can reject the substrates and cease operation.

Cell-picker component 12 may include an aspiration component for picking up cells. Multiple cells may be introduced into cell-picker component and separated by air or a solution. This embodiment enables cell-picker 12 to quickly select multiple cells from substrate 11 and subsequently deliver single cells to a second substrate by employing multiple aspirations. The cell-picker in this embodiment may employ a sensor to detect the number of cells picked. The sensor can be a coulter counter type sensor or a sensor that interacts with a laser to measure scatter to further characterize the cells. Alternatively, cell sensing may be accomplished employing a device capable of pressure sensing, capacitive sensing or fluidic systems for kinetic assays and multiplexing labeled targets. For example, a pressure signal would increase due to the presence of a cell moving through an aperture in a fluid stream (like a clog). Signal processing and thresholding can be used to determine whether a cell was aspirated. For capacitive sensing, material insertion between 2 capacitive plates will provide a differential signal (increase or decrease in electrical signal depending on the circuit setup). Cells will have different relative permittivity than the fluid medium so when a cell moves between the plates it alters the capacitance between the plates.

The cell-picker component may include a combination positive displacement and pneumatic valve apparatus for high speed droplet ejection from the tip. In this embodiment a syringe apparatus enables the cell picker to aspirate one or more cells, separated by air or solution. A valve at the end of the tip closes the main section of the tip to create an “ejection volume”. The “ejection volume” section of the tip is pressurized at high speed with a pneumatic or mechanical actuator to eject a drop containing a cell or bead.

The SingleCyte™ device may also employ a variety of software programs for image analysis, in addition to adjusting and confirming positioning of substrate on stage 11 and cell-picker 12 for auto-focusing, accuracy and auto-calibration. A variety and combination of selection algorithms for cells may be employed to determine, for example, fluorescence at a single time-point or through temporal observation after a sequential challenge or with a time lapse. The software may also discriminate between positive cells and negative cells like dead cells or unhealthy cells.

Consumables can include, for example, unique substrates and/or microwell plates. For instance a substrate pre-patterned to capture cells in an array with a geometry that is suitable for the picking apparatus. Optionally, a bar code, words (e.g., a poem), or other identifier may be included on individual substrates as a handshake between the substrate and the apparatus. Additionally, cell picker 12 may include a pipette tip composed of an outer large tube that interfaces with the syringe barrel at one end of the picker and a nested, inner tube, preferably made of a flexible material that is used for cell picking. See, for example, FIG. 3.

### Assays Employing the SingleCyte™ Device

### Production of Antibodies

Antibody producing cells, such as B-cells, plasma cells, cells using display technologies, T-cells, or cells with T-cell receptor cells (“TCR cells”), or cells with display technologies for T-cell receptors, or CAR cells, are placed in substrate component on stage 11. Substrate on stage 11 can be a single-cell microwell plate. An antigen with a fluorescent reporter, or other optical reporter can be added to the wells of the microwell plate and washed to remove unbound antigen. Optionally, multiple antigens may be employed, which include, for example, flu isotypes having multiple reporter labels. Screening is designed to identify cells in the wells of the microwell plate that have multiple reporter signals. Alternatively, it is possible to isolate multiple antibody clones against multiple targets in a multiplex reaction. Alternatively, a competition assay may be utilized wherein a known antibody (or known binding protein) is added with the antigen. This process has the ability to identify new binding protein clones having a binding affinity comparable to or higher than the known antibody or known binding protein. In an aspect, the B-cells, plasma cells, cells using display technologies, T-cells, or cells with T-cell receptor cells (“TCR cells”), or cells with display technologies for T-cell receptors, or CAR cells are screened for binding to antigens such as influenza hemagglutinin, influenza NB protein, influenza neuraminidase, SARS-CoV spike protein, coronavirus, herpes virus, HSV gD protein, HSV gG protein, and/or influenza virus.

### Cellular Secretion of Recombinant Proteins

Cells capable of secreting a recombinant protein, or in some examples, non-recombinant protein secreting cells, are placed in substrate on stage 11. In this instance, the substrate on stage 11 is a single-cell microwell array made with glass. The wells of the microwell plate may include a hydrogel or other surface such as glass, titania, ferrous or polymeric microspheres that can capture and/or bind to the secreted proteins, or the wells can be coated in a manner to capture secreted proteins from cells. A bead, such as a magnetic bead, may be placed in the well near the cell to bind the secreted proteins. A second cell “target cell” may be placed in the well to bind the secreted proteins. Additionally, an antigen, ligand or other target can be labeled with a reporter, such as a fluorescent reporter or other appropriate reporter. The wells of the microwell plate can be washed, and subsequently screened to identify cells that make a protein which binds to the antigen, ligand or other target. Wells with a cell making a protein that binds the target can be characterized by a fluorescent signal where the secreted protein is captured and binds a target. For instance, this fluorescent signal could be a halo around the cell where secreted proteins bind to a hydrogel or the surface of the microwell that has been coated with target moieties and counterstained with a secondary reporter that binds the secreted protein, or cells or beads that light up in response to binding secreted protein and a complementary secondary fluorescent reporter. Alternatively the fluorescent signal could be the result of a cell secreting and capturing a protein on its surface after which the secreted protein is stained with a secondary fluorescent reporter. Alternatively, the assay may also be performed in a multiplex format and/or can be done as a competition assay. In another alternative, the wells of the microplate well may be coated with a target, such as an antigen, and individual cells can be subsequently added to the microwells. Antibodies or other binding proteins are secreted and are capable of binding to the antigen. After the wells of the microplate are washed, antibody or other protein bound to the target can be detected. Detection of bound antibody (or other protein) to the target antigen may be accomplished by routine methods known in the art. For example, via a secondary antibody, such as a goat anti-mouse antibody.

### Virus Neutralization Assay.

Target cells can be designated to be infected, and are added to a microwell plate serving as substrate on stage 11. Individual immune cells, such as plasma cells or T-cells, are also added to the wells of the microwell plate. A pre-selected virus strain is subsequently added to the wells and the cells are screened for rescue of target cells via the added cell. Alternatively, sequential challenges may be performed utilizing different virus strains, such as different flu strains. This process may be performed to screen for cells that produce immunity to more than one viral serotype.

### Kinetic Selection

This example is similar to the Production of Antibodies and Cellular Secretion of Recombinant Proteins example described above. Individual cells are placed onto a substrate or into wells. A labeled-target solution is introduced to the wells of the microwell plate, or flowed directly over a substrate, and binding of target, via a reporter signal, is screened over a period of time. This process advantageously can provide affinity information beyond just a yes or no binding response and can aide in the identification of higher affinity candidates from a library of potential candidates.

### Enzymatic Activity.

Enzyme screening may be performed on arrays of cells producing enzymes (e.g., a library of variants or a library of potential candidates) using suitable embodiments described herein and as set forth above. Specifically, a substrate (that generates an optical signal after enzyme activity) for the enzyme of interest is added to each cell in the array (e.g., to each well or flowed over the substrate) and an optical signal is identified. Individual cells that produce signal can then be selected as having candidate enzymes. For instance proteases, lipases, cellulases, amylases and other cleavage enzymes can be screened by introducing a fluorescent substrate bound to a solid surface such that secreted enzymatic activity releases the bound fluorescent moiety. Cells that produce the enzymatic cleavage activity are identified by the absence of spatially confined fluorescence. Multiple substrates may be included with different fluorescent signals or present on different sized particles or different cell types to perform the assay in multiplex.

### Degranulation

In yet another embodiment, activation of specific cells, such as mast cells, eosinophils, basophils (e.g. basophil activation test) or other suitable cells, may be assayed in an array of single cells (e.g., single cells in the wells of a 1-100 micron-scale well array) in response to stimulation through a variety of antibody/antigen interactions. This is accomplished, by co-localizing individual antibody secreting cells with mast cells (or eosinophils or basophils) on the substrate. Reagents are added to the wells to detect the release of components from the granules of mast cells, eosinophils, and/or basophils (e.g., using a labeled antibody that binds the component). This embodiment can analyze the blood/plasma of a subject to identify certain antibodies produced by the immune system, and in addition can identify allergies of a subject.

### Cell Line Engineering

The ability to make multi-component cell arrays can also be useful for identifying candidate production clones from cells engineered to recombinantly produce a product. In this example, cells making the product of interest are placed onto the substrate and then screened for production of the product of interest. Cells which make large amounts of the product (and so have a high signal in the screening) can be identified and cloned. The product produced by the cells can include, for example, recombinant proteins. This assay may rely on the interaction between multiple cells in the array, such that a secreted product from one cell interacts with a second cell to ultimately to generate a signal. For instance a cell containing a partial metabolic pathway (eg, succinic acid production) is introduced with another cell containing a complementary metabolic pathway (eg, nitrogen fixation) and a cell containing a reporter assay that is dependent on the output of the complete metabolic pathway (eg. GFP production).

### RNA Screening and Drug-Genotype Screening

Inhibitory RNAs, such as miRNA, siRNA and antisense RNA, can be employed to prepare a “pseudo-knock out” library of cells. RNAs targeting the genes of interest can be introduced into cells creating a library of cells that have certain genes of interest knocked out (single or multiple knockouts can be in each cell). This library may be subsequently separated into individual/single cells and screened against a target and/or a drug to characterize the effect and impact of the prepared knock outs on the interaction of the cells with the drug.

### TCR/CAR Assay

A cellular library may be prepared by combining a ScFv displayed in a CAR format or a T-cell receptor, with an intracellular signaling pathway responsive to the CAR or TCR binding, and a fluorescent reporter, or other suitable reporter, which is expressed by a promoter that responds to the same intracellular signaling pathway. For example, the calcineurin/NFAT signaling pathway can be used with a CAR. Cells with these components are placed onto the substrate and subsequently exposed to an antigen, which may be present on a cell. The CAR/TCR producing cells are screened to identify those cells that have become activated through by fluorescence and selected by the apparatus.

### Antigen Identification Assay

A library of cells, possibly expressing a library of antigenic peptides/peptides can be introduced onto the substrate. Cells containing a TCR or CAR with an intracellular signaling pathway expressing a fluorescent reporter upon activation can be co-introduced to the array such that each well contains approximately 1 CAR/TCR cell and 1 antigen presenting cells. Upon activation the device is used to select antigen presenting cells that are co-located with fluorescing TCR/CAR cells. cDNA libraries can be prepared from the antigen presenting cells, possibly also containing the TCR/CAR cells, and sequenced to determine the surface proteins responsible for TCR/CAR binding and optionally the sequence of the immune receptor present on the activated TCR/CAR cells.

### ADCC/ADCP Assay

A library of antibody secreting cells can be introduced onto the substrate such that many single antibody secreting cells are individually co-located with a macrophage and/or NK cell. Target cells can be introduced to identify antibody secreting cells that elicit ADCC/ADCP activity. ADCC/ADCP effector function can be determined by target cell death (visualized by morphology changes in microscopy or a live/dead fluorescent stain), reduction in target cell growth, target cell engulfment, or enzyme release of apoptotic cells (e.g., KDalert GAPDH assay from ThermoFisher). Single antibody secreting cells can be selected using the apparatus and placed into conventional 96 well plates for cDNA synthesis and sequencing.

### Bead Library Screening

A library of antigens present on beads, optionally spectrally encoded or DNA barcoded, can be used to pan against a library of phage expressing a surface bound ScFv. The library of antigens bound to ScFv's can be counterstained with an anti-phage fluorescent secondary antibody and loaded onto the substrate. Beads exhibiting fluorescent secondary signal can be selected by the apparatus.

### Antibody Affinity Measurements

A library of antibodies present on beads, with the beads spectrally encoded or ultimately having attached nucleic acid sequences that correspond to its attached antibody subset, where each bead has a generally distinct subset of antibodies, can be loaded onto the substrate. A solution of fluorescent antigens, optionally attached to DNA barcodes, can be flowed over the array in which the concentration of antigen increases over time. The fluorescent signal of the antibody beads can be monitored with respect to the concentration of antibody. Optionally, spectral information on the bead can be used to determine the attached antibody subset. Optionally, individual beads attached to high affinity antibodies can be selected by the apparatus and nucleic acids of the attached antigen/DNA barcode conjugate are amplified and sequenced to determine the attached antigens. Optionally the antibody subset can be determined by amplifying and sequencing the DNA barcode present on the antibody bead.

### In Situ Sequencing and Selection

Nucleic acid molecules present on beads can be sequenced in situ on the device through sequencing by hybridization or sequencing by synthesis so that the sequence of a nucleic acid molecule on a plurality of beads on the substrate is known. Beads of interest can be selected by the apparatus and placed in an external microwell plate.

### Agglutination Assays

Protein binding can be detected via agglutination of quantum dots, beads, cells or polymers. For instance a cell secreting an antibody protein binds to beads with anti-Fc or protein A/G. The beads agglutinate via a sandwich interaction a solution phase secondary protein. The signal can be read out in brightfield, phase, via dynamic light scattering, or fluorescence (including TRF or FRET or dye quenching) depending on the choice of cells, beads or quantum dots. Alternatively the absence of agglutination is used to detect activity of a protein. For instance, cells, such as turkey red blood cells, agglutinate in the presence of HA antigen on the surface of influenza virus. Turkey red blood cells proximal to an antibody secreting cell that obfuscates the binding site of HA antigen to sialic acid do not agglutinate and this signal is used to identify antibody secreting cells with anti-influenza activity. Cells secreting proteins (eg. Antibodies) that cause polymer agglutination (eg, antigen bound to PEG) may also be detected by washing the substrate with a dye molecule and comparing the diffusion rate of the dye near the protein secreting cell.

### Gelation Reagents for Formation of Gel Beads and Shell-Core Beads

Gelation reagents suitable in present invention include those reagents/materials capable of modifying each droplet into a gel having properties sufficient to retain to retain cells and cellular material when the emulsion is broken and the beads are recovered as gel-beads. A selected gelation reagent should be biocompatible and create a pore size within a suitable range. For example, pore sizes between about 1 nanometer (nm) and about 10 nm are typically considered to be small pore sizes, whereas pore sizes in the range of about 100 nm to about 1 micron (μ) are considered to be a large pore size. Typically, the larger the pore size the weaker the gel and the greater the crosslinking the stronger the gel. Thus, the gelation reagents useful in the invention, are those agents which provide sufficient rigidity and strength to undergo later manipulations as described herein. Gelation reagents useful in the instant invention are capable of forming a gel-shell with a liquid core while maintaining compatibility with cell culture and molecular biology processes. Optionally, the composition of the gel-shell can be modified to create a natural barrier capable of retaining or excluding materials based on size or charge.

As used herein, the term “gel” refers to a dilute network of cross-linked material that exhibits no flow when in the steady-state. A “hydrogel” is a gel in which the liquid component of the gel is water. Gels and hydrogels can be deformable. Gels and hydrogels can be in a sol (liquid) or gel (solid) form. In some cases, hydrogels are reversible. Reversible hydrogels can be reversibly transitioned between a sol (liquid—also referred to herein as a “pre-gel”) or gel (solid) form. For example, agarose hydrogel can be transitioned into a sol form with heat and a gel form with cooling. Alternatively, some hydrogel compositions exist in a sol form below a transition temperature and a gel form above the transition temperature. In some cases, a sol (liquid) hydrogel, or hydrogel precursor, can be irreversibly hardened into a gel form. For example, acrylamide can be irreversibly polymerized into a gel form. As used herein, sol refers to either the soluble form of a hydrogel, or soluble hydrogel precursor, and gel refers to a solid hydrogel. Numerous reversible and irreversible hydrogel compositions are known in the art, including those described in, e.g., U.S. Pat. Nos. 4,438,258; 6,534,083; 8,008,476; 8,329,763; U.S. Patent Appl. Nos. 2002/0,009,591; 2013/0,022,569; 2013/0,034,592; and international Patent Publication Nos. WO/1997/030092; and WO/2001/049240.

The term “droplet” refers to a small volume of liquid, typically with a spherical shape, encapsulated by an immiscible fluid, such as a continuous phase or carrier liquid of an emulsion. In some embodiments, the volume of a droplet, and/or the average volume of droplets in an emulsion is, for example, less than about one microliter, such as a “microdroplet,” or between about one microliter and one nanoliter or between about one microliter and one picoliter, less than about one nanoliter (or between about one nanoliter and one picoliter), or less than about one picoliter (or between about one picoliter and one femtoliter), among others. In some embodiments, a droplet (or droplets of an emulsion) has a diameter (or an average diameter) of less than about 1000, 100, or 10 micrometers, or of about 1000 to 10 micrometers, among others. A droplet can be spherical or nonspherical.

The terms “about” and “approximately equal” are used herein to modify a numerical value and indicate a defined range around that value. If “X” is the value, “about X” or “approximately equal to X” generally indicates a value from 0.90× to 1.10×. Any reference to “about X” indicates at least the values X, 0.90×0.91×, 0.92×, 0.93×, 0.94, 0.95×, 0.96×, 0.97×, 0.98%, 0.99×, 1.01×, 1.02×, 1.03×, 1.04×, 1.05×, 1.06×, 1.07×, 1.08×, 1.09×, and 1.10×. Thus, “about X” is intended to disclose, e.g., “0.98×.” When “about” is applied to the beginning of a numerical range, it applies to both ends of the range. Thus, “from about 6 to 8.5” is equivalent to “from about 6 to about 8.5.” When “about” is applied to the first value of a set of values, it applies to all values in that set. Thus, “about 7, 9, or 11%” is equivalent to “about 7%, about 9%, or about 11%.”

Gel beads of the invention can be prepared by manipulating cells contained in single droplets or a plurality of droplets. A gel is created through introduction of a “gelation reagent” material, which captures the cell droplets and permits further introduction of additional materials, such as, but not limited to: buffers, enzymes and reagents. Gelation reagents of the invention include, but are not limited to polysaccharides and proteins, including agarose, alginate, polyacrylamide (poly(2-propenamide) or poly(1-carbamoylethylene, carrageenan, PEG, chitosan, gellan gum, hyaluronic acid, collagen, elastin, gelatin, fibrin and silk fibroin (Gasperini et al., Natural polymers for the microencapsulation of cells, J R Soc Interface, 11(100): 20140817 (November 2014) doi:10.1098/rsif.2014.0817. Gelling reagents of particular interest in the present invention are described more fully below.

Alginates.

In some embodiments of the invention, an alginate is a preferred gelation reagent. An alginate is a polysaccharide, a polyanionic linear block copolymer containing blocks of (1,4)-linked β-D-mannuroic (M block) and α-L-guluronic (G block) acids (Rowley J A et al., Alginate hydrogels as synthetic extracellular matrix materials, Biomaterials, (20):45-53 (doi:10.1016/S0142-9612(98)00107-0). Alginates are useful in the present invention, for example, to provide larger pore sized gels, which can be on the order of several hundred nanometers in size.

Alginate is a commonly used polymer for encapsulation of therapeutic agents (Goh C H et al., 2012. Alginates as a useful natural polymer for microencapsulation and therapeutic applications. Carbohydr. Polym., 88:1-12(2012) (doi:10.1016/j.carbpol.2011.11.012), and ever since the first successful microencapsulation of pancreatic islets was reported by Lim & Sun (Lim F et al., Microencapsulated islets as bioartificial endocrine pancreas, Science, 210:908-910 (1980) (doi:10.1126/science 6776628) it has become the most studied material for encapsulation of living cells (de Vos P et al., Alginate-based microcapsules for immunoisolation of pancreatic islets, Biomaterials, (27):5603-5617 (doi:10.1016/j.biomaterials.2006.07.010); Murua A et al., Cell microencapsulation technology: towards clinical application, J. Control. Release, 132:76-83 (2008) (doi:10.1016/j.jconrel.2008.08.010) When multi-valent cations (e.g. Ca²⁺) are added to a water-based alginate solution, they bind adjacent alginate chains forming ionic interchain bridges that cause a fast sol-gel transition compatible with the survival of the entrapped cells. It is generally assumed that cations bind preferably to the G blocks of the chains but relatively recent studies also suggest that the M block (in particular, the alternating MG block) has an active role in cross-linking the polymer chains (Donati I et al., New hypothesis on the role of alternating sequences in calcium-alginate gels, Biomacromolecules, 6:1031-1040 (2005) (doi:10.1021/bm049306e). In alginate, a naturally occurring biomaterial, the relative ratio between the G and M blocks is not constant and depends on the seaweed from which it is extracted. The G blocks provide rigidity to the polymeric structure and the mechanical properties of alginates are influenced by the ratio of G and M blocks, and as expected high G alginates result in the formation of stronger gels in compression (Mancini M et al., Mechanical properties of alginate gels: empirical characterization, J. Food Eng, 39:369-378 (1999) (doi:10.1016/S0260-8774(99)00022-9) and tension tests (Drury J L et al., The tensile properties of alginate hydrogels, Biomaterials, 25:3187-3199 (2004) (doi:10.1016/j.biomaterials.2003.10.002). Alginates can form polyelectrolyte complexes in the presence of polycations such as poly-L-lysine or chitosan. Poly-L-lysine has been widely used to coat the alginate beads as a way of controlling their molecular weight cut-off. A positively charged cation may be immunogenic and attract host inflammatory cells (Strand B et al., Poly-l-lysine induces fibrosis on alginate microcapsules via the induction of cytokines, Cell Transplant, 10:263-275 (2001) (doi:10.3727/000000001783986800); (Bhatia S R et al., Polyelectrolytes for cell encapsulation, Curr. Opin. Colloid Interface Sci., 10:45-51 (2005) (doi:10.1016/j.cocis.2005.05.004). For this reason, another external alginate coating is often added to the beads to form the so-called ‘alginate-polylysine-alginate’ (APA) system. However, developments in the characterization of APA capsules (Tam S K et al., Physicochemical model of alginate-poly-L-lysine microcapsules defined at the micrometric/nanometric scale using ATR-FTIR. XPS, and ToF-SIMS, Biomaterials, 26:6950-6961(2005) (doi:10.1016/j.biomaterials.2005.05.007), suggest that these capsules are not multi-layered; instead they consist of an inner calcium-alginate core covered by one single external layer of a poly-L-lysine and alginate blend. The binding strength of the initial poly-L-lysine layer depends on the relative ratio of the G and M blocks in the alginate core. Poly-L-lysine does not bind tightly to alginates with a high content of G blocks because, in contrast to M blocks, they do not allow complete interaction with the polycation. When these capsules are implanted or incubated they induce a stronger response than capsules without poly-L-lysine (Vos P D et al., Effect of the alginate composition on the biocompatibility of alginate-polylysine microcapsules, Biomaterials, 18:273-278 (1997) (doi:10.1016/S0142-9612(96)00135-4); Juste S et al., Effect of poly-L-lysine coating on macrophage activation by alginate-based microcapsules: assessment using a new in vitro method, J. Biomed. Mater. Res. A, 72:389-398 (2005) (doi:10.1002/jbm.a.30254).

Alginates can also be combined with other biopolymers to improve the biological response of the host. Such studies were recently performed using high-throughput methodologies for the evaluation of the in vitro (Salgado C L et al., Combinatorial cell-3D biomaterials cytocompatibility screening for tissue engineering using bioinspired supvrhydrophobic substrates, Integr. Biol., 4:318-327 (2012) (doi:10.1039/c2ib0070e), and in vivo (Oliveira M B et al., In press. In vivo high-content evaluation of three-dimensional scaffolds biocompatibility, Tissue Eng. Part C, Methods, (2012) (doi:10.1089/ten.TEC.2013.0738), response to different combinations of biomaterials. Furthermore, alginate does not provide cell adhesion motifs, but it can be conjugated with RGD peptides to improve cell adhesion (Yu J et al., The effect of injected RGD modified alginate on angiogenesis and left ventricular function in a chronic rat infarct model, Biomaterials, 30:751-756 (2009) (doi:10.1016/j.biomaterials.2008.09.059).

Alginate is characterized by a wide pore size distribution, which can range from about 5 nm to about 1μ, with the most open structure found in alginates with high G content (Smidsrod O et al, Alginate as immobilization matrix for cells, Trends Biotechnol. 8:71-78 (1990) (doi:10.1016/0167-7799(90)90139-O); Martinsen A et al., Alginate as immobilization material: I. Correlation between chemical and physical properties of alginate gel beads, Biotechnol. Bioeng, 33:79-89 (1989) (doi:10.1002/bit.260330111). The permeability of alginate is strongly influenced by the concentration and nature of the hardening ions; higher concentrations of ions create tighter structures (especially in the outer part of the gel in direct contact with the hardening bath) and as a consequence decrease the diffusion rate of large molecules outside the gel (Aslani P et al., Studies on diffusion in alginate gels. I. Effect of cross-linking with calcium or zinc ions on diffusion of acetaminophen, J. Control. Release, 42:75-82 (2006) (doi:10.1016/0168-3659(96)01369-7); Tanaka H et al., Diffusion characteristics of substrates in Ca-alginate gel beads, Biotechnol. Bioeng, 26:53-58 (1984) (doi:10.1002/bit.260260111). Instead, when the hardening bath consists of salts with low solubility in water (e.g. CaCo₃) the structure that is formed is more uniform and the hydrogel has higher mechanical stability (Kuo C K et al., Ionically crosslinked alginate hydrogels as scaffolds for tissue engineering: Part 1. Structure, gelation rate and mechanical properties, Biomaterials, 22:511-521(2001) (doi:10.1016/S0142-9612(00)00201-5) Furthermore, it should be noted that, as most of the proteins are negatively charged at pH 7, they do not easily diffuse into the gel while they diffuse out more quickly than expected (Smidsrod O et al., Alginate as immobilization matrix for cells, Trends Biotechnol. 8:71-78 (1990) (doi:10.1016/0167-7799(90)90139-0).

Agarose.

In some embodiments of the invention, agarose is a preferred gelation reagent. Agarose is a polysaccharide derived from the cell wall of a group of red algae (Rhodophyceae), including Gelidium and Gracilaria (Fu X T et al., Agarase: review of major sources, categories, purification method, enzyme characteristics and applications, Mar. Drugs, 8:200-218 (2010) (doi:10.3390/md8010200). The main structure of agarose consists of alternating units of β-D-galactopyranose and 3,6-anhydro-α-L-galactopyranose. Agarose extracted from different sources can have different chemical compositions; for example, sulfates can be found instead of the hydroxyl groups with a variable degree of substitution. Agarose is a responsive polymer and its aqueous solutions undergo a sol-gel transition upon cooling. Above the sol-gel temperature, agarose exhibits a random-coil conformation in solution, and upon cooling the structure changes to a double helix. Some of the helices then aggregate and the hydrogen bonds between structural water and galactose stabilize the structure (Lahaye M et al, Chemical structure and phYsico-chemical properties of agar, 137-148 (1991).

The gelling temperature depends on the concentration of the solution, the average molecular weight of the polymer and its structure. For this reason, there is a wide range of commercially available agarose, characterized by different gel strengths and sol-gel transition temperatures. Some of them can be used for cell encapsulation since their sol-gel transition occurs at around 37° C. The thermal sol-gel transition of agarose is reversible and presents a marked thermal hysteresis, which is a wide temperature difference between gelling and liquefaction (Indovina P L et al., Thermal hysteresis and reversibility of gel-sol transition in agarose-water systems, J. Chem. Phys., 70:2841 (1979) (doi:10.1063/1.437817).

The average pore size of agarose hydrogels and, as a consequence, the mass transport properties are influenced by the concentration of the polymer in solution and the settling temperature. An increase in concentration results in tightly packed helices that translate to a decrease in pore size (Pernodet N et al., Pore size of agarose gels by atomic force microscopy, Electrophoresis, 18:55-58 (1997) (doi:10.1002/elps.1150180111). For a Bio-Rad Certified low-melt agarose, Narayanan et al. (Narayanan J et al., Determination of agarose gel pore size: absorbance measurements vis a vis other techniques, J. Phys. Conf. Ser., 28:83-86 (2006) (doi:10.1088/1742-6596/28/1/017), measured an average pore size of 600 nm for a concentration of 1% w/v decreasing to 100 nm or less when the concentration was 3%. A decrease in settling temperature results in gel with smaller pores and higher elastic modulus (compression test). For example, Aymard et al. (Aymard P et al., Influence of thermal history on the structural and mechanical properties of agarose gels, Biopolymers, 59:131-144 (2001) (doi:10.1002/1097-0282(200109)59:3<131:AID-BIP1013>3.0 CO:2-8) showed a decrease in elastic modulus for a type I-A agarose (Sigma, 36° C. gelling temperature) from 78 kPa for samples cured at 5° C. to 53 kPa for samples cured at 35° C.

Agarose does not provide adhesion motifs to cells and does not allow interaction between adherent cells and the entrapping matrix (Tang S et al., Agarose/collagen composite scaffold as an anti-adhesive sheet, Biomed. Mater., 2:S129-S134 (2007) (doi:10.1088/1748-6041/2/3/S09) However, it can be supplemented with adhesion molecules of the extracellular matrix, such as fibronectin (Karoubi G et al., Single-cell hydrogel encapsulation for enhanced survival of human marrow stromal cells, Biomaterials, 30:5445-5455 (2009) (doi:10.1016/j.biomaterials.2009.06.035) or RGD soluble peptide (Guaccio A et al., Oxygen consumption of chondrocytes in agarose and collagen gels: a comparative analysis, Biomaterials, 29:1484-1493 (2008) (doi:10.1016/j.biomaterials.2007.12.020).

Agarose is not biodegradable—it can only be degraded by specific bacteria, not mammals. It can be degraded in vitro by agarases, which are classified according to their cleavage pattern into three types: α-agarase, β-agarase and β-porphyranase (Chi W-J et al., Agar degradation by microorganisms and agar-degrading enzymes, Appl. Microbiol. Biotechnol., 94:917-930 (2012) (doi:10.1007/s00253-012-4023-2); Zhang L-M et al., Synthesis and characterization of a degradable composite agarose/HA_hydrogel, Carbohydr. Polym., 88:1445-1452 (doi:10.1016/i.carbpol.2012.02.050); Emans P J et al., Autologous engineering of cartilage. Proc. Natl Acad. Sci. USA, 107:3418-3423 (2010) (doi:10.1073/pnas.0907774107).

Agarose is a preferred embodiment wherein the material captured in a gel-bead of the invention is subject to genomic sequencing.

pAm (polyacrylamide (poly(2-propenamide) or poly(1-carbamoylethylene

In further embodiments of the invention, pAm is the preferred gelation reagent. Polyacrylamide (IUPAC poly(2-propenamide) or poly(1-carbamoylethylene)) is a polymer (—CH₂CHCONH₂—) formed from acrylamide. Polyacrylamide may be admixed with another compound to form a composite. In the present invention, polyacrylamide is useful where smaller pore gets are desired, for example, in the range of about 1 nm to about 10 nm. In one embodiment of the invention, about 3% to about 20%, monomer is employed with about 0.1% to about 5% of a selected cross-linker.

Polyalkylene Glycol.

In some embodiments, a polyalkylene, such as “PEG,” is a preferred gelation reagent. Polyalkylene glycol polymers may be used in the present invention or in combination with a copolymer described above. Polyalkylene glycol polymers include, but are not limited, to straight or branched polyalkylene glycol polymers such as polyethylene glycol, polypropylene glycol, and polybutylene glycol, and further includes the monoalkylether of the polyalkylene glycol. The polyalkylene glycol polymer may be a lower alkyl polyalkylene glycol moiety such as a polyethylene glycol moiety (PEG), a polypropylene glycol moiety, or a polybutylene glycol moiety. PEG has the formula —HO(CH₂CH₂O)ₙH, where n can range from about 1-100, 5-30, or 1-4000. The PEG moiety can be linear or branched. PEG may be attached to groups such as hydroxyl, alkyl, aryl, acyl, or ester. For example, PEG may be an alkoxy PEG, such as methoxy-PEG (or mPEG), where one terminus is a relatively inert alkoxy group, while the other terminus is a hydroxyl group. Further polyalkylene glycol polymers include but are not limited to poly(ethylene glycol), poly(propylene glycol), and its copolymers, poly(ethylene glycol) copolymers with other synthetics such as poly(hydroxy acids), poly(vinyl alcohol), poly(vinyl pyrrolidone), and mixture thereof. In the present invention, PEG is useful where smaller pore gets are desired, for example, in the range of about 1 nm to about 10 nm. The molecular weight of PEG monomers and type of linking chemistry, for example, end-end; or ends-middle of a chain. In one preferred embodiment, an end-end relationship is preferred.

Cross-Linking Agents.

In the present invention, the rigidity, strength and pore size are affected by the amount of cross-linking. The materials described herein, including polymers, may be cross-linked using any suitable cross-linking agent as would be known to persons skilled in the art, for example, 1,4 butanediol diacrylate. Exemplary cross-linking agents may be any terminally ethylenically unsaturated compound having more than one unsaturated group (i.e., a multiplicity of unsaturated groups.) See, for example, U.S. Pat. No. 5,741,923. Other exemplary cross-linking agents include, but are not limited to: ethylene glycol diacrylate or dimethacrylate, diethylene glycol diacrylate or dimethacrylate, triethylene glycol diacrylate or dimethacrylate, tetraethylene glycol diacrylate or dimethacrylate, polyethylene glycol diacrylate or dimethacrylate, trimethylolpropane triacrylate or trimethacrylate, bisphenol A diacrylate or dimethacrylate, ethoxylated bisphenol A diacrylate or dimethacrylate, pentaerythritol tri- and tetra-acrylate or methacrylate, tetramethylene diacrylate or dimethacrylate, methylene bisacrylamide or methacrylamide, dimethylene bisacrylamide or methacrylamide, N,N′-dihydroxyethylene bisacrylamide or methacrylamide, hexamethylene bisacrylamide or methacrylamide, decamethylene bisacrylamide or methacrylamide, divinyl benzene, vinyl methacrylate, and allyl methacrylate. Additional exemplary cross-linking agents include 1,3-bis(4-methacryloyl oxyalkyl)tetra disiloxane and similar poly(organo-siloxane) monomers. See, for example, U.S. Pat. No. 4,153,641. Another group of exemplary cross-linking agents are the resonance-free di(alkylene tertiary amine) cyclic compounds (e.g., N,N′-divinyl ethylene urea). See, for example, U.S. Pat. No. 4,436,887. Further exemplary cross-linking agents include di- or polyvinyl ethers of di- or polyvalent alcohols such as ethylene glycol divinyl ether.

In some embodiments of the invention, droplets are rapidly gelled on a microsurface, for example, a chip, through a variety of techniques. These techniques include, but are not limited to the use of temperature, chemical stimulation or light stimulation. Illustrative polymers described herein include temperature-, pH-, ion- and/or light-sensitive polymers. Hoffman, A. S., “Intelligent Polymers in Medicine and Biotechnology,” Artif. Organs. 19:458-467 (1995); Chen, G. H. and A. S. Hoffman, “A New Temperature- and Ph-Responsive Copolymer for Possible Use in Protein Conjugation”, Macromol. Chem. Phys. 196:1251-1259 (1995); Irie, M. and D. Kungwatchakun, “Photoresponsive Polymers. Mechanochemistry of Polyacrylamide Gels Having Triphenylmethane Leuco Derivatives”, Maokromol. Chem., Rapid Commun 5:829-832 (1985); and Irie, M., “Light-induced Reversible Conformational Changes of Polymers in Solution and Gel Phase”, ACS Poym. Preprinis, 27(2):342-343 (1986); which are incorporated by reference herein.

Temperature-Sensitive Polymers.

Illustrative embodiments of the many different types of temperature-sensitive polymers useful in the present invention, which may be conjugated to interactive molecules are polymers and copolymers of N-isopropyl acrylamide (NIPAAm). PolyNIPAAm is a thermally sensitive polymer that precipitates out of water at 32° C., which is its lower critical solution temperature (LCST), or cloud point (Heskins and Guillet, J. Macromol. Sci.-Chem. A2:1441-1455 (1968)). When polyNIPAAm is copolymerized with more hydrophilic comonomers such as acrylamide, the LCST is raised. The opposite occurs when it is copolymerized with more hydrophobic comonomers, such as N-t-butyl acrylamide. Copolymers of NIPAAm with more hydrophilic monomers, such as AAm, have a higher LCST, and a broader temperature range of precipitation, while copolymers with more hydrophobic monomers, such as N-t-butyl acrylamide, have a lower LCST and usually are more likely to retain the sharp transition characteristic of PNIPAAm (Taylor and Cerankowski, J. Polymer Sci. 13:2551-2570 (1975); Priest et al., ACS Symposium Series 350:255-264 (1987); and Heskins and Guillet, J. Macromol. Sci.-Chem. A2:1441-1455 (1968), the disclosures of which are incorporated herein). Copolymers can be produced having higher or lower LCSTs and a broader temperature range of precipitation.

Light-Sensitive Polymers.

Light-responsive polymers useful in the present invention, typically contain chromophoric groups pendant to or along the main chain of the polymer and, when exposed to an appropriate wavelength of light, can be isomerized from the trans to the cis form, which is dipolar and more hydrophilic and can cause reversible polymer conformational changes. Other light sensitive compounds can also be converted by light stimulation from a relatively non-polar hydrophobic, non-ionized state to a hydrophilic, ionic state. In the case of pendant light-sensitive group polymers, the light-sensitive dye, such as aromatic azo compounds or stilbene derivatives, may be conjugated to a reactive monomer (an exception is a dye such as chlorophyllin, which already has a vinyl group) and then homopolymerized or copolymerized with other conventional monomers, or copolymerized with temperature-sensitive or pH-sensitive monomers using the chain transfer polymerization as described above. The light sensitive group may also be conjugated to one end of a different (e.g., temperature) responsive polymer. Although both pendant and main chain light sensitive polymers may be synthesized and are useful compositions for the methods and applications described herein, the preferred light-sensitive polymers and copolymers thereof are typically synthesized from vinyl monomers that contain light-sensitive pendant groups. Copolymers of these types of monomers are prepared with “normal” water-soluble comonomers such as actylamide, and also with temperature- or pH-sensitive comonomers such as NIPAAm or AAc.

Specific Ion-Sensitive Polymers.

Polysaccharides useful in the present invention, such as carrageenan, that change their conformation, for example, from a random to an ordered conformation, as a function of exposure to specific ions, such as K⁺ or Ca⁺⁺, can also be used as the stimulus-responsive polymers. In another example, a solution of sodium alginate may be gelled by exposure to Ca⁺⁺. Other specific ion-sensitive polymers include polymers with pendant ion chelating groups, such as histidine or EDTA.

Dual- or Multi-Sensitivity Polymers.

If a light-sensitive polymer is employed in the present invention, and is also thermally-sensitive, the UV- or visible light-stimulated conversion of a chromophore conjugated along the backbone to a more hydrophobic or hydrophilic conformation can also stimulate the dissolution or precipitation of the copolymer, depending on the polymer composition and the temperature. If the dye absorbs the light and converts it to thermal energies rather than stimulating isomerization, then the localized heating can also stimulate a phase change in a temperature-sensitive polymer such as PNIPAAm, when the system temperature is near the phase separation temperature. The ability to incorporate multiple sensitivities, such as temperature and light sensitivity, or temperature and pH sensitivity, along one backbone by vinyl monomer copolymerization lends great versatility to the synthesis and properties of the responsive polymer-protein conjugates. For example, dyes can be used which bind to protein recognition sites, and light-induced isomerization can cause loosening or detachment of the dye from the binding pocket (Bieth et al., Proc. Natl. Acad. Sci. USA 64:1103-1106 (1969)). This can be used for manipulating affinity processes by conjugating the dye to the free end of a temperature responsive polymer, such as ethylene oxide-propylene oxide (EO-PO) random copolymers available from Carbide. These polymers, —(CH₂CH₂O)ₓ—(CH₂CHCH₃O)_{y}—, have two reactive end groups. The phase separation point can be varied over a wide range, depending on the EO/PO ratio, and one end may be derivatized with the ligand dye and the other end with an —SH reactive group, such as vinyl sulfone (VS).

Stabilizing Membrane

In some embodiments of the invention, a stabilizing membrane is employed to protect the formed droplets. Stabilizing membranes, such as “nylon,” can formed by the introduction of selected monomer reagents introduced into the core solution and oil droplets and subsequently formed at the interphase between the two. Advantageously, these formed membranes yield a stabilized droplet until a gel is formed. After formation of the gel, the membrane can be removed, for example, subsequently broken by a later reaction. Such reagents, include, for example disulfides provided with the monomers, which be broken in a reducing environment. Additionally, groups that are broken by a protease, for example, “linkers” used to deliver drugs with short peptides for cleaving the drug off of an antibody or other delivery device. An additional process includes combining the monomers with nucleotides, which are subsequently broken by a nuclease.

Monomers useful for the formation of a stabilizing nylon (polyamide) membrane include, for example, ε-Caprolactam, hexamethylenediamine and adipic acid, Hexamethylenediamine and azelaic acid, Hexamethylenediamine with sebacic acid, hexamethylenediamine with dodecanedioic acid, 11-amino undecanoic acid and laurolactam. However, it will be appreciated that any known monomer suitable for producing a polyamide when polymerized may be used in the present invention.

In some embodiments of the invention, a linker is employed. As used herein, the term “linker,” means an organic moiety that connects two parts of a compound. Linkers are typically characterized as having a direct bond or an atom such as oxygen or sulfur, a unit such as NH, C(O), C(O)NH, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylal kyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, NH, C(O). The terms linker and spacer are used interchangeably herein. The linker can contain any combinations of the above. Accordingly, in some embodiments, the linker can comprise hydrocarbons, amino acids, peptides, polyethylene glycol of various lengths, cyclodextrins, and derivatives and any combinations thereof.

In some embodiments, the linker is a branched linker. A branched linker can be used to connect two or more (e.g., 2, 3, 4, 5, 6, 7, 8, 9, 10 or more) molecules of interest (which can be same or different) to one affinity ligand; two or more (e.g., 2, 3, 4, 5, 6, 7, 8, 9, 10 or more) affinity ligands (which can be same or different) to one molecule of interest; or two or more (e.g., 2, 3, 4, 5, 6, 7, 8, 9, or more) molecules of interest (which can be same or different) to two or more (e.g., 2, 3, 4, 5, 6, 7, 8, 9, 10 or more) affinity ligands (which can be same or different).

In some embodiments, the linker comprises at least one cleavable linking group. A cleavable linking group is one which is sufficiently stable outside the cell, but which upon entry into a target cell is cleaved to release the two parts the linker is holding together. Cleavable linking groups are susceptible to cleavage agents, for example, pH, redox potential or the presence of degradative molecules. Examples of such degradative agents include: redox agents which are selected for particular substrates or which have no substrate specificity, including, e.g., oxidative or reductive enzymes or reductive agents such as mercaptans, which can degrade a redox cleavable linking group by reduction; esterases; amidases; endosomes or agents that can create an acidic environment, e.g., those that result in a pH of five or lower; enzymes that can hydrolyze or degrade an acid cleavable linking group by acting as a general acid, peptidases (which can be substrate specific) and proteases, and phosphatases. The cleavable linking group can comprise esters, peptides, carbamates, acid-labile, reduction-labile, oxidation-labile, disulfides, and modifications thereof. A linker can include a cleavable linking group that is cleavable by a particular enzyme.

### Core-Shell Beads

Core-shell beads of the present invention, are prepared in a similar manner as detailed herein, however, the microfluidic device is typically further characterized by having a first laminar cross flow, which contains a gel, for example, a monomer solution, which subsequently forms a transient solution with core fluid on the inside and the gel solution on the outside creating fluid columns. As the fluid column encounters the oil from a second laminar cross flow, droplets characterized with an inner aqueous core and an outer gel phase are formed. The inner aqueous core, which is a liquid having a gel shell, permits the introduction of a “scaffold” or “scaffold molecule” within the gel, which is able to capture and retain desired molecules. The cells and molecules attached to the scaffold are trapped in the gels phase or alternatively, in the aqueous core of the core-shell structures.

### Molecular Retention Employing a Scaffold

The term “scaffold” or “scaffold molecule,” as used herein, indicates a molecular structure of a capture agent that serves to assemble an affinity agent (e.g., MHC) to an encoding polynucleotide (e.g., ssDNA tags). This structure can be a magnetic particle such as a magnetic bead that is conjugated to an affinity agent. This structure can be derived from proteins (such as Streptavidin, biotin or SA), other biopolymers (such as polynucleotides, like RNA and DNA, peptide nucleic acid, etc.), or other polymers which can bind to the affinity agent and the encoding polynucleotide in distinct and separate portions of the polymer. Capture agents of the invention can also include antibodies and complementary ligands. In the present invention, a scaffold or scaffold molecule is prepared in manner, wherein the scaffold is larger than the pore size of a gel matrix.

The wording “polynucleotide-encoded capture agent” refers to a polynucleotide encoded molecular construct that specifically binds to a target. In particular, a polynucleotide-encoded capture agent typically comprises a binding component that specifically binds to, and is thereby defined as complementary to, the target, a structural component that supports the binding component and an encoding polynucleotide attached to the structural component that encodes the molecular structure.

In a “modular polynucleotide-encoded capture agent” the binding component, the structural component and the encoding component of the polynucleotide encoded capture agent are formed by standardized molecular units that can be coupled or decoupled to each other in a controlled fashion. In particular, in the modular polynucleotide-encoded capture agents herein described, the binding component is formed by at least one binding molecule, that is configured to specifically bind to, and be thereby defined as complementary to, a target; the encoding component is formed by an encoding polynucleotide configured to specifically bind, and be thereby defined as complementary to, a substrate polynucleotide attached to a substrate, and the structural component is formed by a scaffold molecule attaching the at least one binding molecule and the encoding polynucleotide. In particular, in the modular polynucleotide-encoded capture agents, the at least one binding molecule specifically binding to a target, the scaffold molecule and an encoding polynucleotide, are attached or to be attached one to the other.

The term “attach” or “attached” as used herein, refers to connecting or uniting by a bond, link, force or tie in order to keep two or more components together, which encompasses either direct or indirect attachment such as, embodiments where a first molecule is directly bound to a second molecule or material, and embodiments wherein one or more intermediate molecules are disposed between the first molecule and the second molecule or material. Molecules include but are not limited to polynucleotides, polypeptides, and in particular proteins and antibodies, polysaccharides, aptamers and small molecules.

In modular polynucleotide encoded capture agents here described, the scaffold molecule is configured to bind the at least one binding molecule and an encoding polynucleotide, with scaffold binding domains. The term “domain” as used herein with indicates a region that is marked by a distinctive structural and functional feature. In particular, a scaffold binding domain is a region of the scaffold that is configured for binding with another molecule. Accordingly, a scaffold binding domain in the sense of the present disclosure includes a functional group for binding the another molecule and a scaffold binding region on the scaffold that is occupied by the another molecule bound to the scaffold. Once the functional group has been identified, the relevant scaffold binding region can be determined with techniques suitable to identify the size and in particular the largest diameter of the another molecule of choice to be attached. The average largest diameter for a protein according to the present disclosure in several embodiments is between about 10 Å and about 50 Å depending on the protein of choice, between about 3 Å and about 10 Å for a small molecule, and is between about 10 Å and about 20 Å for a polynucleotide. Techniques suitable to identify dimensions of a molecule include, but are not limited, to X-ray crystallography for molecules that can be crystallized, and techniques to determine persistence length for molecules such as polymers that cannot be crystallized. Those techniques for detecting a molecule dimensions are identifiable by a skilled person upon reading of the present disclosure.

In some embodiments, the scaffold can be configured to enable or ease attachment of multiple copies of single-stranded encoding polynucleotide (e.g. DNA oligomers) in multiple second scaffold binding domains. In those embodiments, the second scaffold binding domain can be selected to allow hybridization with an encoding polynucleotide to be used to spatially direct the scaffold to particular spots on a surface that are coated with the substrate polynucleotides.

A scaffold, thus configured, can be useful, in embodiments where the modular polynucleotide-encoded capture agents is used for the spatially selective sorting of specific cell types. For example, multiple scaffolds, each containing a different set of affinity agents, and uniquely labeled with bindingly distinguishable ssDNA oligomers, can be harnessed in parallel to spatially separate a mixture of many cell types into its individual components as it will be apparent to a skilled person in view of the present disclosure. For example, in some embodiments, it is feasible to use modular capture agents with biotinylated-antibodies along with p/MHC proteins as the affinity reagents, where each is encoded to bindingly distinguishable ssDNA oligomers. The antibodies can be used to sort cells according to cell surface markers like CD4, CD8, CD3, etc., while the p/MHC proteins will sort cells according to antigen-specificity as determined by the TCRs.

In some embodiments, a desired configuration of a scaffold and, in particular, a scaffold protein, can be achieved through modification of candidate scaffolds that are modified with techniques known to the skilled person such as traditional cloning techniques or other techniques identifiable by a skilled person.

In some embodiments, the scaffold can be optimized for a specific capture agent. In particular, in a specific capture agent an optimized scaffold has well defined scaffold binding regions for independently coupling a binding molecule and an encoding-polynucleotide, so that upon binding the binding molecule and the encoding polynucleotide, possible interferences between the polynucleotide and the assembly of the binding molecule are minimized This is usually achieved for a capture agent having a desired binding affinity for the target and the substrate polynucleotide, by minimizing structural overlapping between the binding molecule(s) and the encoding polynucleotide attached to the scaffold while maintaining a desired binding affinity of the capture agent for the target and the substrate polynucleotide.

Accordingly, in several embodiments where the scaffold protein is streptavidin, binding molecules (e.g. MHC molecules) can be biotinylated, to enable the tetrameric assembly with the protein-ligand pair SA. In some embodiments, binding molecules can also be coupled to SA via covalent linkages (such as amide coupling), and therefore not necessarily through the biotin-SA interaction. The skilled person will be able to identify the most appropriate binding based on the experimental design of choice. In several embodiments of the present disclosure, SA is used as standard scaffold used to assemble p/MHC monomers into tetramers.

In embodiments where the scaffold is SA, a modified SA can be used as well as molecules derived therefrom (see in particular SA-phycobiliprotein (PE or APC) conjugates). In some embodiments, a scaffold can be used that is a recombinant mutant of SA for fluorescent p/MHC tetramer preparations. In some of those embodiments, SA variants can be used, such as for example a variant that incorporates a cysteine residue at the carboxy-terminus [Ref 25, 26, 27], in a site removed from the biotin binding pocket. In those embodiments, the conjugation of cysteine-reactive maleimide derivatives can be restricted to the C-terminus because cysteine residues are absent in native SA.

Functional groups for binding a binding molecule, that can be included in a first scaffold binding domain, depend on the chemical nature of the binding molecule and are identifiable by the skilled person upon reading of the present disclosure. For example, functional groups for binding a binding molecule include but are not limited to BirA Ligase (enzyme that attaches biotin group to predefined peptide sequences), other enzymes such as formylglycine-generating enzyme (site-specific introduction of aldehyde groups into recombinant proteins.

Functional groups for binding a polynucleotide, that can be included in a second scaffold binding domain, are also identifiable by the skilled person upon reading of the present disclosure. Exemplary functional groups presented on the scaffold for binding a polynucleotide include functional groups such as sulfulhydryl (e.g. in a cysteine residue), primary amines and other functional groups that attach derivatized DNA via conventional conjugation strategies, that would be identifiable by the skilled reader.

Functional groups can either be endogenous groups on the scaffold (e.g. native lysine residues on a scaffold protein), or introduced by methods such as gene cloning (e.g. proteins), synthetic techniques (polymers, small molecules), and other methods. The number of copies of polynucleotides or binding molecules that can attach to the scaffold will be directly proportional to the number of functional groups available on the scaffold.

In some embodiments, in addition to containing distinct scaffold binding domains to accommodate the affinity agent and encoding DNA, the scaffold is also selected to be compatible with the environment of the target of interest (e.g. it should be soluble in aqueous solutions if the target is cell surface markers).

In some additional embodiments, the scaffold consists of a macromolecular scaffold that is customized, via multi-ligand interactions, for the high affinity binding to specific cell types, and then for the spatially directed, multiplexed sorting of those different cell types.

In other embodiments, the scaffold is provided by a non-naturally occurring molecule that is expressed with modular design characteristics. In those embodiments, the protein scaffold is designed so that multiple and controlled numbers of copies of specific binding molecules and encoding polynucleotides may be attached to the scaffold at specific scaffold polynucleotide binding domains.

Collecting and/or Incubating Gel-Beads

Gel-beads of the invention can optionally be collected, incubated and/or stored and processed by a variety of methods and techniques. Such methods include, but are not limited to: destabilizing/washing the emulsion with oil and/or solvents; washing the emulsion with a variety of aqueous buffers; washing the gel-beads with solvents; washing the gel beads with aqueous buffers. Collection includes, for example, moving the cell containing gel beads into another vessel, thus physically separating the gel beads containing a cell or cellular material from those gel beads that lack cells or cellular material.

### Applications Using Gel-Beads of the Invention

Gel-beads and Core-shell beads of the invention can be utilized in a variety of assays. Such assays include, but are not limited to: cell culture, such as, cell growth assays, cell differentiation assays and transfection assays. The term “assay” or “assaying” as used herein, refers to an analysis to determine, for example, the presence, absence, quantity, extent, kinetics, dynamics, or type of a target, such as a cell's optical or bioimpedance response upon stimulation with exogenous stimuli (e.g., therapeutic agent). Multiple molecular biology uses, such as, PCR, RT, digestion and ligation are also envisioned in the present invention. Cell biology applications include, for example, cellular staining. Mechanical applications include, for example: Flow cytometry/FACS; loading into nano-well arrays; and loading into microfluidic droplets. PCR applications can be performed on gel beads by placing the beads in oil.

Other applications include cell proliferation assays, wherein testing the effects of pharmacological agents or growth factors, assessing cytotoxicity or investigating circumstances of cell activation. In a cell proliferation assay, cell numbers are measured, or measuring the change in the proportion of cells, that is dividing. There are four main types of cell proliferation assays, and they differ according to what is actually measured: DNA synthesis, metabolic activity, antigens associated with cell proliferation and ATP concentration.

A reliable and accurate assay type is the measurement of DNA synthesized in the presence of a label. Traditional cell proliferation assays involve incubating cells for a few hours to overnight with 3H-thymidine. Proliferating cells incorporate the radioactive label into their nascent DNA, which can be washed, adhered to filters and then measured using a scintillation counter.

Another measure of cell proliferation is the metabolic activity of a population of cells. Tetrazolium salts or Alamar Blue, are compounds that become reduced in the environment of metabolically active cells, forming a formazan dye that subsequently changes the color of the media. This is caused by increased activity of the enzyme lactate dehydrogenase during proliferation. The absorption of the media-containing dye solution can be read using a spectrophotometer or microplate reader in low- or high-throughput configurations.

Another method to measure cell proliferation is to detect an antigen present in proliferating cells, but not nonproliferating cells, using a monoclonal antibody to the antigen. For example, in human cells, the antibody Ki-67 recognizes the protein of the same name, expressed during the S, G2 and M phases of the cell cycle but not during the G0 and G1 (nonproliferative) phases.

Another type of cell proliferation assay takes advantage of the tight regulation of intracellular ATP within cells. Dying or dead cells contain little to no ATP, so there is a tight linear relationship between cell number and the concentration of ATP measured in a cell lysate or extract. The bioluminescence-based detection of ATP, using the enzyme luciferase and its substrate luciferin, provides a very sensitive readout. In the presence of ATP, luciferase produces light (proportional to the ATP concentration) that can be detected by a luminometer or any microplate reader capable of reading luminescent signals. This approach is also well suited to high-throughput cell proliferation assays and screening.

Another method to measure cell proliferation is to detect replication of cells inside a gel-bead or droplet by measurement with a cytometer and a cell specific stain. In this manner it is possible to count the number of cells present in a droplet or gel-bead and sort individual gel-beads or droplets on the basis of count or growth characteristics of the “colony” of cells inside the droplet or gel-bead.

### Sequencing of Immune Binding Proteins

In some embodiments, the amplified nucleic acids are used in a sequencing reaction and the OE region can be flanked by one or more barcode regions (BC1 and BC2) (FIG. 1b). In some embodiments, the nucleic acids encoding the multiple chains of the immune binding protein are sequenced to identify the chains which form the immune binding protein (e.g., the heavy and light chains of an antibody).

Sequencing tools, methods, apparati, and reagents are well known to the person of ordinary skill in the art and include, for example, single-molecule real-time sequencing (Pacific Biosciences), ion semiconductor (Ion Torrent sequencing of Thermo Fisher), pyrosequencing (454 Life Sciences of Roche Diagnostics), sequencing by synthesis (Illumina), sequencing by ligation (SOLiD sequencing, Thermo Fisher), DNA nanoball sequencing (Complete Genomics), heliscope sequencing (Helicos Biosciences), and chain termination (Sanger sequencing). Sequencing machines and reagents are commercially available for all of these techniques, including for example, from Pacific Biosciences, Thermo Fisher, Roche Diagnostics, Illumina, Complete Genomics, and Helicos Biosciences.

In some embodiments, the resulting sequences are characterized for putative lineage information based on sequence alignment. In some embodiments, the sequence information is analyzed for similarity scores between sequences using bioinformatics tools (e.g. BLAST), and then optionally grouped into a phylogeny tree based on this information.

In some embodiments, sequences are compared using techniques well known to the person of ordinary skill in the art, including, for example, the local homology algorithm of Smith and Waterman, Adv Appl Math. 2:482, 1981; the homology alignment algorithm of Needleman and Wunsch, J Mol Biol. 48:443, 1970; the search for similarity method of Pearson and Lipman, Proc Natl Acad Sci. USA 85:2444, 1988; computerized implementations of these algorithms (GAP, BESTFIT, FASTA, and TFASTA in the GCG Wisconsin Software Package), or visual inspection (see generally, Current Protocols in Molecular Biology, F. M. Ausubel et al., eds., Greene Publishing Associates, Inc. and John Wiley & Sons, Inc., (1995 Supplement). Examples of algorithms that are suitable for comparing percent sequence identity and sequence similarity are the BLAST and BLAST 2.0 algorithms, which are described in Altschul et al., J. Mol. Biol. 215:403-410, 1990; and Altschul et al., Nucleic Acids Res. 25(17):3389-3402, 1977; respectively. Software for performing BLAST analyses is publicly available through the National Center for Biotechnology Information website. BLAST for nucleotide sequences can use the BLASTN program with default parameters, e.g., a wordlength (W) of 11, an expectation (E) of 10, M=5, N=−4, and a comparison of both strands. BLAST for amino acid sequences can use the BLASTP program with default parameters, e.g., a wordlength (W) of 3, an expectation (E) of 10, and the BLOSUM62 scoring matrix (see Henikoff and Henikoff, Proc Natl Acad Sci. USA 89:10915, 1989). Exemplary determination of sequence alignment and % sequence identity can also employ the BESTFIT or GAP programs in the GCG Wisconsin Software package (Accelrys, Madison Wis.), using default parameters provided.

### Repertoires of Immune Binding Proteins

The invention relates to nucleic acids encoding immune binding proteins that preserve the in vivo multimeric associations of the immune polypeptide chains making up the immune binding protein (e.g., antibodies, T-lymphocyte receptors or innate immunity receptors). In some embodiments, immune binding protein libraries of the invention are enriched for nucleic acids encoding multimers that are functional polypeptides representing the multimeric complexes found in the repertoire from which the immune binding protein library was obtained.

In some embodiments, the nucleic acids represent the antibody repertoire of a subject who has become immune to an infectious disease, cancer, or other immunogenic challenge. In some embodiments, the nucleic acids represent the antibody repertoire of a subject who has had an immune reaction to an infectious disease, cancer, or other immunogenic challenge. In some embodiments, the antibody repertoire is from a subject that is naïve for the target antigen. In some embodiments, the antibody repertoire represents the germ line repertoire of a subject or species. In some embodiments, the nucleic acids encoding the heavy and light chains of the antibody are combined in appropriate combinatorial fashion to generate a repertoire of antigen binding domains from the heavy and light chains.

In some embodiments, the repertoire represents the T-cell receptor repertoire of a subject who has become immune to an infectious disease, cancer, or other immunogenic challenge. In some embodiments, the nucleic acids represent the T-cell receptor repertoire of a subject who has had an immune reaction to an infectious disease, cancer, or other immunogenic challenge. In some embodiments, the T-cell receptor repertoire is from a subject that is naïve for the target antigen. In some embodiments, the T-cell receptor repertoire represents the germ line repertoire of a subject or species. In some embodiments, the nucleic acids encoding the alpha, beta, gamma and zeta chains of the T-cell receptor are combined in appropriate combinatorial fashion to generate a repertoire of antigen binding domains from the T-cell receptor chains.

In some embodiments, the nucleic acids represent the innate immunity receptor repertoire of a subject who has become immune to an infectious disease, cancer, or other immunogenic challenge. In some embodiments, the nucleic acids represent the innate immunity receptor repertoire of a subject who has had an immune reaction to an infectious disease, cancer, or other immunogenic challenge. In some embodiments, the innate immunity receptor repertoire is from a subject that is naïve for the target antigen. In some embodiments, the innate immunity receptor repertoire represents the germ line repertoire of a subject or species.

In some embodiments, the nucleic acids encoding the polypeptide chains for immune binding proteins are derived from individuals whom have mounted an immune response relevant to, for example, an infectious disease, a cancer, an autoimmune disease, an allergy, or a neurodegenerative disease. In some embodiments, the infectious disease is caused by an influenza virus. In some embodiments, the infectious disease is caused by a virus such as, for example, HIV, Ebola, Zika, HSV, RSV, or CMV.

Homologs of immune binding polypeptides of the invention are intended to be within the scope of the present invention. As used herein, the term “homologs” includes analogs and paralogs. The term “analogs” refers to two polynucleotides or polypeptides that have the same or similar function, but that have evolved separately in unrelated host organisms. The term “paralogs” refers to two polynucleotides or polypeptides that are related by duplication within a genome. Paralogs usually have different functions, but these functions may be related. Analogs and paralogs of an immune binding protein can differ from the immune binding protein by post-translational modifications, by amino acid sequence differences, or by both. In particular, homologs of the invention will generally exhibit at least 80-85%, 85-90%, 90-95%, or 95%, 96%, 97%, 98%, 99% sequence identity, with all or part of the immune binding protein or its polynucleotide sequences, and will exhibit a similar function. Variants include allelic variants. The term “allelic variant” refers to a polynucleotide or a polypeptide containing polymorphisms that lead to changes in the amino acid sequences of a protein and that exist within a natural population (e.g., a virus species or variety). Such natural allelic variations can typically result in 1-5% variance in a polynucleotide or a polypeptide. Allelic variants can be identified by sequencing the nucleic acid sequence of interest in a number of different species, which can be readily carried out by using hybridization probes to identify the same genetic locus in those species. Any and all such nucleic acid variations and resulting amino acid polymorphisms or variations that are the result of natural allelic variation and that do not alter the functional activity of the immune binding protein, are intended to be within the scope of the invention.

As used herein, the term “derivative” or “variant” refers to an immune binding protein, or a nucleic acid encoding an immune binding protein, that has one or more conservative amino acid variations or other minor modifications such that the corresponding polypeptide has substantially equivalent function when compared to the wild type polypeptide. These variants or derivatives include polypeptides having minor modifications of the immune binding protein primary amino acid sequences that may result in peptides which have substantially equivalent activity as compared to the unmodified counterpart polypeptide. Such modifications may be deliberate, as by site-directed mutagenesis, or may be spontaneous. The term “variant” further contemplates deletions, additions and substitutions to the sequence, so long as the polypeptide functions as an immune binding protein. The term “variant” also includes modification of a polypeptide where the native signal peptide is replaced with a heterologous signal peptide to facilitate the expression or secretion of the polypeptide from a host species.

The immune binding proteins of the invention also may include amino acid sequences for introducing a glycosylation site or other site for modification or derivatization of the polypeptide. In an embodiment, the polypeptides of the invention describe above may include the amino acid sequence N-X-S or N-X-T that can act as a glycosylation site. During glycosylation, an oligosaccharide chain is attached to asparagine (N) occurring in the tripeptide sequence N-X-S or N-X-T, where X can be any amino acid except Pro. This sequence is called a glycosylation sequon. This glycosylation site may be placed at the N-terminus, C-terminus, or within the internal sequence of the protein sequence used for the polypeptide of the invention.

### Display Libraries of the Immune Binding Proteins

In some embodiments, the nucleic acids encoding immune binding proteins of the invention are engineered into vectors for displaying the immune binding protein on the surface of a cell or a viral particle. In some embodiments, repertoires of immune binding proteins (e.g., antibodies, T-cell receptors, or innate immunity receptors) are displayed on filamentous bacteriophage (e.g., McCafferty et al., 1990, Nature 348:552-554, which is incorporated by reference in its entirety for all purposes), yeast cells (e.g., Boder and Wittrup, 1997, Nat Biotechnol 15:553-557, which is incorporated by reference in its entirety for all purposes), and ribosomes (e.g., Hanes and Pluckthun, 1997, Proc Natl Acad Sci USA 94:4937-4942, which is incorporated by reference in its entirety for all purposes). Other embodiments of phage display are disclosed in, for example, U.S. Pat. Nos. 5,750,373, 5,733,743, 5,837,242, 5,969,108, 6,172,197, 5,580,717, and 5,658,727, all of which are incorporated by reference in their entirety for all purposes.

In some embodiments, phage display libraries are used to make human antibodies, T-cell receptors (or parts thereof), or innate immunity receptors (or parts thereof) from immunized humans, non-immunized humans, germ line sequences, or naive repertories (Barbas & Burton, Trends Biotech (1996), 14:230; Griffiths et al., EMBO J. (1994), 13:3245; Vaughan et al., Nat. Biotech. (1996), 14:309; Winter EP 0368 684 B1, all of which are incorporated by reference in their entirety for all purposes). In some embodiments, naive, or nonimmune, antigen binding libraries are generated using a variety of lymphoidal tissues. Some of these libraries are commercially available, such as those developed by Cambridge Antibody Technology and Morphosys (Vaughan et al. (1996) Nature Biotech 14:309; Knappik et al. (1999) J. Mol. Biol. 296:57, all of which are incorporated by reference in their entirety for all purposes).

In some embodiments, Fab molecules can be displayed on phage if one of the chains (heavy or light) is fused to g3 capsid protein and the complementary chain exported to the periplasm as a soluble molecule. The two chains can be encoded on the same or on different replicons; the two antibody chains in each Fab molecule assemble post-translationally and the dimer is incorporated into the phage particle via linkage to one of the chains of g3p (see, e.g., U.S. Pat. No. 5,733,743, which is incorporated by reference in its entirety for all purposes). Alternatively, a scFv can be fused to a g3 capsid protein for display on the phage particle.

In some embodiments, nucleic acids encoding repertoires of immune binding proteins are engineered into vectors for display on bacterial, yeast, or mammalian cells. In some embodiments, bacterial, yeast or mammalian cells displaying immune binding proteins of the invention are contacted with a fluorescently labeled antigen, cells that bind the fluorescently labeled antigen will be fluorescent, and can then be isolated using fluorescence-activated cell sorting. In some embodiments, panning approaches are used to associate immune binding proteins with antigens bound by the immune binding protein.

In some embodiments, a library of immune binding proteins is engineered into a phage display vector and transformed into cells to generate phage which display the immune binding protein of interest in a fusion with one of the phage coat proteins. The phage library can be contacted with (aka panned against) a surface (e.g. a microtiter plate) that is coated with test antigens of interest. The plate is then washed one or more times with buffer. Phage that contain antibody variants that bind to the antigen of interest will be retained, whereas those that do not bind to the antigen will be washed away. The resulting phage library can subsequently be transformed into other host cells for further screening or replication and/or characterized by sequencing.

In some embodiments, the heavy chain/light chain pair of an antibody can be inserted into a surface display vector and cells can be transformed with this vector to display the antibody on the surface. Separately, a set of one or more antigens can be linked to a set of identifying nucleic acid barcode sequences such that each different antigen is linked to a unique sequence. The linkage can be done chemically or alternatively by cloning a set of barcoded antigens into a suitable display vector and expressing the antigen on the surface of phage or cells. The antigen set, now linked to a nucleic acid identifier, can then be contacted with the cells which display antibody on the surface. After the incubation, the individual cells can be isolated via emulsion, single-cell sorting, or other means. The resulting isolate will consist of a single cell displaying a homogeneous antibody on its surface, bound to one or more of the barcoded antigens. The nucleic acids coding for the antibody heavy chain, light chain, and antigen barcode, can then be amplified together and sequenced. The resulting sequence information will yield antibody/antigen coupling information. For example, if one antibody binds exclusively to a single antigen, the resulting sequence information will yield a unique antibody/antigen sequence. If an antibody binds a plurality of antigens, it will yield a mixed population of antibody/antigen coupled sequences. Thus, the relative specificity of each antibody in the population with respect to a set of antigens can be determined. Moreover, the relative abundance of the different coupled species can be correlated to the relative affinity of an antibody to each of the antigens in a panel.

In some embodiments, the pair can be cloned into a chimeric antigen receptor. A chimeric antigen receptor construct consists of at least a binding region (typically an scFv) and an intracellular signaling region. It may additionally contain other components such as a transmembrane region, a spacer/linker region, multiple signaling regions, and/or protein targeting and translocation sequences. Chimeric antigen receptors are well known in the art as described in, for example, U.S. patent application US20140242701, and U.S. Pat. Nos. 5,359,046, 5,686,281 and 6,103,521, which are incorporated by reference in their entirety for all purposes. The construct is placed into cells and the receptor is expressed, typically though not necessarily on the surface of a mammalian T cell. Upon the scFv binding to an antigen, the signaling domain initiates a cascade of events that ultimately results in transcription and activation of genes. In one example, the cell is further modified with a construct that expresses a marker protein, such as a fluorescent protein, luminescent protein, enzyme, or selectable marker that allows differentiation between that cell and other non-activated cells in the population. Thus, a population of cells containing a library of antibody constructs can be screened for those cells which are activated by binding to a target.

### Immune Binding Protein and Antigens

Immune binding proteins bind a very diverse spectrum of antigens, with varying levels of affinity and specificity. In some embodiments, immune binding proteins bind very specific antigens, while other immune binding proteins bind a broader array of antigens. Depending on the application, either one of these options may be desired. For example, an immune binding protein that can recognize multiple strains of influenza would have benefit against may strains of influenza, whereas an immune binding protein for an anti-tumor therapy may need to bind only one very specific conformation of an antigen, to avoid attacking normal versions of the antigen present on healthy cells and tissues.

In some embodiments, a repertoire of immune binding proteins (e.g., antibodies, T-cell receptors, and/or innate immunity receptors) made by the methods of the invention is screened against a panel of antigens. In some embodiments, each member of the panel of antigens is labeled with nucleic acids encoding unique bar codes for each antigen. In some embodiments, the screening of multiple antigens is followed by amplification reactions that produce nucleic acids encoding the polypeptide chains of the immune binding protein (e.g., the heavy and light chains of an antibody) and the antigen (e.g., if the antigen is a polypeptide) or a nucleic acid bar code for the antigen. In some embodiments, immune binding proteins are displayed on a cell surface and screened against a panel of bar-coded antigens. Those cells with displayed immune binding proteins that bind an antigen are place in microwells (single cell in each microwell) and/or capture in an emulsion, and amplification reactions are performed to make nucleic acids encoding the chains of the immune binding protein and the bar code of the antigen.

In some embodiments, an amplification reaction as describe above for an immune protein is used adding a set of forward and reverse primers for amplification of the nucleic acid attached to the antigen (AF and AR) (FIG. 1C). In some embodiments, the AR primer additionally contains a barcode (BC5) and an OE region matching that of a primer for a nucleic acid encoding one of the chains of the immune protein (e.g., the LF primer for an antibody). The amplification is carried out, resulting in a mixture of nucleic acids encoding the immune protein (e.g., HC/LC molecules) and nucleic acids encoding a chain of the immune protein and the nucleic acid for identifying the antigen (e.g., HC/Antigen molecules). In some embodiments, these molecules are sequenced using high-throughput methods, and the resulting information identifies antigens with individual immune binding proteins (e.g., antibodies).

In some embodiments, a second overlap extension (OE) is placed on the BR and immune protein primers (e.g., for an antibody the LF primer). In this embodiment, following amplification one obtains a nucleic acid encoding the chains for the immune binding protein (e.g., heavy and light chains of an antibody), and the bar code for the antigen. In some embodiments, this multipartite nucleic acid is sequenced to identify the immune binding protein, and the antigens to which the immune binding protein bound.

### Nucleic Acids

In some embodiments, the present invention relates to the nucleic acids that encode, at least in part, the individual peptides, polypeptides, proteins, and RNA control devices of the present invention. In some embodiments, the nucleic acids may be natural, synthetic or a combination thereof. The nucleic acids of the invention may be RNA, mRNA, DNA or cDNA.

In some embodiments, the nucleic acids of the invention also include expression vectors, such as plasmids, or viral vectors, or linear vectors, or vectors that integrate into chromosomal DNA. Expression vectors can contain a nucleic acid sequence that enables the vector to replicate in one or more selected host cells. Such sequences are well known for a variety of cells. The origin of replication from the plasmid pBR322 is suitable for most Gram-negative bacteria. In eukaryotic host cells, e.g., mammalian cells, the expression vector can be integrated into the host cell chromosome and then replicate with the host chromosome. Similarly, vectors can be integrated into the chromosome of prokaryotic cells.

Expression vectors also generally contain a selection gene, also termed a selectable marker. Selectable markers are well-known in the art for prokaryotic and eukaryotic cells, including host cells of the invention. Generally, the selection gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli. In some embodiments, an exemplary selection scheme utilizes a drug to arrest growth of a host cell. Those cells that are successfully transformed with a heterologous gene produce a protein conferring drug resistance and thus survive the selection regimen. Other selectable markers for use in bacterial or eukaryotic (including mammalian) systems are well-known in the art.

In some embodiments, an example of a promoter that is capable of expressing a transgene encoding an immune binding protein of the invention in a mammalian host cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving expression from transgenes cloned into a lentiviral vector. See, e.g., Milone et al., Mol. Ther. 17(8): 1453-1464 (2009), which is incorporated by reference in its entirety for all purposes. Another example of a promoter is the immediate early cytomegalovirus (CMV) promoter sequence. This promoter sequence is a strong constitutive promoter sequence capable of driving high levels of expression of any polynucleotide sequence operatively linked thereto. Other constitutive promoter sequences may also be used, including, but not limited to the simian virus 40 (SV40) early promoter, mouse mammary tumor virus promoter (MMTV), human immunodeficiency virus (HIV) long terminal repeat (LTR) promoter, MoMuLV promoter, phosphoglycerate kinase (PGK) promoter, MND promoter (a synthetic promoter that contains the U3 region of a modified MoMuLV LTR with myeloproliferative sarcoma virus enhancer, see, e.g., Li et al., J. Neurosci. Methods vol. 189, pp. 56-64 (2010) which is incorporated by reference in its entirety for all purposes), an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter. Further, the invention is not limited to the use of constitutive promoters.

Inducible promoters are also contemplated as part of the invention. Examples of inducible promoters include, but are not limited to a metallothionine promoter, a glucocorticoid promoter, a progesterone promoter, a tetracycline promoter, a c-fos promoter, the T-REx system of ThermoFisher which places expression from the human cytomegalovirus immediate-early promoter under the control of tetracycline operator(s), and RheoSwitch promoters of Intrexon. Karzenowski, D. et al., BioTechiques 39:191-196 (2005); Dai, X. et al., Protein Expr. Purif 42:236-245 (2005); Palli, S. R. et al., Eur. J. Biochem. 270:1308-1515 (2003); Dhadialla, T. S. et al., Annual Rev. Entomol. 43:545-569 (1998); Kumar, M. B, et al., J. Biol. Chem. 279:27211-27218 (2004); Verhaegent, M. et al., Annal. Chem. 74:4378-4385 (2002); Katalam, A. K., et al., Molecular Therapy 13:S103 (2006); and Karzenowski, D. et al., Molecular Therapy 13:S194 (2006), U.S. Pat. Nos. 8,895,306, 8,822,754, 8,748,125, 8,536,354, all of which are incorporated by reference in their entirety for all purposes.

Expression vectors of the invention typically have promoter elements, e.g., enhancers, to regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

In some embodiments, control regions suitable for a bacterial host cells are used in the expression vector. In some embodiments, suitable control regions for directing transcription of the nucleic acid constructs of the invention, include the control regions obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and the prokaryotic beta-lactamase gene, the tac promoter, or the T7 promoter.

In some embodiments, control regions for filamentous fungal host cells, include control regions obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid control regions thereof. Exemplary yeast cell control regions can be from the genes for Saccharomyces cerevisiae enolase (ENO-1), Saccharomyces cerevisiae galactokinase (GAL 1), Saccharomyces cerevisiae alcohol dehydrogenase/glyceraldehyde-3-phosphate dehydrogenase (ADH2/GAP), and Saccharomyces cerevisiae 3-phosphoglycerate kinase.

In some embodiments, exemplary control regions for insect cells include, among others, those based on polyhedron, PCNA, OplE2, OplE1, Drosophila metallothionein, and Drosophila actin 5C. In some embodiments, insect cell promoters can be used with Baculoviral vectors.

In some embodiments, exemplary control regions for plant cells include, among others, those based on cauliflower mosaic virus (CaMV) 35S, polyubiquitin gene (PvUbi1 and PvUbi2), rice (Oryza sativa) actin 1 (OsAct1) and actin 2 (OsAct2) control regions, the maize ubiquitin 1 (ZmUbi1) control region, and multiple rice ubiquitin (RUBQ1, RUBQ2, rubi3) control regions.

In some embodiments, the expression vector contains one or more selectable markers, which permit selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers, which confer antibiotic resistance such as ampicillin, kanamycin, chloramphenicol (Example 1) or tetracycline resistance. Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Embodiments for use in an Aspergillus cell include the amdS and pyrG genes of Aspergillus nidulans or Aspergillus oryzae and the bar gene of Streptomyces hygroscopicus.

In some embodiments, it may be desirable to modify the polypeptides of the present invention. One of skill will recognize many ways of generating alterations in a given nucleic acid construct to generate variant polypeptides Such well-known methods include site-directed mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and other well-known techniques (see, e.g., Gillam and Smith, Gene 8:81-97, 1979; Roberts et al., Nature 328:731-734, 1987, which is incorporated by reference in its entirety for all purposes). In some embodiments, the recombinant nucleic acids encoding the polypeptides of the invention are modified to provide preferred codons which enhance translation of the nucleic acid in a selected organism.

The polynucleotides of the invention also include polynucleotides including nucleotide sequences that are substantially equivalent to the polynucleotides of the invention. Polynucleotides according to the invention can have at least about 80%, more typically at least about 90%, and even more typically at least about 95%, sequence identity to a polynucleotide of the invention. The invention also provides the complement of the polynucleotides including a nucleotide sequence that has at least about 80%, more typically at least about 90%, and even more typically at least about 95%, sequence identity to a polynucleotide encoding a polypeptide recited above. The polynucleotide can be DNA (genomic, cDNA, amplified, or synthetic) or RNA. Methods and algorithms for obtaining such polynucleotides are well known to those of skill in the art and can include, for example, methods for determining hybridization conditions which can routinely isolate polynucleotides of the desired sequence identities.

Nucleic acids which encode protein analogs or variants in accordance with this invention (i.e., wherein one or more amino acids are designed to differ from the wild type polypeptide) may be produced using site directed mutagenesis or PCR amplification in which the primer(s) have the desired point mutations. For a detailed description of suitable mutagenesis techniques, see Sambrook et al., Molecular Cloning: A Laboratory Manual, Cold Spring Harbor Laboratory Press, Cold Spring Harbor, N.Y. (1989) and/or Current Protocols in Molecular Biology, Ausubel et al., eds, Green Publishers Inc. and Wiley and Sons, N.Y (1994), each of which is incorporated by reference in its entirety for all purposes. Chemical synthesis using methods well known in the art, such as that described by Engels et al., Angew Chem Intl Ed. 28:716-34, 1989 (which is incorporated by reference in its entirety for all purposes), may also be used to prepare such nucleic acids.

In some embodiments, amino acid “substitutions” for creating variants are preferably the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

The nucleic acid of the present invention can be linked to another nucleic acid so as to be expressed under control of a suitable promoter. The nucleic acid of the present invention can be also linked to, in order to attain efficient transcription of the nucleic acid, other regulatory elements that cooperate with a promoter or a transcription initiation site, for example, a nucleic acid comprising an enhancer sequence, a polyA site, or a terminator sequence. In addition to the nucleic acid of the present invention, a gene that can be a marker for confirming expression of the nucleic acid (e.g. a drug resistance gene, a gene encoding a reporter enzyme, or a gene encoding a fluorescent protein) may be incorporated.

When the nucleic acid of the present invention is introduced into a cell ex vivo, the nucleic acid of the present invention may be combined with a substance that promotes transference of a nucleic acid into a cell, for example, a reagent for introducing a nucleic acid such as a liposome or a cationic lipid, in addition to the aforementioned excipients. Alternatively, a vector carrying the nucleic acid of the present invention is also useful. Particularly, a composition in a form suitable for administration to a living body which contains the nucleic acid of the present invention carried by a suitable vector is suitable for in vivo gene therapy.

### Host Cells

In some embodiments, nucleic acids encoding an immune binding protein of the invention (e.g., an antibody) are cloned into an appropriate expression vector for expression of immune binding protein in a host cell. In some embodiments, the host cells of the invention include, for example, bacterial, fungi, or mammalian host cells. In some embodiments, the host cell is a bacterium, including, for example, Bacillus, such as B. lichenformis or B. subtilis; Pantoea, such as P. citrea; Pseudomonas, such as P. alcaligenes; Streptomyces, such as S. lividans or S. rubiginosus; Escherichia, such as E. coli; Enterobacter; Streptococcus; Archaea, such as Methanosarcina mazei; or Corynebacterium, such as C. glutamicum.

In some embodiments, the host cells are fungi cells, including, but not limited to, fungi of the genera Saccharomyces, Klyuveromyces, Candida, Pichia, Debaromyces, Hansenula, Yarrowia, Zygosaccharomyces, or Schizosaccharomyces. In some embodiments, the host cell is a fungi, including, among others, Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, Rhizopus oryzae, Yarrowia lipolytica, and the like. In some embodiments, the eukaryotic cells are algal, including but not limited to algae of the genera Chlorella, Chlamydomonas, Scenedesmus, Isochrysis, Dunaliella, Tetraselmis, Nannochloropsis, or Prototheca. In some embodiments, the algae is a green algae, red algae, glaucophytes, chlorarachniophytes, euglenids, chromista, or dinoflagellates.

In some embodiments, the eukaryotic cells are mammalian cells, such as mouse, rat, rabbit, hamster, porcine, bovine, feline, or canine. In some embodiments, the mammalian cells are cells of primates, including but not limited to, monkeys, chimpanzees, gorillas, and humans. In some embodiments, the mammalians cells are mouse cells, as mice routinely function as a model for other mammals, most particularly for humans (see, e.g., Hanna, J. et al., Science 318:1920-23, 2007; Holtzman, D. M. et al., J Clin Invest. 103(6):R15-R21, 1999; Warren, R. S. et al., J Clin Invest. 95: 1789-1797, 1995; each publication is incorporated by reference in its entirety for all purposes). In some embodiments, animal cells include, for example, fibroblasts, epithelial cells (e.g., renal, mammary, prostate, lung), keratinocytes, hepatocytes, adipocytes, endothelial cells, and hematopoietic cells. In some embodiments, the animal cells are adult cells (e.g., terminally differentiated, dividing or non-dividing) or embryonic cells (e.g., blastocyst cells, etc.) or stem cells. In some embodiments, the animal cell is a cell line derived from an animal or other source.

In some embodiments, the mammalian cell is a cell found in the circulatory system of a mammal, including humans. Exemplary circulatory system cells include, among others, red blood cells, platelets, plasma cells, T-cells, natural killer cells, B-cells, macrophages, neutrophils, or the like, and precursor cells of the same. As a group, these cells are defined to be circulating eukaryotic cells of the invention. In some embodiments, the mammalian cells are derived from any of these circulating eukaryotic cells. The present invention may be used with any of these circulating cells or cells derived from the circulating cells. In some embodiments, the mammalian cell is a T-cell or T-cell precursor or progenitor cell. In some embodiments, the mammalian cell is a helper T-cell, a cytotoxic T-cell, a memory T-cell, a regulatory T-cell, a natural killer T-cell, a mucosal associated invariant T-cell, a gamma delta T cell, or a precursor or progenitor cell to the aforementioned. In some embodiments, the mammalian cell is a natural killer cell, or a precursor or progenitor cell to the natural killer cell. In some embodiments, the mammalian cell is a B-cell, or a plasma cell, or a B-cell precursor or progenitor cell. In some embodiments, the mammalian cell is a neutrophil or a neutrophil precursor or progenitor cell. In some embodiments, the mammalian cell is a megakaryocyte or a precursor or progenitor cell to the megakaryocyte. In some embodiments, the mammalian cell is a macrophage or a precursor or progenitor cell to a macrophage.

In some embodiments, a source of cells is obtained from a subject. The subject may be any living organism. Examples of subjects include humans, dogs, cats, mice, rats, and transgenic species thereof. In some embodiments, T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, any number of T cell lines available in the art, may be used. In some embodiments, T cells can be obtained from a unit of blood collected from a subject using any number of techniques known to the skilled artisan, such as Ficoll separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In an alternative aspect, the wash solution lacks calcium and may lack magnesium or may lack many if not all divalent cations. Initial activation steps in the absence of calcium can lead to magnified activation.

In some embodiments the plant cells are cells of monocotyledonous or dicotyledonous plants, including, but not limited to, alfalfa, almonds, asparagus, avocado, banana, barley, bean, blackberry, brassicas, broccoli, cabbage, canola, carrot, cauliflower, celery, cherry, chicory, citrus, coffee, cotton, cucumber, eucalyptus, hemp, lettuce, lentil, maize, mango, melon, oat, papaya, pea, peanut, pineapple, plum, potato (including sweet potatoes), pumpkin, radish, rapeseed, raspberry, rice, rye, sorghum, soybean, spinach, strawberry, sugar beet, sugarcane, sunflower, tobacco, tomato, turnip, wheat, zucchini, and other fruiting vegetables (e.g. tomatoes, pepper, chili, eggplant, cucumber, squash etc.), other bulb vegetables (e.g., garlic, onion, leek etc.), other pome fruit (e.g. apples, pears etc.), other stone fruit (e.g., peach, nectarine, apricot, pears, plums etc.), Arabidopsis, woody plants such as coniferous and deciduous trees, an ornamental plant, a perennial grass, a forage crop, flowers, other vegetables, other fruits, other agricultural crops, herbs, grass, or perennial plant parts (e.g., bulbs; tubers; roots; crowns; stems; stolons; tillers; shoots; cuttings, including un-rooted cuttings, rooted cuttings, and callus cuttings or callus-generated plantlets; apical meristems etc.). The term “plants” refers to all physical parts of a plant, including seeds, seedlings, saplings, roots, tubers, stems, stalks, foliage and fruits.

### Applications

In some embodiments, the immune binding proteins of the invention are used in therapies for infectious diseases, cancer, allergies, and autoimmune diseases. In some embodiments, the methods of the invention are used to make repertoires of immune binding proteins from subjects that have been challenged/infected with an infectious agent. In some embodiments, the immune binding proteins of the invention are used in therapies to treat subjects infected with an infectious agent. In some embodiments, the immune binding proteins of the invention are used to treat subjects with cancer or allergies. In some embodiments, the immune binding proteins of the invention are used to treat melanoma, lymphoma, leukemia and other cancers responsive to immune therapy. In some embodiments, the immune binding proteins of the invention are used to treat cancers that respond to immune checkpoint inhibitor therapy. In some embodiments, addition of exogenous immune binding protein (e.g., antibody) helps the subject's body accelerate its own immune response to a pathogen, in effect “transplanting” the immunity from one individual to another. In some embodiments, the immune binding proteins of the invention are used prophylactically. In some embodiments, the immune binding proteins of the invention are used in diagnostic applications. In some embodiments, the immune binding proteins of the invention provide information on a subject's response to a therapy. In some embodiments, the immune binding proteins of the invention provide information on a subject's response to an antibody therapy, small molecule drug therapy, biologic therapy, or cellular immunotherapy.

In some embodiments, immune binding proteins (e.g., antibodies) can be obtained from the subject that neutralize an infectious agent or can be made to become neutralizing. In some embodiments, the infectious agent is a bacterial strain of Staphylococci, Streptococcus, Escherichia coli, Pseudomonas, or Salmonella. In some embodiments, the infectious agent is a Staphylococcus aureus, Neisseria gonorrhoeae, Streptococcus pyogenes, Group A Streptococcus, Group B Streptococcus (Streptococcus agalactiae), Streptococcus pneumoniae, and Clostridium tetani. In some embodiments, the infectious agent is a bacterial pathogen that may infect host cells including, for example, Helicobacter pyloris, Legionella pneumophilia, a bacterial strain of Mycobacteria sps. (e.g. M. tuberculosis, M. avium, M. intracellulare, M. kansaii, or M. gordonea), Neisseria meningitides, Listeria monocytogenes, R. rickettsia, Salmonella spp., Brucella spp., Shigella spp., or certain E. coli strains or other bacteria that have acquired genes with invasive factors. In some embodiments, the infectious agent is a bacterial pathogen that is antibiotic resistant. In some embodiments, the infectious agent is a viral pathogen including, for example, Ebola, Zika, RSV, Retroviridae (e.g. human immunodeficiency viruses such as HIV-1 and HIV-LP), Picornaviridae (e.g. poliovirus, hepatitis A virus, enterovirus, human coxsackievirus, rhinovirus, and echovirus), rubella virus, coronavirus, vesicular stomatitis virus, rabies virus, ebola virus, parainfluenza virus, mumps virus, measles virus, respiratory syncytial virus, influenza virus, hepatitis B virus, parvovirus, Adenoviridae, Herpesviridae [e.g. type 1 and type 2 herpes simplex virus (HSV), varicella-zoster virus, cytomegalovirus (CMV), and herpes virus], Poxviridae (e.g. smallpox virus, vaccinia virus, and pox virus), or hepatitis C virus.

In some embodiments, immune binding proteins of the invention are used to boost the immunity of a subject against an infectious disease. For example, in influenza the body responds within 7-10 days to a challenge; however, in immunocompromised patients such as the elderly, the immune response timing or extent may be insufficient to fight off the infection, resulting in severe complications and possibly death. By boosting the immune system with antibodies designed to fight the relevant strain of influenza, the infection in the subject can treated. In some embodiments, the methods of the invention are used to rapidly develop strain-specific antibodies to emerging pandemic strains of influenza. In some embodiments, immune binding proteins of the invention are used to treat infected patients and/or passively immunize vulnerable populations facing an outbreak. In some embodiments, the immune binding proteins are administered prophylactically. In some embodiments, the prophylactic administration of the immune binding proteins protect at risk groups of subjects from a disease.

In some embodiments, the infectious agent is a herpes simplex virus 1 (HSV-1), herpes simplex virus 2 (HSV-2), varicella zoster, Epstein-Barr, cytomegalovirus (CMV), or Kaposi's sarcoma viruses. HSV-1 primarily causes oral herpes, ocular herpes, and herpes encephalitis, and occasionally causes genital herpes; HSV-2 primarily causes genital herpes but can also cause oral herpes; varicella zoster causes chickenpox and shingles; Epstein-Barr causes mononucleosis and is associated with several cancers including Burkitt's lymphoma; CMV causes mononucleosis-like syndrome and congenital/neonatal morbidity and mortality. Some of the herpesviridae, and in particular HSV-1, have been associated with and proposed as causative agents for Alzheimer's Disease. In some embodiments, immune binding proteins of the invention can be used to treat and/or passively immunize against these herpesviridae. In some embodiments, an injection or topical application of an antibody against HSV-1 or HSV-2 can be employed to reduce the incidence or severity of the effects of herpes outbreaks.

In some embodiments, the immune binding proteins of the invention are useful for treating a cancer. In some embodiments, the cancer is a sarcoma, carcinoma, melanoma, chordoma, malignant histiocytoma, mesothelioma, glioblastoma, neuroblastoma, medulloblastoma, malignant meningioma, malignant schwannoma, leukemia, lymphoma, myeloma, myelodysplastic syndrome, myeloproliferative disease. In some embodiments, the cancer is a leukemia, lymphoma, myeloma, myelodysplastic syndrome, and/or myeloproliferative disease. In some embodiments, the cancer is one that is responsive to immunotherapy. In some embodiments, the cancer is one that is responsive to immune checkpoint inhibitor therapy.

In some embodiments, the immune binding proteins of the invention are specific for a tumor specific or enriched antigen. In some embodiments, examples of tumor specific or enriched antigens include, for example, one or more of 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD19, CD20, CD21, CD22, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, EphA3, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG, L1-CAM, IL-13, IL-6, insulin-like growth factor 1 receptor, alpha 5β1-integrin, integrin αvβ3, MORAb-009, MS4A1, MUC1, mucin CanAg, N-glycolylneuraminic acid, NPC-1C, PDGF-Rα, PDL192, phosphatidylserine, prostatic carcinoma cells, RANKL, RON, ROR1, SCH 900105, SDC1, SLAMF7, TAG-72, tenascin C, TGF β2, TGF-β, TRAIL-R1, TRAIL-R2, tumor antigen CTAA16.88, VEGF-A, VEGFR-1, VEGFR2, 707-AP, ART-4, B7H4, BAGE, β-catenin/m, Bcr-abl, MN/C IX antibody, CAMEL, CAP-1, CASP-8, CD25, CDC27/m, CDK4/m, CT, Cyp-B, DAM, ErbB3, ELF2M, EMMPRIN, ETV6-AML1, G250, GAGE, GnT-V, Gp100, HAGE, HLA-A*0201-R170I, HPV-E7, HSP70-2M, HST-2, hTERT (or hTRT), iCE, IL-2R, IL-5, KIAA0205, LAGE, LDLR/FUT, MAGE, MART-1/melan-A, MART-2/Ski, MCIR, myosin/m, MUM-1, MUM-2, MUM-3, NA88-A, PAP, proteinase-3, p190 minor bcr-abl, Pml/RARα, PRAME, PSA, PSM, PSMA, RAGE, RU1 or RU2, SAGE, SART-1 or SART-3, survivin, TPI/m, TRP-1, TRP-2, TRP-2/INT2, WT1, NY-Eso-1 or NY-Eso-B or vimentin.

In some embodiments, the tumor antigen-binding immune binding protein (e.g., antibody) can be used to make a chimeric antigen receptor specific for the tumor antigen and this CAR construct is placed into a T cell and/or a natural killer cell. In some embodiments the T-cell and/or natural killer cells with the tumor specific CAR are used to treat subjects with cancers that bear the tumor antigen.

In some embodiments, the immune binding proteins of the invention are useful for treating subjects with allergies. Common allergens include shellfish, nuts, milk, ollen, certain medications, latex, insect bites, and some plant compounds (e.g. urushiol). In some embodiments, the immune binding proteins of the invention bind the allergen of interest without triggering the allergic reaction. For example, the immune binding protein could be an antibody without an Fc region or could be an antibody in an IgG format or other format that is not an IgE format. In these embodiments, the immune binding protein of the invention binds to the allergen without triggering an allergic reaction and this binding can prevent IgE antibody in the subject from binding to the allergen and causing the allergic reaction (this is a competitive inhibition reaction). In some embodiments, the immune binding protein which binds the allergen is obtained from the subject with the allergy.

In some embodiments, the immune binding proteins of the invention are useful for treating subjects with autoimmune diseases. In some embodiments, the autoimmune disease is rheumatoid arthritis, lupus, celiac disease, Sjorgren's syndrome, polymyalgia rheumatica, multiple sclerosis, ankylosing spondylitis, Type 1 diabetes, and the like. In some embodiments, the immune binding proteins of the invention bind the antigen target of the autoimmune disease without triggering the autoimmune reaction. For example, the immune binding protein could be an antibody without an Fc region, or could be an antibody in a format that does not interact with the effector cells that are associated with the autoimmune disease. In these embodiments, the immune binding protein of the invention binds to the autoimmune antigen without triggering an autoimmune reaction and this binding can prevent the subject's immune system from reacting with the autoimmune antigen reducing the autoimmune disease (this can be a competitive inhibition reaction).

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

### EXAMPLES

### Example 1. Multiplexed Antigen Staining of Primary Cells

In some embodiments, barcoded peptide antigens are prepared by incubating antigens with an NHS DBCO heterobifunctional crosslinker. Secondly a DNA oligo with a 5′ primer site, a DNA barcode, a 3′ primer site, a 3′ poly dt, and containing a 3′ biotin and a 5′ azide are mixed with the peptide-DBCO antigens to make bar code labeled antigens.

In some embodiments, human B cells with membrane bound receptors are isolated using magnetic separation. Cells are incubated with the mixture of bar code labelled antigens so that labelled antigens bind membrane bound immunoglobulin receptors. The cells are washed and optionally the cells may be FACS sorted after incubating them with a streptavidin-PE fluorophore. In some embodiments, the cells are then encapsulated into a core shell bead containing a Triton based lysis mixture and poly-dt primer with a 5′ amplification tag. In some embodiments, a reverse transcription reaction is performed with a template switching reverse transcriptase, a template switch primer and appropriate buffer and dNTP mixture. The cDNA library with barcoded antigen is amplified with KAPA Hifi and primers specific to the amplification tag and the template switch sequence. In some embodiments, specific regions of interest, such as the heavy and light chain CDR regions and the antigen barcode, are amplified with primers containing a well-specific barcode and a 3′ primer to the region of interest via PCR. In some embodiments, these fragments are used to generate a sequencing library for high throughput sequencing. After sequencing, the data is de-convoluted by identification of core-shell bead specific barcodes, sequence assembly of heavy and light chain reads and identification of reads with antigen barcodes.

### Example 2. Multiplexed Antigen Library Sequencing Using Beads

A pool of B-cells bound to antigens is made as described in Example 1. In some embodiments, following antigen staining and washing, cells are encapsulated into core-shell beads. In some embodiments, the core of the bead comprises lysis/binding mix containing one or more barcoded poly-dt capture beads (beads coated with a DNA primer containing a 5′ amplification tag and a 3′ poly dT sequence) in a high salt/detergent buffer and 1-10 cells. As the cells lyse, their RNA is captured on the barcoded poly-dt beads as is the barcoded antigen DNA. In some embodiments, the emulsion is broken under stringent binding conditions, such as with methylene chloride and 6×SSC buffer. The bead mixture is washed twice and resuspended in a reverse transcriptase reaction and incubated. In some embodiments, the beads (“capture beads”) are separated in another water/oil emulsion generated with a monodisperse droplet generator so that each droplet has about one “template bead” in a PCR mixture. The PCR mixture also contains one or more “prep” beads containing beads that are coated with primers containing a 5′ amplification tag and a bead specific barcode. In some embodiments, the primers have a 3′ poly dA, some have a 3′ antigen primer, some have a 3′ heavy chain reverse primer, and some have a 3′ light chain reverse primer. In some embodiments, the aqueous phase has 5′ heavy chain primers, 5′ light chain primers, 5′ antigen primers and the 5′ amplification tag from the poly dT capture beads. Kapa Hifi is a suitable polymerase for this amplification. In some embodiments, following PCR the emulsion is broken and a high throughput sequencing library is generated. Following sequencing, all reads associated with the last round PCR barcodes are split into pools. Then, cell-specific barcodes are identified by the reads associated with the polyA/5′ amplification tag. In some embodiments, all reads associated with beads containing the same cell-specific barcodes are grouped together. In some embodiments, these groups are used to provide the sequence or identification of the heavy chain, light chain and the antigen which associate together.

### Example 3. Multiplexed Antigen Library Sequencing Using 5′5′ Primers

A 5′5′ primer is made by mixing a 5′ DBCO oligonucleotide and a 5′ azide oligonucleotide. In some embodiments, the DBCO and azide do not need to be at the precise 5′ end of the component oligos but may be placed in a manner that still allows for the 3′ end to perform a PCR reaction. The combined product is isolated from unreacted component oligos. In some embodiments, it may be higher yielding to use these 5′5′ primers instead of beads for linking reads to cell-specific barcodes. In some embodiments, a reaction uses primers containing a 5′5′ linkage with one of the 3′ ends containing a polyA and the other containing a 3′ light, 3′ heavy or 3′ antigen tag. In some embodiments, the reaction mix also contains 5′ heavy, 5′ light and 5′ antigen and 5′ amplification tag primers with 5′ phosphate groups. In some embodiments, nucleic acids inside a core-shell bead are incubated with a 5′5′ primer mixture and KAPA hifi in a suitable buffer with dNTP's, etc. and re-emulsified for thermal cycling. Following emulsion PCR, the emulsion is broken with methylene chloride, the aqueous phase extracted and cleaned. The DNA obtained is resuspended in ligation buffer with ligase. In some embodiments, the DNA obtained after ligation is treated with exonuclease(s). In some embodiments, the mixture obtained after exonuclease treatment is placed into a PCR with KAPA hifi for 20 cycles with the 3′ polyA primer, 3′ heavy primer and 3′ light primer. In some embodiments, the PCR product is used as a template to generate a sequencing library which is sequenced on a high throughput sequencer. Following sequencing, the reads are grouped according to their cell-specific barcode and then reads for heavy, light and antigen are identified.

### Example 4. Multiplexed Gene Specific Bead Libraries with PCR

In some embodiments, bead libraries are made where each bead has primers containing a bead specific barcode, molecule specific barcode and a plurality of gene specific primers. In some embodiments, MyOne carboxylate dynabeads are first coated with a 5′ amplification primer sequence. The beads are incubated with a limited dilution of DNA primers containing the reverse complement amplification sequence at the 3′ end, a unique molecular barcode comprising 12 N residues, and an adapter sequence of 12 bases (for example the M13 sequencing primer sequence). After incubating the beads with this mixture, the beads are pelleted and washed, and then placed in a Klenow exo-polymerase reaction. The beads are then pelleted and washed.

### Example 5. Multiplexed Gene Specific Bead Libraries with Ligation

In some embodiments, bead libraries are made where each bead has primers containing a bead specific barcode, molecule specific barcode and a plurality of gene specific primers. In some embodiments, MyOne carboxylate dynabeads are first coated with a 5′ amplification primer sequence with a 5′ amino moiety. The beads are then incubated with a limited dilution of DNA primers containing the reverse complement amplification sequence at the 3′ end, a unique molecular barcode comprising 12 N residues, and an adapter sequence of 12 bases (for example the M13 sequencing primer sequence). After incubating the beads with this mixture, the beads are treated with Klenow exo-polymerase. In some embodiments, the beads are then mixed with a soluble version of the reverse complement adapter sequence and placed into core shell beads. Following core shell generation, the emulsion is cycled for 30× and then broken. The beads are placed in a mixture with double stranded DNA sequence with the forward strand containing a 5′ phosphate, 10 base random DNA sequence, and the 3′ heavy primer, 3′ light chain primer or 3′ antigen tag primer at the 3′ end. The mixture also contains T4 DNA ligase. After this reaction, the beads are treated with T7 exonuclease.

### Example 6. Preparation of B Cells with Membrane Bound Receptors

In some embodiments, it may be beneficial to increase the receptor density on cells. In some embodiments, primary B cells are transformed into antibody secreting plasma cells by incubation with IL21, IL4, and CD40L. These cells are treated with an NHS-azide heterobifunctional crosslinker. Protein-G DBCO is prepared by mixing protein G with an NHS-DBCO heterobifunctional crosslinker. The cells are treated with the protein-G DBCO with additional protein-G and then spatially separated in core shell beads with soluble or solid phase protein-G in the buffer. The cells are removed from the core shell bead by dissolution of the bead and placed in a solution with a metabolic inhibitor such as present in many commonly available stain buffers. Following this treatment, the cells are reacted with antigens.

### Example 7. Preparation of B Cells with Hydrogel Bound Receptors

In some embodiments, it may be beneficial to further increase the receptor density on the antigen binding cells. In some embodiments, primary B cells are transformed into antibody secreting plasma cells by incubation with IL21, IL4, and CD40L. The cells are treated with an NHS-azide heterobifunctional crosslinker and then isolated in core-shell beads. The cells in the microwells are treated with an DBCO 4× dendrimer PEG, and then treated with an azide-azide homobifunctional 1 kd PEG. In some embodiments, the DBCO 4× dendrimer PEG treatment and the homobifunctional azide-azide 1 kda PEG treatment are repeated for a desired number of rounds. These additional cycles of DBCO/azide pegs create additional functionalization sites and larger hydrogel volume for better signal until a desired amount of functionalization and/or hydrogel is produced. In some embodiments, Protein-G DBCO is prepared by mixing protein G with an NHS-DBCO heterobifunctional crosslinker. The cells embedded in hydrogel are treated with the protein-G DBCO with additional protein-G. The cells are released by dissolution of the core shell bead from the microwell and placed in a solution with a metabolic inhibitor such as present in many commonly available stain buffers. The cells are ready for reaction with antigens. Alternatively, the cell/hydrogel mixture is left in the core shell and stained in situ with antigens.

### Example 8. Preparation of B Cells with Magnetic Bead Bound Receptors

In some embodiments, primary B cells are transformed into antibody secreting plasma cells by incubation with IL21, IL4, and CD40L. The cells are treated with an NHS-azide heterobifunctional crosslinker and washed.

In some embodiments, Protein-G beads are prepared by activating magnetic carboxylated beads with EDC/sulfo NHS and reacting with protein G. Protein-G DBCO beads are prepared by mixing protein G beads with an NHS-DBCO heterobifunctional crosslinker. The cells are spatially separated in core shell beads with Protein G DBCO beads. In some embodiments, soluble azide PEG and soluble protein G is also added to the beads following de-emulsification. In some embodiments, beads with antibodies are separated from core-shells following dissolution of the core shell. The antibody beads are then reacted with antigen. Alternatively, the cell/bead mixture is left in the core shell bead and stained in situ with antigens.

### Example 9. Multiplexed ScFv Generation Using 5′5′ Primers

In some embodiments, cDNA made from individual cells as described above is isolated in a core shell bead in a mixture containing a library of linked 5′5′ primers, where one side is specific to the 5′ coding frame of the heavy chain variable sequence and one side is specific to the 3′ coding frame of a light chain variable sequence. Additionally, the PCR mix contains Kapa Hifi polymerase and a primer library for light chain 5′ variable regions and heavy chain 3′ variable regions. The DNA obtained from the reaction is ligated with T4 ligase and then treated with exonuclease. This mixture is placed into a PCR with KAPA hifi for 20 cycles with the 3′ heavy primer library and 5′ light primer library. Following PCR this material is cloned into a suitable expression vector for production of proteins containing an ScFv fragment. Alternatively, or in addition the combined ScFv DNA library is used to make a sequencing library for high throughput sequencing.

### Example 10. Microfluidic System for Making Gel-Beads

A microfluidic device is used to generate water/oil emulsions (droplets), which are subsequently polymerized into gel-beads. A core aqueous solution, which contains gelation reagent(s), such as agarose, PEG and/or polyacrylamide, is provided in a channel that contains the solution. As the core solution flows through the device it is subjected to a laminar flow channel of oil to create a water in oil emulsion. After the emulsion droplets are formed, the gelation reagent is activated by subjecting the gelation reagent in the droplets to light, temperature change, and/or an ion or free radical. The gel-beads are rapidly formed and then collected.

### Example 11. Microfluidic System for Making Core-Shell Beads

A microfluidic device is used which device has two (2) laminar cross flow channels that flow across a core aqueous solution channel. A first laminar cross flow channel contains a gelation reagent monomer solution. A second cross flow channel contains oil. The channel with the core fluid is first subjected to a laminar (cross flow) of a fluid with the gelation reagent. This forms a column of fluid with the cored solution in the middle and the gelation solution on the outside of the column. This column of solution flows through the channel and is subjected to a second laminar (cross flow) of an oil. The oil causes a water in oil emulsion to form, where the droplets of the emulsion have a center with the core solution and an outer layer with the gelation monomer. These droplets, once formed, are treated to rapidly polymerize the monomer so as to form a core (liquid) and shell (gel) droplet. The monomer can be polymerized by, for example, light, temperature change, an ion, or a free radical. The core-shell beads are rapidly formed and then collected.

### Example 12. Microfluidic System for Making Core-Shell Beads Having a Stabilizing Membrane

Core-shell beads are formed as described in Example 11. In this example, the formed droplets include a stabilizing membrane to protect the droplets. The stabilizing membrane can be a nylon membrane, which can be created by placing one monomer of the membrane in the core solution, and the other monomer in the oil phase. When the water in oil emulsion (droplets) form a nylon membrane at the interphase between the two fluids can form as the monomers of the membrane are able to react at the interphase. This forms a membrane that can maintain the droplets until the gel is formed. The monomers of the membrane can optionally include functional groups which allow the membrane polymer to be broken through a subsequent reaction. Such functionally groups include, for example, disulfides, which are later removed through a reducing environment. Other functional groups, as described above, include linkers, which can be broken and removed by a protease, and also nucleotides, which can be broken and removed by a nuclease.

### Example 13. Biomolecule Capture in a Core-Shell Bead

The composition of the gel bead is modulated to prevent diffusion of large biomolecular targets (e.g., genomic DNA) or adducts (e.g., RNA bound to a polymer scaffold), while allowing diffusion of solvents, salts, small molecules, and small biomolecules (e.g., enzymes). One or more biomolecule capture scaffolds can be included during core shell bead synthesis. The scaffold includes one or more capture reagents that bind to targets in the core solution. A scaffold can be formed of polyacrylamide by using monomers to which target capture agents (e.g., oligonucleotides) are attached. These monomers polymerize into a gel/scaffold with the target capture agents (e.g., oligonucleotides, protein G) attached for capture of target molecules (e.g., mRNA, antibodies, respectively). Alternatively, a scaffold is made using ferromagnetic or polymer beads functionalized with chemical moieties that enable attachment of biomolecular targets (e.g., poly dT magnetic beads). Alternatively, a scaffold is made using a polymer with biomolecule capture moieties that is unable to diffuse rapidly through the shell of the bead and is included in the shell or in the core solution. The target molecules are released from cells in the core and optionally captured on the capture scaffold.

### Example 14. Cell Encapsulation and Inside a Core Shell Bead

One or more cells are encapsulated in the core shell bead before shell gelation.

### Example 15. Cell Culture Inside a Core Shell Bead

Living cells are encapsulated in the bead with an appropriate cell culture medium in a manner that enables the cells to survive (e.g., 37° C., 10% CO₂ and appropriate growth factors for HEK 293F cells). Biomolecules produced by the cell may be captured on an optional biomolecule capture scaffold. The living cells can be imaged to assess viability or other functional outcomes of reagents introduced with the cells.

### Example 16. Cell Lysis Inside a Core Shell Bead

A cell lysis buffer is introduced into the core-shell bead. The cell lysis mix may be included during core-shell polymerization or introduced subsequently (e.g., after de-emulsification). When cells are mixed with the cell lysis mix, biomolecules are released from the cell and optionally captured on a biomolecule capture scaffold.

### Example 17. Capture of Proteins and mRNA from Cells

A capture scaffold with moieties specific to proteins and mRNA, respectively, and single cells are placed into core-shell beads. Antibodies produced by the cell are captured on a scaffold with Protein G. The cells are lysed and mRNA is captured on a poly dt scaffold. The combined scaffold is screened for its ability to bind targets with its captured antibodies, possibly after release of the scaffold via dissolution of the core-shell bead. Reverse transcription, DNA amplification, and sequencing is used to determine the antibody sequence.

### Example 18. Reverse Transcription

Reverse transcription reagents are introduced into the core-shell bead to enable cDNA synthesis. The template for reverse transcription may be a molecule included during core-shell polymerization, an RNA released from a cell through cell lysis, or RNA from a virus. The template may also have been captured on a scaffold as in Example 13. For instance, after cell lysis as in Example 13, and capture of the target molecules (e.g., mRNA) the target molecules can be subjected to reactions (e.g., mRNA can be reverse transcribed). Primers used for reverse transcription may have DNA or RNA barcodes on them and be either gene specific of poly dt. Reverse transcription reagents can be introduced into the core-shell bead during core-shell bead synthesis or introduced subsequently after de-emulsification of the core-shell beads. Reverse transcription may occur directly on a biomolecule attached to its molecular capture scaffold (e.g., poly dt beads). When reverse transcription reagents are introduced subsequently, the pore sizes of the core-shell polymer are tuned to enable reagents to diffuse in to the bead but prevent diffusion of large biomolecules and biomolecules attached to the capture scaffold inside.

### Example 19. DNA Polymerization in a Core Shell Bead

DNA polymerization reagents are introduced into the core-shell bead to enable DNA synthesis. The template for DNA polymerase may be a genomic DNA, a molecule included during core-shell polymerization, a PCR amplicon, a plasmid, or viral DNA. DNA polymerization reagents can be introduced into the core-shell bead during core-shell bead synthesis or introduced subsequently. For instance, following reverse transcription using poly dT primers or gene specific primers as in Example 17, the core shell beads are washed with a buffer containing enzymatic DNA polymerization reagents (5′ and/or 3′ primers, polymerase, dNTP's and suitable buffers). The pore sizes of the core-shell polymer are tuned to enable DNA polymerization reagents to diffuse in to the bead but prevent diffusion of large biomolecules and biomolecules attached to the capture scaffold inside. DNA polymerization then occurs under appropriate temperature control (e.g., anneal/extend/denature for thermostable enzymes or constant temperature for isothermal amplification).

### Example 20. Preventing Diffusion During Reactions in a Core-Shell Bead

Depending on the size of biomolecules inside a core shell bead or generated during polymerization in Examples 14 and 15, the core shell beads are re-emulsified, captured on a micropatterned surface, or confined in a microwell device, and then subjected to reaction conditions necessary for DNA polymerization (e.g., denaturation/anneal extension for a thermal stable polymerase or constant temperature for isothermal reactions). This prevents diffusion of biomolecules between core shell beads.

### Example 21. Functional Multi-Cell Assay

A library of cells or viral particles are co-encapsulated with target cells. For example, single members from a library of yeast secreting different antibody variants are co-encapsulated with a single human cell. Functional outcomes, such as target cell survival or growth are measured via imaging or cytometry. In some instances, it is necessary to place core-shell beads on a surface or into microwell arrays in order to image and select positive targets for further characterization. Positive outcomes are isolated using fluorescent cytometry or micro manipulated pipettes.

### Example 22. Functional Multi-Cell Assay with Sequencing as a Read Out

A library of cells or viral particles are co-encapsulated with target cells. For example, a library of yeast secreting different antibody variants. By inclusion of a DNA barcoded scaffold, DNA and/or transcripts of the target cell are captured along with DNA and/or transcripts of the secreting cell. Following DNA amplification (Examples 18 and 19), a sequencing library is made that contains antibody sequences and target cell transcripts with the same barcode. Following sequencing and correspondence of antibody sequences to functional outcomes (e.g., increase in a transcript level of target cell, or inhibition of target cell growth).

### Example 23. Viral Neutralization Assay Using Core Shell Beads and Micro Well Devices

A library of protein secreting cells is encapsulated into core-shell beads and cultured as in Example 15. This library of core-shell encapsulated cells is placed on a microwell array containing cells that are susceptible to viral infection such that the core shell beads are approximately the size of the microwells and register in wells in a one to one manner. The core shells are dissolved freeing the antibody into a microwell. Solution containing virus is introduced to the microwell array and the cells monitored for viability using imaging. Wells containing cells that survive are aspirated with a micro manipulated pipette and genes amplified for the protein secreting cell, which can be then identified with DNA sequencing.

### Example 24. Barcoding of Transcripts from Single Cells

A cell and a capture scaffold containing a plurality of molecules having the same DNA barcode are encapsulated during core shell bead synthesis, in a way that most core-shell beads in a mixture have different DNA barcode sequences present on the scaffold, but every scaffold within a core-shell bead has nearly the same DNA barcode sequence. The capture molecules on the capture scaffold have a gene specific primer and/or poly DT primer that is used during reverse transcription and/or PCR. Following examples (13, 14, 18 and 19 using the DNA barcoded sequence as the capture probe) all transcripts from single cells are barcoded with the same DNA barcode during templated DNA polymerization with the target molecules as templates.

### Example 25. DNA Barcoding of Nucleic Acid Templates

A nucleic acid and a capture scaffold containing a plurality of molecules having the same DNA barcode are encapsulated during core shell bead synthesis in a way that most core-shell beads in a mixture have different DNA barcode sequences present on the scaffold, but every scaffold within a core-shell bead has nearly the same DNA barcode sequence. The capture molecules on the capture scaffold have a primer that is used during templated nucleic acid synthesis, thus linking the nucleic acid sequence to the barcode sequence present in the core-shell bead. The nucleic acid template could be from a free molecule of DNA, a virus, a cell, liposome, or a nucleic acid conjugate (e.g., a protein antigen crosslinked to a DNA barcode). In an ideal embodiment, the DNA template is present in a phage with a surface displayed antigen, or a protein conjugated to a DNA molecule using Azide-DBCO click chemistry and is specific to a surface protein on an encapsulated cell. Following Examples (13, 14, 18 and 19 using the DNA barcoded sequence as the capture probe) all transcripts from single cells are barcoded with the same DNA barcode during templated DNA polymerization with the target molecules as templates, and any nucleic acids that may also be present in the mixture are also barcoded with the same barcode. Thus, DNA sequencing of a broken and pooled mixture of core-shell beads can be used to deconvolute which RNA transcripts are associated with which other nucleic acid molecules were present in any given core-shell bead.

### Example 26. DNA Barcoding within a Core-Shell Bead

A plurality of molecules inside a core-shell bead is labelled with subsequent rounds of polymerase extension through combinatorial synthesis. Nucleic acid molecules inside a core-shell bead are barcoded by splitting the solution of de-emulsified core-shell beads into multiple wells and extending the molecules inside each well with a different DNA primer specific to a given well. The DNA primer has a region that overlaps with the nucleic acid inside the core shell, and polymerase, dNTP's, suitable buffer and thermal cycle are used to enable templated DNA synthesis. After performing the first barcoding extension, the core-shell beads are pooled together and split into multiple wells again before being extended with another DNA primer specific to each well. In this manner a DNA barcode is “built-up” inside the core shell bead. In this case, 384 different DNA barcodes are used in the first step and 384 in the second to allow for up to 147456 distinct combinations. The built-up DNA barcode may be synthesized on/in the gel shell, on a capture scaffold, directly on target molecules captured on a capture scaffold, or on other large molecules that are incapable of diffusing through the shell of the bead.

### Example 27. DNA Barcoding and Combinatorial Synthesis within a Core-Shell Bead

It is desirable to perform other chemical reactions that are specific to a given core-shell bead and capture an order of operations using DNA barcoding. Gel beads containing a polymer with a capture scaffold are generated that allows addition of azide reactive chemical moieties. Beads are split and placed in a solution of azide reactive chemical moieties attached to a functional chemical moiety, where each well corresponds to a different functional chemical moiety. Each well is then washed and DNA barcoded as in Example 26 so that each bead receiving a given functional chemical moiety receives the same DNA barcode during polymerase extension. Subsequently, beads are pooled together and split for another round of chemical functionalization (e.g., in this round with an amine-reactive chemical moiety) and corresponding DNA barcoding.

### Example 28. Overlap Extension Assembly Inside a Core-Shell Bead

As in Example 19, polymerase is used to perform templated polymerization using molecules inside the core shell as templates. Molecules inside the gel bead have overlaps with each other that enables them to prime and extend off from each other, subsequently creating a fusion of two or more DNA molecules.

### Example 29. Overlap Extension of ScFv Fragments

As in Examples 18, 19 and 28, single cells containing heavy and light chain mRNA transcripts are lysed, mRNA transcripts amplified into cDNA via RT and PCR with primers that contain a suitable linker for ScFv generation (e.g., having the ability for heavy and light chain PCR products to prime and extend off of each other and have sufficient length and codon composition to code into a functional ScFv when placed into a suitable expression vector), and heavy and light chains stitched together using overlap extension PCR and a DNA linker compatible with binding sites for the heavy and light chains.

### Example 30. Overlap Extension of Alpha/Beta TCR Fragments

As in Examples 18, 19 and 28, single cells containing heavy and light chain mRNA transcripts are lysed, mRNA transcripts amplified into cDNA via RT and PCR with primers that contain a suitable linker for single chain TCR generation (e.g., having the ability for alpha and beta chain PCR products to prime and extend off of each other and have sufficient length and codon composition to code into a functional single chain TCR when placed into a suitable expression vector), and alpha and beta chains stitched together using overlap extension PCR and a DNA linker compatible with binding sites for the heavy and light chains.

### Example 31. Generation of Core-Shell Beads Using Dissolvable Gel Beads

One or more reversibly crosslinked gel beads (e.g., crosslinked with dithiol, vicinal diol, or photocleavable agent such as o-nitrobenzyl group) are encapsulated in an aqueous water/oil emulsion. The gel bead may have been synthesized as in Example 13 or other popular methods for making monodisperse gel beads. The gel bead is introduced into a microfluidic junction in an aqueous phase containing a functionalized polymer (e.g., PEG 10k-Azide-4× dendrimer) unable to diffuse deeply into the gel bead matrix because of high molecular weight exclusion and/or hydrophobic/hydrophilic interactions. A second aqueous phase is co-introduced with the gel-bead phase with a crosslinking agent (e.g., homo PEG 1k-DBCO) and additional biological materials to encapsulate. The gel bead may be functionalized to crosslink with the functionalized polymer/crosslinking agents present in either aqueous phase in order to consume gelation reagents from permeating the interior gel bead. Immediately upon mixing, the combined aqueous phases partition into a water/oil emulsion whilst subjected to a laminar flow channel of oil. The combined gel in gel bead is de-emulsified and allowed to react with the reversing agent (e.g., DTT, sodium periodate, UV light respectively) to generate an aqueous void inside the outer gel bead.

### Example 32. Bait Particle: Flu

Immune serum is isolated from healthy volunteer subjects. From these serum samples, antibodies are isolated and subsequently labeled with each serum sample bearing a unique bar code that identifies the specific subject as the source of the isolated antibodies. A plurality of bait particles is subsequently prepared having a plurality of HA antigens from several different influenza virus strains/isolates. The plurality of HA antigens (“bait particles”), are then mixed with a pool of antibodies obtained from the “X number” healthy volunteer subjects. Isolated bait particles that are paired with the binding antibodies are obtained. Subsequently, the bait particles, complete with binding antibodies, are isolated. Single bait particles are isolated through FACS to wells or can be selected using the HTS apparatus described herein to make arrays of polymer beads having single bait particles at each position. Finally, the bar codes from the single bait particles are sequenced to identify the HA isolate and the patient source of the antibody. In an alternative, specific antigens can be presented in the context of the major histocompatibility complex (“MHC”) on a bait particle, a cancer cell, or a virus infected cell to be the bait for T-cells.

### Example 33: Bait Particle: ScFv Expressing Phage

In this example, a library of ScFv expressing phage is co-incubated with a library of antigen/barcode beads. Antigen/barcode beads binding to ScFv expressing phage are subsequently co-encapsulated with a bead containing oligonucleotides with a bead specific DNA barcode and primers specific to the ScFv library. The bead specific barcode may additionally include a sequence, which is antigen specific, or alternatively, the oligonucleotides on the bead containing bead specific DNA barcodes can contain primers specific to the antigen present on the bead. Upon generation of a library, sequencing of the antigen barcode present on the bead and the corresponding ScFv sequences, identifying which ScFv's have been bound to which antigens is determined.

### Example 34: Bait Particle; MHC

A bead library is made with each bead containing all known MHC subclasses and subsequently pooled together. A first DNA barcode is present on each bead that corresponding to its respective MHC subclass. The beads are pooled together and subsequently split to be linked to a variety of peptides separately and barcoded with a second DNA barcode corresponding to the peptide on the bead. Beads are then incubated with cells/phage displaying T-cell receptor molecules (“TCR's”). The beads are then isolated, and all identified molecules are sequenced to determine the: 1) MHC barcode, 2) peptide barcode, and 3) the alpha/beta or delta/gamma chains of the TCR.

### Example 35: Bait Particle: Bacterial

Multiple strains of bacteria are crosslinked onto a bead using amine-NHS chemistry or glutaraldehyde. All strains of bacteria are suitable in the present invention, a complete list of bacterial strains may be found, for example, from the American Tissue Culture Collection (atcc.org). The beads are incubated with phage displayed ScFv's. Finally, sequencing libraries are generated employing primers for the 16S region of the bacterial genome and heavy and light chains from the ScFv's.

### Example 36: Bait Particle: Barcoded Antigen

Antigens typically utilized in an enzyme-linked immunosorbent assay (“ELISA”), are bound to a bead via a plurality of DNA oligonucleotides encoding the same DNA barcode and containing primers specific to heavy and light chains of antibody transcripts. Cells having surface bound antibodies are incubated with the barcoded antigen beads. Single antigen beads are isolated, via ABW or other emulsion/microwell devices, with any attached cells and a combination lysis and reverse transcription buffer.

### Example 37. Bait Particle: Tumor

Blood samples are isolated from a group of identified cancer patients. Employing a bait particle containing epithelial/tumor specific antibody (“Anti-EpCAM”) and appropriate barcoded primers. From the secured blood samples, circulating/metastatic tumor cells are captured from the cancer patients' blood. mRNA content is then captured from the tumor cells. Samples from different patients are then combined and transcriptome information is obtained. The sequence analysis of this transcriptome information is subsequently used to either: 1) identify potentially important target genes for therapy; 2) Classify the tumor subtype for assigning more efficient treatment, or both options. Bait particles may also be used as a method of drug delivery or neutralizing circulating/metastatic tumor cells in patients identified with aggressive cancers.

### Example 38. Selection of Cells Secreting Antibody Protein

Human primary B cells are harvested from a patient exposed to influenza antigen. The cells are treated with appropriate growth factors and cytokines to induce plasma cell differentiation. A substrate is made with a 6 well plastic microplate containing well bottoms of 170 microns thick glass. Each well bottom is patterned with a UV cured PEG hydrogel to yield wells of 100 um×100 um×100 um. Plasma cells and beads bound to antigen are loaded onto to the device. A porous membrane is placed approximately 500 microns over the cells to facilitate washing the array. One micron fluorescent beads are placed into the array at a loading rate of approximately 5% of wells to serve as fiducial marks later. Fluorescent secondary antibodies specific to human IgG, IgA and IgM are perfused into each well. The cells/beads/wells are incubated for 1-4 hours and imaged with the automated microscope. The cells/beads/wells are washed with a PBS/BSA mixture and imaged with the automated microscope. The images from the microscope are processed to identify cell presence and morphology, fluorescence signal of bead bound antibody, location of fiducials, and the location of cells that have secreted protein signal of the desired binding profile (in this case, binding of at least one strain of influenza antigen with an IgG or IgA antibody and absence of binding to human serum albumin). The perfusion membrane is slowly retracted while adding fresh PBS-BSA medium. A cell picking worklist is generated to select cells fitting the desired binding profile. The microscope images each cell to be selected, ensuring registration of fiducials, and adjusting the expected absolute coordinates of each desired cell as needed. The Z-axis cell pipette aspirates a cell from the target position and retracts so that a receiver plate can be placed between it and the substrate with the cells. The microscope images the point of aspiration and a machine vision algorithm detects whether a cell was indeed aspirated; if no cell was aspirated the aspiration repeats. A robotic arm places a 96 well plate into position under the Z-axis cell pipette and the cell dispenses the cell into a well of the 96 well plate containing single cell lysis buffer (eg, 0.1% triton). The cell is lysed and a reverse transcriptase is added with suitable buffers (eg, SmartSeq v4) and the mixture incubated at 42 C to 50 C. A sequencing library is generated by amplification through PCR of heavy and light chain cDNA's with a multiplexed primer library capable of amplifying human antibody fragments and appropriate 5′ and 3′ tags needed for loading of molecules onto an Illumina MiSeq sequencer. Sequences of antibody fragments are compared to the binding profile and cellular presence/morphology as determined by the microscope. cDNA's harvested from single cells are used as a template in PCR to amplify and perform molecular cloning of the antibody fragments into a suitable IgG expression vector.

### Example 39. Selection of High Affinity Antibodies

Human primary B cells are harvested from a patient exposed to influenza antigen. The cells are treated with appropriate growth factors and cytokines to induce plasma cell differentiation. A substrate is made with a 6 well plastic microplate containing well bottoms of 170 microns thick glass. Each well bottom is patterned with a UV cured PEG hydrogel to yield wells of 100 um×100 um×100 um. Plasma cells and beads bound to anti-human Fc (polyclonal for IgG1-4,IgA,IgM,IgE,IgD) are loaded onto to the device. A porous membrane is placed approximately 500 microns over the cells to facilitate washing the array. One micron fluorescent beads are placed into the array at a loading rate of approximately 5% of wells to serve as fiducial marks later. The cells/beads/wells are incubated for 1-4 hours and imaged with the automated microscope. The cells/beads/wells are washed with FcBlock (BD Biosciences). The cells/beads/wells are washed with a PBS/BSA mixture and imaged with the automated microscope. The cells/beads/wells are subjected to at least one cycle of (1) incubation with a mixture of fluorescently stained antigen, and (2) imaging with the automated microscope, and (3) washing with PBS/BSA/FcBlock and (4) increasing the antigen concentration and/or changing the antigen. The images from the microscope are processed to identify cell presence and morphology, fluorescence signal of bead bound antigen with respect to cycle number and antigen exposure time, location of fiducials, and the location of cells that have secreted protein signal of the desired binding profile (in this case, binding of at least one strain of influenza antigen with an IgG or IgA antibody and absence of binding to human serum albumin). The perfusion membrane is slowly retracted while adding fresh PBS-BSA medium. A cell picking worklist is generated to select cells fitting the desired binding profile. The microscope images each cell to be selected, ensuring registration of fiducials, and adjusting the expected absolute coordinates of each desired cell as needed. The Z-axis cell pipette aspirates a cell from the target position and retracts so that a receiver plate can be placed between it and the substrate with the cells. A robotic arm places a 96 well plate into position under the Z-axis cell pipette and the cell dispenses the cell into a well of the 96 well plate containing single cell lysis buffer (eg, 0.1% triton). The cell is lysed and a reverse transcriptase is added with poly dT primer, with suitable buffers (eg, SmartSeq v4) and the mixture incubated at 42 C to 50 C. A sequencing library is generated by amplification through PCR of heavy and light chain cDNA's with a multiplexed primer library capable of amplifying human antibody fragments and appropriate 5′ and 3′ tags needed for loading of molecules onto an Illumina MiSeq sequencer. Sequences of antibody fragments are compared to the binding profile and cellular presence/morphology as determined by the microscope. cDNA's harvested from single cells are used as a template in PCR to amplify and perform molecular cloning of the antibody fragments into a suitable IgG expression vector.

### Example 40: Isolation of Neutralizing Antibodies

Human primary B cells are harvested from a patient exposed to influenza antigen. The cells are treated with appropriate growth factors and cytokines to induce plasma cell differentiation. A substrate is made with a 6 well plastic microplate containing well bottoms of 170 microns thick glass. Each well bottom is patterned with a UV cured PEG hydrogel to yield wells of 200 um×200 um×200 um and an 8 bit binary encoded fiducial marking every square mm. Plasma cells and cells susceptible to influenza virus infection are loaded onto to the device such that most wells contain one or fewer plasma cells and approximately 25 susceptible cells. A porous polyester membrane is placed directly above the wells to facilitate perfusing and washing the array. The cells are incubated for 1-4 hours and imaged with the automated microscope. The cells/wells are perfused with a mixture containing live influenza virus to obtain an MOI of approximately 1:1 for the susceptible cells and imaged 4-24 hours later. The cells/wells are washed with a PBS/BSA mixture and imaged with the automated microscope. The images from the microscope are processed to identify cell presence and morphology, location of fiducials, and the location of cells that have prevented infection of nearby cells. The perfusion membrane is slowly retracted while adding fresh PBS-BSA medium. A cell picking worklist is generated to select cells secreting neutralizing antibodies. The microscope images each cell to be selected, ensuring registration of fiducials, and adjusting the expected absolute coordinates of each desired cell as needed. The Z-axis cell pipette aspirates a cell from the target position and retracts so that a receiver plate can be placed between it and the substrate with the cells. A mechatronic gantry places a 96 well plate into position under the Z-axis cell pipette and the cell dispenses the cell into a well of the 96 well plate containing single cell lysis buffer (eg, 0.1% triton). The cell is lysed and a reverse transcriptase is added with poly dT primer, with suitable buffers (eg, SmartSeq v4) and the mixture incubated at 42 C to 50 C. A sequencing library is generated by amplification through PCR of heavy and light chain cDNA's with a multiplexed primer library capable of amplifying human antibody fragments and appropriate 5′ and 3′ tags needed for loading of molecules onto an Illumina MiSeq sequencer. Sequences of antibody fragments are compared to the binding profile and cellular presence/morphology as determined by the microscope. Sequences coding for heavy and light chains of antibody genes from each cell are synthesized by de novo DNA synthesis and cloned into an isotype specific expression cassette.

### Example 41. TCR/CAR Screening and Selection

A library of expression vectors are transfected into human cells, such as HEK 293 or Raji cells, to produce an “antigen/target library”. Alternatively an antigen/target library is generated from living cells harvested from one or more tissue biopsies. The cells are placed on a hydrogel substrate in appropriate culture media. A plurality of T cells expressing a TCR or CAR are optionally transfected with a fluorescent NFAT reporter (eg, GFP) to produce an “effector library” and co-incubated with the antigen/target library for 2 to 48 hours under appropriate culture conditions. The substrate is imaged with the automated microscope. The locations of cells present on the substrate are used to generate fiducial points. The images from the microscope are processed to identify cell presence and morphology, location of fiducials, and the location of cells that have bound target cells, killed target cells and/or exhibit reporter activation. A cell picking worklist is generated to select cells optionally exhibiting reporter fluorescence and optionally exhibiting targeted cell killing (for cytotoxic T cells) as seen through changes in cellular morphology or an Incucyte™ Caspase 3/7 or Anexin V assay. Target cells in the vicinity of the effector cell are optionally included in the worklist, either as separate aspirations, or to be picked up when aspirating the effector cell, depending on the relative location of target cells and effector cells. The microscope images each cell to be selected, ensuring registration of fiducials, and adjusting the expected absolute coordinates of each desired cell as needed. The Z-axis cell pipette aspirates one or more cells from the target position and retracts so that a receiver plate can be placed between it and the substrate with the cells. A robotic arm places a 96 well plate into position under the Z-axis cell pipette and the micropipette dispenses the cell(s) into a well of the 96 well plate containing single cell lysis buffer (eg, 0.1% triton). The cell(s) are lysed and a reverse transcriptase is added with poly dT primer, suitable buffers (eg, SmartSeq v4) and the mixture incubated at 42 C to 50 C. A sequencing library is generated by amplification through PCR of heavy and light chain cDNA's with a multiplexed primer library capable of amplifying human TCR genes (and the ScFv fragment present in the CAR if present) and appropriate 5′ and 3′ tags needed for loading of molecules onto an Illumina MiSeq sequencer. Optionally additionally primers are included for amplifying cDNA's from the antigen/target library. Sequences of TCR/CAR fragments are compared with the a. binding/killing/T Cell activation profile b. cellular presence/morphology as determined by the microscope images, and c. sequences from the optional antigen/target library. cDNA's harvested from single cells are used as a template in PCR to amplify and perform molecular cloning of the TCR/CAR fragments into a suitable expression vector. cDNA's from antigen/target cells that are expected to be responsible for T cell activation of the effector library are used as a template in PCR to amplify, or synthesized based on sequencing data, and cloned into a suitable expression vector for further characterization.

### Example 42. Screening of Antibodies Bound to Substrate

Human primary B cells are harvested from a patient exposed to influenza antigen. The cells are treated with appropriate growth factors and cytokines to induce plasmablast/plasma cell differentiation. A substrate is made with a 6 well plastic microplate containing well bottoms of 170 microns thick glass. Each well bottom is patterned with a UV cured PEG hydrogel to yield wells of 100 um×100 um×100 um. The PEG hydrogel is functionalized with carboxyl groups that are attached via EDC/NHS chemistry to protein A/G and/or anti-human Fc antibodies. Plasma cells are loaded onto to the device. A porous membrane is placed approximately 500 microns over the cells to facilitate washing the array. One micron fluorescent beads are placed into the array at a loading rate of approximately 5% of wells to serve as fiducial marks later. The cells/beads/wells are incubated for 1-4 hours and imaged with the automated microscope. The cells/beads/wells are washed with FcBlock (BD Biosciences). The cells/beads/wells are washed with a PBS/BSA mixture and imaged with the automated microscope. The cells/beads/wells are subjected to at least one cycle of (1) incubation with a mixture of fluorescently stained antigen, and (2) imaging with the automated microscope, and (3) washing with PBS/BSA/FcBlock and (4) increasing the antigen concentration and/or changing the antigen. The images from the microscope are processed to identify cell presence and morphology, fluorescence signal of surface bound antigen with respect to cycle number and antigen exposure time, location of fiducials, and the location of cells that have secreted protein signal of the desired binding profile (in this case, binding of at least one strain of influenza antigen with an IgG or IgA antibody and absence of binding to human serum albumin). The perfusion membrane is slowly retracted while adding fresh PBS-BSA medium. A cell picking worklist is generated to select cells fitting the desired binding profile. The microscope images each cell to be selected, ensuring registration of fiducials, and adjusting the expected absolute coordinates of each desired cell as needed. The Z-axis cell pipette aspirates a cell from the target position and retracts so that a receiver plate can be placed between it and the substrate with the cells. A robotic arm places a 96 well plate into position under the Z-axis cell pipette and the cell dispenses the cell into a well of the 96 well plate containing single cell lysis buffer (eg, 0.1% triton). The cell is lysed and a reverse transcriptase is added with poly dT primer, with suitable buffers (eg, SmartSeq v4) and the mixture incubated at 42 C to 50 C. A sequencing library is generated by amplification through PCR of heavy and light chain cDNA's with a multiplexed primer library capable of amplifying human antibody fragments and appropriate 5′ and 3′ tags needed for loading of molecules onto an Illumina MiSeq sequencer. Sequences of antibody fragments are compared to the binding profile and cellular presence/morphology as determined by the microscope. cDNA's harvested from single cells are used as a template in PCR to amplify and perform molecular cloning of the antibody fragments into a suitable IgG expression vector.

### Example 43. Screening of Antibodies Bound to a Substrate by Sequencing of Bound Products

Human primary B cells are harvested from a patient exposed to influenza antigen. The cells are treated with appropriate growth factors and cytokines to induce plasma cell differentiation. A substrate is made with a 6 well plastic microplate containing well bottoms of 170 microns thick glass. Each well bottom is patterned with a UV cured PEG hydrogel to yield wells of 100 um×100 um×100 um. Plasma cells and magnetic beads bound to protein A/G and/or anti-human Fc (polyclonal for IgG1-4,IgA,IgM,IgE,IgD) are loaded onto to the device. A porous membrane is placed approximately 500 microns over the cells to facilitate washing the array. One micron fluorescent beads are placed into the array at a loading rate of approximately 5% of wells to serve as fiducial marks later. The cells/beads/wells are incubated for 1-4 hours and imaged with the automated microscope. The cells/beads/wells are washed with FcBlock (BD Biosciences). The cells/beads/wells are washed with a PBS/BSA mixture and imaged with the automated microscope. The cells/beads/wells are subjected to at least one cycle of (1) incubation with a mixture of fluorescently stained antigen, and (2) imaging with the automated microscope, and (3) washing with PBS/BSA/FcBlock and (4) increasing the antigen concentration and/or changing the antigen. The images from the microscope are processed to identify cell presence and morphology, fluorescence signal of bead bound antigen with respect to cycle number and antigen exposure time, location of fiducials, and the location of cells that have secreted protein signal of the desired binding profile (in this case, binding of at least one strain of influenza antigen with an IgG or IgA antibody and absence of binding to human serum albumin). The perfusion membrane is slowly retracted while adding fresh PBS-BSA medium. A cell picking worklist is generated to select cells fitting the desired binding profile and optionally the beads that are in the vicinity of the cell. The beads are optionally aspirated with the cell or separately. The microscope images each cell/bead to be selected, ensuring registration of the motion control coordinate system to the coordinate system of the image field, and adjusting the motion control coordinates of each desired cell as needed. The Z-axis cell pipette aspirates a cell from the target position and retracts so that a receiver plate can be placed between it and the substrate with the cells. A robotic arm places a 96 well plate into position under the Z-axis cell pipette and the cell dispenses the cell into a well of the 96 well plate containing single cell lysis buffer (eg, 0.1% triton). The cell is lysed and a reverse transcriptase is added with poly dT primer, with suitable buffers (eg, SmartSeq v4) and the mixture incubated at 42 C to 50 C. A sequencing library is generated by amplification through PCR of heavy and light chain cDNA's with a multiplexed primer library capable of amplifying human antibody fragments and appropriate 5′ and 3′ tags needed for loading of molecules onto an Illumina MiSeq sequencer. Nucleic acids present on or in the antigen are optionally amplified as well, and attached to appropriate sequencing tags for loading of molecules onto an Illumina MiSeq sequencer. Sequences of antibody fragments are compared to the binding profile and cellular presence/morphology as determined by the microscope, and sequences of nucleic acids on/in bound antigen in the well of the cell. cDNA's harvested from single cells are used as a template in PCR to amplify and perform molecular cloning of the antibody fragments into a suitable IgG expression vector.

### Example 44: Agglutination Assay. Free Antigen

Human primary B cells are harvested from a patient exposed to influenza antigen. The cells are treated with appropriate growth factors and cytokines to induce plasmablast/plasma cell differentiation. A substrate is made with a 6 well plastic microplate containing well bottoms of 170 microns thick glass. Each well bottom is patterned with a UV cured PEG hydrogel to yield wells of 100 um×100 um×100 um. Plasma cells and 1 micron particles bound to protein A/G and/or anti-human Fc (polyclonal for IgG1-4,IgA,IgM,IgE,IgD) are loaded onto to the device. Polyvalent influenza antigen is introduced at a concentration of approximately 0.1-100 nM, which causes agglutination of beads bound to antibodies. Plasma cells proximal to sites of agglutination and/or attached to beads (as influenza may agglutinate beads to cells as well) are selected with the device and placed into a 96 well plate for subsequent reverse transcription, amplification and sequencing of immune receptors.

### Example 45 Antibodies Against Influenza Virus

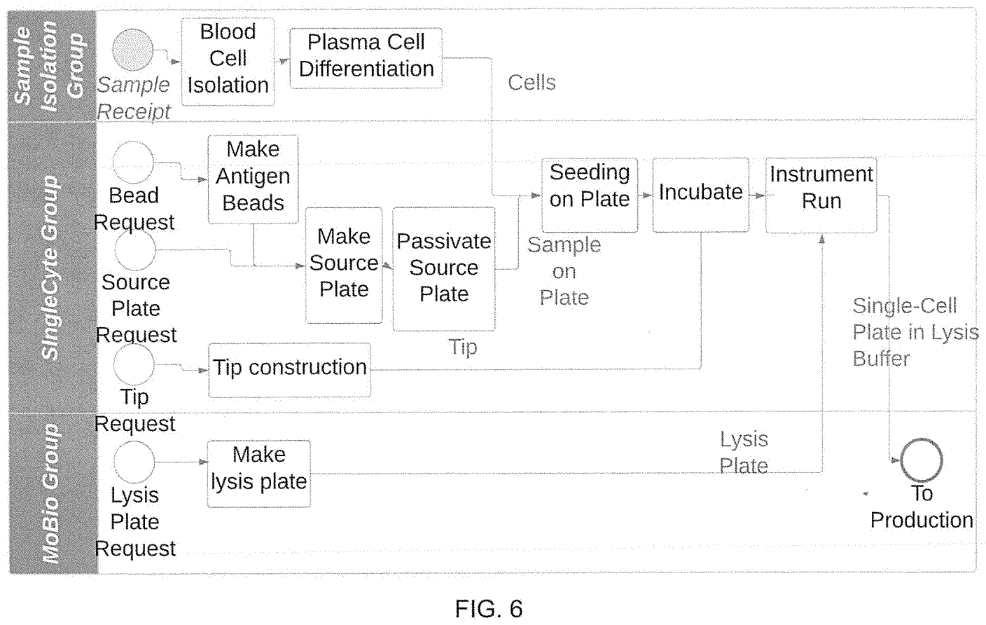

FIG. 6 shows one version of a work flow for making antibodies against influenza virus. In this work flow, blood/serum samples are obtained from patients who have mounted an immune response to influenza (e.g., from a influenza vaccination or from contracting influenza by exposure to other infected patients). The blood/serum was depleted of T-cells and these T-cell depleted PBMC's were loaded onto a substrate. A solution of Pierce 1 micron beads bound to influenza antigens (e.g., hemagglutinin, neuraminidase, NB protein, Matrix protein 1 or 2) were added to the substrate with a goat anti-human Fab antibody bound to R-phycoerythrin (“secondary antibody”). The cells and beads and secondary antibody were incubated for 24 hours under appropriate culture conditions. The microscope is used to identify halos of fluorescent beads that have captured cells that secreted antibodies and were stained with the secondary antibody. Cells with halos were selected with the device and placed into 96 well plates where subsequent molecular biology results in amplification and sequencing of immune receptor nucleic acids.

After picking single cells into lysis buffer, cells were lysed at 70C in the presence of poly dT primer. RT buffer and reverse transcriptase were added and single cell cDNA produced at 55C. Gene specific amplification of heavy and light chains was performed after the addition of DNA polymerase (Kapa HiFi), buffer and primers and PCR thermal cycling. Amplified genes were barcoded with well specific barcodes in a subsequent PCR reaction, all cDNA's for a single chain from a plate pooled, chain/plate libraries barcoded with chain/plate specific barcodes in a subsequent PCR reaction, and then chain/plate libraries normalized and pooled before loading on an Illumina MiSeq sequencer. Reads were separated by their plate/well/chain and put through an analysis pipeline that involved clustering reads based on sequence entropy to make a consensus assembly, consensus sequences found by aligning all reads in a well/chain/plate barcode group to each assembly and making basecalls by consensus, and then annotating each sequence by alignment with IgBlast against a human germline reference database. Paired antibody genes were then amplified via PCR with cloning tags or synthesized, cloned into an expression vector and expressed in HEK293 cells. Full length antibodies were assayed for binding with ForteBio and Luminex assays.

Table 1 below shows representative examples of antibody clones obtained against influenza virus antigens.

TABLE 1
Exemplary Antibody Clones

| Clone Name | Isotype | Antigen | Sequence | CDR3 |
|---|---|---|---|---|
| BNB-A01- | IGHA2*03 | Influenza | SEQ ID NO: 1 | SEQ ID NO: 11 |
| Heavy | | H3N2 | | |
| BNB-A01- | IGHA2*03 | Influenza | SEQ ID NO: 2 | SEQ ID NO: 12 |
| Heavy | | H3N2 | | |
| BNB-A01- | IGLC7*03 | Influenza | SEQ ID NO: 3 | SEQ ID NO: 13 |
| Light | | H3N2 | | |
| BNB-A01- | IGLC7*03 | Influenza | SEQ ID NO: 4 | SEQ ID NO: 14 |
| Light | | H3N2 | | |
| BNB-A02- | IGHA2*03 | Influenza | SEQ ID NO: 5 | SEQ ID NO: 15 |
| Heavy | | H3N2 | | |
| BNB-A02- | IGHG3*04 | Influenza | SEQ ID NO: 6 | SEQ ID NO: 16 |
| Heavy | | H3N2 | | |
| BNB-A02- | IGK | Influenza | SEQ ID NO: 7 | SEQ ID NO: 17 |
| Light | | H3N2 | | |
| BNB-A02- | IGKC*05 | Influenza | SEQ ID NO: 8 | SEQ ID NO: 18 |
| Light | | H3N2 | | |
| BNB-A03- | IGHG3*04 | Influenza | SEQ ID NO: 9 | SEQ ID NO: 19 |
| Heavy | | H3N2 | | |
| BNB-A03- | IGHA2*03 | Influenza | SEQ ID NO: 10 | SEQ ID NO: 20 |
| Heavy | | H3N2 | | |

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

Those skilled in the art will recognize or be able to ascertain using no more than routine experimentation, many equivalents to the specific embodiments of the invention described herein. Such equivalents are intended to be encompassed by the following claims.

We claim:

1. A method for obtaining the sequence of a secreted protein, comprising the steps of:
a. obtaining a first plurality of cells that secrete a binding protein that can bind an antigen;
b. derivatizing a substrate with a plurality of carriers bound to the antigen;
c. adding to the substrate the first plurality of cells that secrete the binding proteins;
c. providing a high-throughput screening device comprising an inverted microscope and a camera component, the substrate, a cell picker component; and a robotic arm component, wherein the high-throughput screening device is capable of isolating a cell from a heterogeneous population of cells;
d. interrogating the first plurality of cells for the secreted binding protein that binds the antigen on the plurality of carriers for an optical signal that is used as a screening criteria, wherein the optical signal is associate with the plurality of carriers bound to the antigen;
e. using the high-throughput screening device to pick a second plurality of cells that are positive for the optical signal, and placing the second plurality of cells onto individual positions in a receiver plate;
f. amplifying nucleic acids from the second plurality of cells; and
g. sequencing the nucleic acids.

2. The method of claim 1, wherein the first plurality of cells are lymphocytes.

3. The method of claim 1, wherein the first plurality of cells are lymphocytes from a patient who has mounted an immune response.

4. The method of claim 1, wherein the carrier of the antigen is a bead.

5. The method of claim 1 wherein the carrier of the antigen is a cell.

6. The method of claim 1, wherein the antigen is from an infectious agent or a virus.

7. The method of claim 1, wherein the optical signal is a change in the spatial distribution of the plurality of antigen carriers.

8. The method of claim 1, wherein the carrier of the antigen is a cell, and binding of the cell activates a receptor which elicits the response of a fluorescent reporter, which is the optical signal.

9. The method of claim 1, wherein a fluorescent secondary antibody binds the binding protein secreted from the first plurality of cells, wherein the fluorescent antibody emits the optical signal under stimulation.

10. The method of claim 1, wherein a third molecule interferes with binding of the secreted binding proteins by the antigen, and the selection criteria is a decrease in signal intensity.

11. The method of claim 1, wherein the secreted binding protein is an antibody.

12. The method of claim 1, wherein the secreted binding protein is a soluble TCR.

13. The method of claim 1, wherein the secreted binding protein is a soluble MHC domain.

14. The method of claim 1, wherein the antigen is an MHC protein.

15. The method of claim 1, wherein the antigen is a TCR.

16. The method of claim 1, wherein the antigen is a chimeric antigen receptor.

17. The method of claim 1, wherein the carrier is a cell and binding of the binding protein elicits a change in morphology of the carrier, which is read optically.

18. The method of claim 1, wherein the antigen is selected from the group consisting of a hemagglutinin, a NB protein, a neuraminidase, a SARS-CoV spike protein, a coronavirus, a herpes virus, HSV gD protein, HSV gG protein, and an influenza virus.

19. The method of claim 1, wherein the binding protein neutralizes a virus as demonstrated by the ability of the binding protein to protect susceptible cells in vitro from infection by the virus.

20. The method of claim 1, wherein the interrogating step includes a plurality of different antigens, wherein each different antigen carrier is labeled with a different label.

21. The method of claim 20, wherein the carrier of the antigen has a nucleic acid with a particular sequence that can be used for identification.

22. The method of claim 20, wherein each carrier of the antigen has a fluorophore that can be used for identification of a subset of the plurality of carriers of the antigen.

23. The method of claim 20, wherein each carrier of the antigen has a physical geometry that can be used for identification of a subset of the plurality of carriers of the antigen.

24. The method of claim 20, wherein the carrier of the antigen is a cancer cell.

25. The method of claim 1, wherein the carrier has antibodies that bind the secreted binding protein.


SEQUENCE LISTING (20 sequences)

SEQ ID NO: 1 - DNA, 468 bp, Homo sapiens
gaagaccttggggctggtcggggatgctgaggagacggtgactgtggtccctttgccccagacgtctatgtagtagtcctggtcgagaccaacgttaagaccagggaccagctctctggcacagtaataaacggccgtgtccgcggcggtcacagaactcaacttcagggagaattggttgttggacgtgtcaactgacatgatgactcgactcttgagtaaggggttgtagtgagtgatcccattatgggagacgtacccgatccactccaggcccttcccgggcggctggcggatccaactccaggagtaatcaccactactgactgagccaccaaagacagagcaggtgagggacagggtctgtgcaggcttcacgagtcctgagccggactcctgcagctgcatctgggactggacccatctgggagctgccaccagcaggaggaagaaccacaggtgtttcat

SEQ ID NO: 2 - DNA, 441 bp, Homo sapiens
gaagaccttggggctggtcggggatgctgaggagaccctgaccgttgtccctgtgccccagacgtccattcgaaagctgccacgcggatctctcgcacagtaataagtagccgtatcgtcgactgtcaggctgttcatttggagatgcaccgacatgtcggcgttgtctctggacatggtgaatcggcccctcattgactcggcatagaaaatgaaagaactatcagtattaatggaagcgacccactccaggcccttccctggagcctggcggacccagttgatggtgtattcagcaaagatgaacccagagcctttacacgagagtctcagagaccccccaggcgagaccaggcctcccccagattccaccagtttcacgtcacactggacaccttctaaaatagcaacaaggaaaacccagcggagccccagttccat

SEQ ID NO: 3 - DNA, 394 bp, Homo sapiens
tcctcagaggagggtgggaacagagtgaccgagggggcagccttgggctgacctaggacggtcagcttggtccctccgccgaacacccaagcagtcaggctggcatcccatactgcacagaaataatcagcctcatcctcgggccggagcccactgatggccagggaggctgaggcgccagacgtggagccagagaatcggtcagggacccctgagggccgcttgtcatggctatatatgaggagtttgggggccgctcctgggaattgctggaaccagcacacataattagttccgatgttggagctggttccagaacaagagatggtgaccctctgcccgggggtcccagacgctgagggtggctgagtcagtacagactgggcccaggacc

SEQ ID NO: 4 - DNA, 394 bp, Homo sapiens
tcctcagaggagggtgggaacagagtgaccgagggggcagccttgggctgacttaggacggtcactttggtccctccgccgaaaaccaaagtactcaggacgtcatcccatgttgcgcagaaataatcagcctcatcctcaggctggagcgcactgatggccagggaggctgaggtgccagacttggagccagagaagcggtcagagacccccgaggggcgctgatcattagtgtaaatgaggagtttgggggccgcccctgggacttgttgataccaggccacaggattactcccgatattggagttggtccccgaacaagacatggtgaccctctgtccgggggtcccagaggctgagggtggctgagtcagcacagactgggcccaggacc

SEQ ID NO: 5 - DNA, 441 bp, Homo sapiens; identical to SEQ ID NO: 2

SEQ ID NO: 6 - DNA, 452 bp, Homo sapiens
aagaccgatgggcccttggtggaggctgaggagacggtgaccgtggtcccctggccccagacgtccatactccaaacatgtcgtactcccacaacatctcctctcgcacaataatagacagccgtgtcctcagttctcagggtgttcatgtccaaatccagggtgctcttggaattgtctctggagatggcgaatcggcccttcacggagtcaacgtacgaatatttagttccatcaaatgatatagctgccacccactctggccccttgcctggaggctggcggacccagtgcatagcataattggcgaaggtgaatccagaggctgcacaggagagtctcagggacctcccaggctggaccacgcctcccccagactccaccacctgcacctgacactggacacctcttaaaagagcaacgaggaaaacccagctcagcccaaactccat

SEQ ID NO: 7 - DNA, 376 bp, Homo sapiens
agatggtgcagccacagttcgtttgatctccagcttggtcccctggccaaaagtgcacgagtaatcatactgctgacagtaatacactgcaaaatcttcaggctccagtctgctgatggtgagagtgaagtctgtcccggtcccactgccactgaacctgtctgggatgccagtggccctgctggatgcaccatagatgaggagcctgggagcctggccaggtttttgctggtaccaggctaagcggctgctgctaacactctgactggccctgcaggagagggtggctctttcccctggagacaaagacagggtgcctggagactgcgtcaacacaatttctccggtggtatctgggagccagagtagcaggagg

SEQ ID NO: 8 - DNA, 376 bp, Homo sapiens
agatggtgcagccacagttcgattgatctccaccctggtccctccgccgaacgtgggtgaactaccatgctgatgacagtaaaagactgcaaaatcttcaggctcaagtctgctgattgtgagagtgtagtctgtcccagacccactgccactgaacctgtctgggatgtcagtggccctattagatgcaccatagatgaggagcctgggagcctggccaggtttctggatgtaccaggcgaagtaggtgctgctaacagggtgactggccctgcaggagagggtggcgctttcccctggagacaaagatagggtgcctggagactgcgtcaacagaatttgtccggcggaatctgggagccagagtagcaggagg

SEQ ID NO: 9 - DNA, 452 bp, Homo sapiens; identical to SEQ ID NO: 6

SEQ ID NO: 10 - DNA, 441 bp, Homo sapiens; identical to SEQ ID NO: 2

SEQ ID NO: 11 - DNA, 60 bp, Homo sapiens
gccagagagctggtccctggtcttaacgttggtctcgaccaggactactacatagacgtc

SEQ ID NO: 12 - DNA, 36 bp, Homo sapiens
gcgagagatccgcgtggcagctttcgaatggacgtc

SEQ ID NO: 13 - DNA, 33 bp, Homo sapiens
gcagtatgggatgccagcctgactgcttgggtg

SEQ ID NO: 14 - DNA, 33 bp, Homo sapiens
gcaacatgggatgacgtcctgagtactttggtt

SEQ ID NO: 15 - DNA, 36 bp, Homo sapiens; identical to SEQ ID NO: 12

SEQ ID NO: 16 - DNA, 48 bp, Homo sapiens
gcgagaggagatgttgtgggagtacgacatgtttggagtatggacgtc

SEQ ID NO: 17 - DNA, 24 bp, Homo sapiens
cagcagtatgattactcgtgcact

SEQ ID NO: 18 - DNA, 24 bp, Homo sapiens
catcagcatggtagttcacccacg

SEQ ID NO: 19 - DNA, 48 bp, Homo sapiens; identical to SEQ ID NO: 16

SEQ ID NO: 20 - DNA, 36 bp, Homo sapiens; identical to SEQ ID NO: 12